(19) United States
(12) Patent Application Publication
(10) Pub. No.: US 2014/0030722 A1
(43) Pub. Date: Jan. 30, 2014

(54) NLRP7-BASED DIAGNOSIS OF FEMALE REPRODUCTIVE CONDITIONS

(71) Applicant: Rima Slim, Montreal (CA)

(72) Inventor: Rima Slim, Montreal (CA)

(73) Assignee: THE ROYAL INSTITUTION FOR THE ADVANCEMENT OF LEARNING/MCGILL UNIVERSITY, MONTREAL, QC (CA)

(21) Appl. No.: 14/005,093

(22) PCT Filed: Mar. 16, 2012
(86) PCT No.: PCT/CA2012/050165

§ 371 Date: Oct. 15, 2013

Related U.S. Application Data
(60) Provisional application No. 61/453,720, filed on Mar. 17, 2011.

(51) Int. Cl. C12Q 1/68; G01N 33/68

(52) U.S. Cl. CPC C12Q 1/6883; G01N 33/6893
USPC 435/6.12; 536/24.33; 530/387.9; 435/7.92

(57) ABSTRACT

Methods, reagents and kits are described for the diagnosis of a female reproductive condition such as reproductive wastage, based on the detection of an alteration in a NLRP7-encoding nucleic acid or a NLRP7 polypeptide, relative to a corresponding wild-type NLRP7-encoding nucleic acid or NLRP7 polypeptide.

### CROSS REFERENCE TO RELATED APPLICATIONS

This application claims the benefit of U.S. Provisional Patent Application Ser. No. 61/453,720 filed on Mar. 17, 2011, which is incorporated herein by reference in its entirety.

### FIELD OF THE INVENTION

The present invention relates to the field of reproductive conditions, and more particularly to methods and reagents for the diagnosis of predisposition to conditions of the female reproductive system such as reproductive wastage.

### BACKGROUND OF THE INVENTION

A number of female reproductive conditions exist which prevent couples from fulfilling their desire of having their own children. The most common of these conditions is recurrent spontaneous abortions (SAs). A spontaneous abortion is defined as the in-utero death of the baby before 20 weeks of gestation. The common form of non-recurrent spontaneous abortions affect a large number of pregnancies (about 30%) and have complex etiologies involving mostly environmental factors. However, recurrent spontaneous abortions, defined by the occurrence of at least 3 spontaneous abortions (3 SAs), affect about 5% of couples who are trying to conceive. Recurrent spontaneous abortions have a complex etiology, involving strong genetic predisposition because of their recurrence of three times or more. Other related forms of early and late fetal losses include blighted ovum, ectopic pregnancy, and stillbirth, all of which are found in women with molar pregnancies or hydatidiform moles. A molar pregnancy or a hydatidifrom mole (HM) is an aberrant human pregnancy characterized by abnormal embryonic development, hydropic degeneration of chorionic villi, and proliferation of the trophoblast. Common moles are sporadic, usually not recurrent, and have a complex etiology involving both genetic and environmental factors. Common moles occur once in every 600 pregnancies in western societies [1]. Among women with one mole, 1-6% will develop a second mole [2, 3, 4, 5, 6, 7], depending on populations and studies, and 10-25% will experience a second reproductive wastage (RW), mostly as a spontaneous abortion (SA) [2, 4, 8, 9]. All these forms of early and late fetal losses are inter-related and in fact alternate in the same patients demonstrating their common underlying causes, at least in some cases. These various forms of fetal losses are referred to hereafter as recurrent reproductive wastage.

Recurrent HM is a rare clinical entity in which molar tissues are diploids and have usually a biparental contribution to their genome. In a number of cases this condition has been observed to have a familial basis. Recurrent hydatidiform molar tissues with diploid biparental contribution, in general, have less trophoblast proliferation than androgenetic moles.

Molar pregnancies are first diagnosed based on ultrasonography and high serum levels of beta-hCG. Definitive diagnosis is made after histopathological examination of the evacuated products of conception (POCs), which allows dividing them into complete and partial moles and distinguishing them from nonmolar spontaneous abortions. At the histopathological level, complete hydatidiform moles (CHMs) do not contain embryonic tissues other than the chorionic villi and have excessive trophoblast proliferation [10]. Partial moles (PHMs) may contain other embryonic tissues (amnion, chorion, or others) but have mild and focal trophoblastic proliferation [10]. Nonmolar spontaneous abortions may contain embryonic tissues but most do not have trophoblastic proliferation. Because histopathology is a descriptive, qualitative science and lacks quantitative measurements to assess the degree and extent of trophoblastic proliferation (mild, excessive, focal, occasional, etc.), there is a wide interobserver and intraobserver variability mainly in distinguishing PHM from SA and in distinguishing CHM from PHM [11]. In addition, epidemiological studies have shown that the frequency of moles is higher in patients with recurrent spontaneous abortions than in women from the general population [12, 13]. Also, a history of recurrent spontaneous abortions is a known risk factor for moles [14]. Furthermore, women with recurrent moles may have CHM, PHM, and SAs indicating that these three histopathological entities have, at least in some cases, the same underlying etiology and are rather a continuous spectrum of the same condition.

Therefore, there is a continued need to identify gene alterations and markers associated with recurrent reproductive wastage.

The present description refers to a number of documents, the content of which is herein incorporated by reference in their entirety.

### SUMMARY OF THE INVENTION

The invention relates to NLRP7 and its association with recurrent forms of reproductive wastage, including diagnosis of such conditions based on the detection of alterations in NLRP7.

The invention provides a method for diagnosing a predisposition for recurrent reproductive wastage (e.g., various forms of recurrent fetal losses) in a female (e.g., human) subject, the method comprising detecting an alteration in the sequence of a NLRP7 nucleic acid or encoded polypeptide in a sample from said subject relative to a wild-type (native) NLRP7 nucleic acid or encoded polypeptide sequence, wherein the presence of said alteration indicates that the subject has a predisposition for a recurrent reproductive wastage condition.

In an aspect, the present invention provides a method for diagnosing a predisposition for recurrent reproductive wastage in a female subject, the method comprising detecting one or more alterations in the sequence of a NLRP7 nucleic acid or encoded polypeptide in a sample from said subject, relative to the sequence of a wild-type NLRP7 nucleic acid or encoded polypeptide, wherein said one or more alterations are nonsynonymous mutations causing an amino acid change at one or more positions corresponding to residues 250, 310, 311, 319, 340, 390, 413, 427, 430, 481, 487, 511, 659, 851, 872, and 931 in the NLRP7 polypeptide sequence of SEQ ID NO: 7, wherein the presence of said one or more alterations is indicative that the female subject has a predisposition for recurrent reproductive wastage.

In an embodiment, the above-mentioned nonsynonymous mutation is a nucleotide substitution.

In an embodiment, the above-mentioned nonsynonymous mutation is a nucleotide deletion.

In an embodiment, the above-mentioned nonsynonymous mutation causes a Phe to Leu change at a position corresponding to residue 250 in the NLRP7 polypeptide sequence of SEQ ID NO: 7. In a further embodiment, the above-mentioned nucleotide substitution is a C to A substitution at a position corresponding to nucleotide 750 in the NLRP7 nucleotide sequence of SEQ ID NO:8.

In an embodiment, the above-mentioned nonsynonymous mutation causes a Gln to His change at a position corresponding to residue 310 in the NLRP7 polypeptide sequence of SEQ ID NO: 7. In a further embodiment, the above-mentioned nucleotide deletion is a deletion of a GC dinucleotide at positions corresponding to nucleotides 930 and 931 in the NLRP7 nucleotide sequence of SEQ ID NO:8.

In an embodiment, the above-mentioned nonsynonymous mutation causes a Glu to Gln change at a position corresponding to residue 340 in the NLRP7 polypeptide sequence of SEQ ID NO: 7. In a further embodiment, the above-mentioned nucleotide substitution is a GAG to CAAAA substitution at positions corresponding to nucleotides 1018 to 1020 in the NLRP7 nucleotide sequence of SEQ ID NO:8.

In an embodiment, the above-mentioned nonsynonymous mutation causes a Glu to Lys change at a position corresponding to residue 340 in the NLRP7 polypeptide sequence of SEQ ID NO: 7. In a further embodiment, the above-mentioned nucleotide substitution is a G to A substitution at positions corresponding to nucleotide 1018 in the NLRP7 nucleotide sequence of SEQ ID NO:8.

In an embodiment, the above-mentioned nonsynonymous mutation causes an Arg to His change at a position corresponding to residue 390 in the NLRP7 polypeptide sequence of SEQ ID NO: 7. In a further embodiment, the above-mentioned nucleotide substitution is a G to A substitution at positions corresponding to nucleotide 1169 in the NLRP7 nucleotide sequence of SEQ ID NO:8.

In an embodiment, the above-mentioned nonsynonymous mutation causes an Arg to Trp change at a position corresponding to residue 413 in the NLRP7 polypeptide sequence of SEQ ID NO: 7. In a further embodiment, the above-mentioned nucleotide substitution is a C to T substitution at positions corresponding to nucleotide 1237 in the NLRP7 nucleotide sequence of SEQ ID NO:8.

In an embodiment, the above-mentioned nonsynonymous mutation causes an Arg to Leu change at a position corresponding to residue 659 in the NLRP7 polypeptide sequence of SEQ ID NO: 7. In a further embodiment, the above-mentioned nucleotide substitution is a G to T substitution at positions corresponding to nucleotide 1976 in the NLRP7 nucleotide sequence of SEQ ID NO:8.

In an embodiment, the above-mentioned nonsynonymous mutation causes an Arg to His change at a position corresponding to residue 815 in the NLRP7 polypeptide sequence of SEQ ID NO: 7. In a further embodiment, the above-mentioned nucleotide substitution is a G to A substitution at positions corresponding to nucleotide 2444 in the NLRP7 nucleotide sequence of SEQ ID NO:8.

In an embodiment, the above-mentioned nonsynonymous mutation causes a Tyr to Stop change at a position corresponding to residue 872 in the NLRP7 polypeptide sequence of SEQ ID NO: 7. In a further embodiment, the above-mentioned nucleotide substitution is a C to A substitution at positions corresponding to nucleotide 2616 in the NLRP7 nucleotide sequence of SEQ ID NO:8.

In an embodiment, the above-mentioned nonsynonymous mutation causes a premature termination of the NLRP7 polypeptide at a position corresponding to residue 931 in the NLRP7 polypeptide sequence of SEQ ID NO: 7. In a further embodiment, the above-mentioned nucleotide deletion is a TG deletion at positions corresponding to nucleotides 2791 and 2792 in the NLRP7 nucleotide sequence of SEQ ID NO:8.

In an embodiment, the above-mentioned nonsynonymous mutation causes a Gln to Arg change at a position corresponding to residue 310 in the NLRP7 polypeptide sequence of SEQ ID NO: 7. In a further embodiment, the above-mentioned nucleotide substitution is an A to G substitution at a position corresponding to nucleotide 929 in the NLRP7 nucleotide sequence of SEQ ID NO:8.

In an embodiment, the above-mentioned nonsynonymous mutation causes a Leu to Ile change at a position corresponding to residue 311 in the NLRP7 polypeptide sequence of SEQ ID NO: 7. In a further embodiment, the above-mentioned nucleotide substitution is a C to A substitution at a position corresponding to nucleotide 931 in the NLRP7 nucleotide sequence of SEQ ID NO:8.

In an embodiment, the above-mentioned nonsynonymous mutation causes a Val to Ile change at a position corresponding to residue 319 in the NLRP7 polypeptide sequence of SEQ ID NO: 7. In a further embodiment, the above-mentioned nucleotide substitution is a G to A substitution at a position corresponding to nucleotide 955 in the NLRP7 nucleotide sequence of SEQ ID NO:8.

In an embodiment, the above-mentioned nonsynonymous mutation causes a Met to Thr change at a position corresponding to residue 427 in the NLRP7 polypeptide sequence of SEQ ID NO: 7. In a further embodiment, the above-mentioned nucleotide substitution is a T to C substitution at a position corresponding to nucleotide 1280 in the NLRP7 nucleotide sequence of SEQ ID NO:8.

In an embodiment, the above-mentioned nonsynonymous mutation causes an Ala to Thr change at a position corresponding to residue 481 in the NLRP7 polypeptide sequence of SEQ ID NO: 7. In a further embodiment, the above-mentioned nucleotide substitution is a G to A substitution at a position corresponding to nucleotide 1441 in the NLRP7 nucleotide sequence of SEQ ID NO:8

In an embodiment, the above-mentioned nonsynonymous mutation causes a Gly to Glu change at a position corresponding to residue 487 in the NLRP7 polypeptide sequence of SEQ ID NO: 7. In a further embodiment, the above-mentioned nucleotide substitution is a T to C substitution at a position corresponding to nucleotide 1460 in the NLRP7 nucleotide sequence of SEQ ID NO:8.

In an embodiment, the above-mentioned nonsynonymous mutation causes a Lys to Arg change at a position corresponding to residue 511 in the NLRP7 polypeptide sequence of SEQ ID NO: 7.

In a further embodiment, the above-mentioned nucleotide substitution is an A to G substitution at a position corresponding to nucleotide 1532 in the NLRP7 nucleotide sequence of SEQ ID NO:8.

In an embodiment, the female subject is undergoing, or is a candidate for, assisted reproductive technologies (ART).

In another aspect, the present invention provides a method for determining whether an embryo or oocyte carries a genetic predisposition for a reproductive condition, the method comprising detecting one or more of the alterations in the sequence of a NLRP7 nucleic acid or encoded polypeptide defined above in said embryo or oocyte, wherein the presence of said alteration is indicative that said embryo or oocyte carries a genetic predisposition for a reproductive condition.

In another aspect, the present invention provides an oligonucleotide capable of specifically hybridizing, under stringent conditions, to the above-mentioned altered NLRP7 nucleotide sequence and not to a corresponding wild-type NLRP7 nucleotide sequence.

In another aspect, the present invention provides an isolated altered NLRP7 polypeptide comprising one or more of the amino acid changes defined above.

In another aspect, the present invention provides an isolated NLRP7 nucleic acid encoding the above-mentioned altered NLRP7 polypeptide.

In another aspect, the present invention provides a vector comprising the above-mentioned isolated NLRP7 nucleic acid.

In another aspect, the present invention provides a host cell transformed with the above-mentioned vector.

In another aspect, the present invention provides an antibody capable of specifically binding to the above-mentioned altered NLRP7 polypeptide.

In another aspect, the present invention provides a kit for diagnosing a predisposition for recurrent reproductive wastage in a female subject, said kit comprising a reagent for detecting an alteration in the sequence of a NLRP7 nucleic acid or encoded polypeptide in a sample from said subject, relative to the sequence of a wild-type NLRP7 nucleic acid or encoded polypeptide, wherein said one or more alterations are nonsynonymous mutations causing an amino acid changes at one or more positions corresponding to residues 250, 310, 311, 319, 340, 390, 413, 427, 430, 481, 487, 511, 659, 851, 872 and 931 in the NLRP7 polypeptide sequence of SEQ ID NO: 7.

In an embodiment, the above-mentioned reagent for detecting is the above-mentioned oligonucleotide or antibody.

In an embodiment, the above-mentioned reproductive condition is reproductive wastage, in a further embodiment a condition selected from hydatidiform mole, spontaneous abortion, blighted ovum, elective termination, stillbirth, placental abruption, ectopic pregnancy, choriocarcinoma or gestational trophoblastic neoplasia.

In an embodiment, the above-mentioned the alteration is present in one allele of the NLRP7 nucleic acid and said subject is heterozygous for said alteration.

In another embodiment, the above-mentioned alteration is present in both alleles of the NLRP7 nucleic acid and said subject is homozygous for said alteration.

Other advantages and features of the present invention will become more apparent upon reading of the following non-restrictive description of specific embodiments thereof, given by way of example only with reference to the accompanying drawings.

### BRIEF DESCRIPTION OF THE DRAWINGS

FIG. 1: Summary of NLRP7 mutation analysis of 135 unrelated patients with a spectrum of reproductive wastage. NSV stands for non-synonymous variant, SA, for spontaneous abortion, HM, for hydatidiform mole.

FIG. 2: (A) Comparison between the reproductive outcomes of patients with one or two defective alleles. n, indicates the total number of pregnancies of patients from each category. In this figure, only major categories of reproductive wastage are shown, the other rare forms (blighted ovum, ectopic pregnancy, elective abortion, malformed baby) observed in small numbers (between 2 and 6) were either removed or fused with related categories. (B) Correlation between the nature of NLRP7 mutations and the histopathological diagnosis of products of conception (POCs) established by two pathologists. (C) Genomic and protein structures of NLRP7. (D) Distribution of the different types of NLRP7 mutations in its three domains. Distribution of mutations and variants found in the three categories of patients and of mutations listed on INFEVERS (INFEVERS: an online database for autoinflammatory mutations. Copyright. Available at http://fmf.igh.cnrs.fr/ISSAID/infevers/; refs [26 to 28]). “inter”, indicates amino acid between two domains; and “after”, indicates amino acids after the LRR domain. First bar (black)=total mutations or variants; second bar (dark gray)=protein-truncating mutations; third bar (light gray)=missense mutations; fourth bar (white)=non-synonymous variants;

FIG. 3: (A) IL1B and TNF secretion by ex vivo stimulated PBMCs with lipopolysaccharides (LPS) from 5 patients with A481T and other rare NSVs in NLRP7. The patients included in this analysis are 698, 754, 819, 821 and 830. Each patient has one copy of A481T with or without other rare NSVs (Table 5). PBMCs from 7 subjects without A481T and any of the other rare NSVs were used as controls. Patients carrying A481T and other rare NSVs secrete significantly lower amounts of IL1B and TNF than controls not carrying these variants (p<0.0001). (B) Cell lysates of LPS stimulated PBMCs from five patients and the same control were subjected to immunoblots to determine the levels of pro-IL1B and mature IL1B. The levels of mature IL1B were normalized to those of β-actin. IL1B is not constitutively expressed by PBMCs. Upon stimulation, PBMCs from the patients and control produce variable amounts of intracellular pro and mature IL1B as reported in healthy subjects and none of them has defective IL1B processing (C) Comparison of the ratios of patient-to-control (patient/control) of intracellular mature MB, quantitated by the Image J software, and the ratios of secreted IL1B in the extracellular milieu measured by ELISA (patient/control). This analysis showed that patients' cells secrete lower amounts of the produced mature intracellular MB than controls.

FIGS. 4A-4H: Genomic DNA sequence of human NLRP7 (SEQ ID NO: 1; derived from GenBank accession No. NC_{—}000019.8)

FIGS. 5A-5C: Nucleic acid (SEQ ID NO: 2, FIGS. 5A and 5B) and polypeptide (SEQ ID NO: 3, FIG. 5C) sequence of human NLRP7, 980 amino acid isoform (GenBank accession No. NM_{—}206828, isoform 2). The coding sequence is defined by positions 77-3019 of DNA sequence.

FIGS. 6A-6C: Nucleic acid (SEQ ID NO: 4, FIGS. 6A and 6B) and polypeptide (SEQ ID NO: 5, FIG. 6C) sequence of human NLRP7, 1009 amino acid isoform (GenBank accession No. NM_{—}139176, isoform 1). The coding sequence is defined by positions 77-3106 of DNA sequence.

FIGS. 7A-7D: Nucleic acid (SEQ ID NO: 6, FIGS. 7A and 7B) and polypeptide (SEQ ID NO: 7, FIG. 7C) sequence of human NLRP7, 1037 amino acid isoform (GenBank accession No. NM_{—}001127255.1). FIG. 7D depicts the coding (cDNA) sequence of this isoform, which correspond to positions 77-3190 of FIGS. 7A and 7B (SEQ ID NO: 8).

FIG. 8: (A) Histopathology of the placenta from the malformed baby of patient 428 with showing marked chorioamnionitis. (B) Placenta from term pregnancy of patient 754 displaying oedematous and dysmature stem villi with chorangiosis.

FIGS. 9A and 9B: NLRP7 mutations/alterations known to be linked to a predisposition to recurrent reproductive wastage (INFEVERS: an online database for autoinflammatory mutations. Copyright. Available at http://fmf.igh.cnrs.fr/ISSAID/infevers/; refs [26 to 28]). The sequence is depicted in SEQ ID NO: 9.

### DESCRIPTION OF ILLUSTRATIVE EMBODIMENTS

Terms and symbols of genetics, molecular biology, biochemistry and nucleic acid used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like. All terms are to be understood with their typical meanings established in the relevant art.

The articles “a” and “an” are used herein to refer to one or to more than one (i.e. to at least one) of the grammatical object of the article. By way of example, “an element” means one element or more than one element. Throughout this specification, unless the context requires otherwise, the words “comprise,” “comprises” and “comprising” will be understood to imply the inclusion of a stated step or element or group of steps or elements but not the exclusion of any other step or element or group of steps or elements.

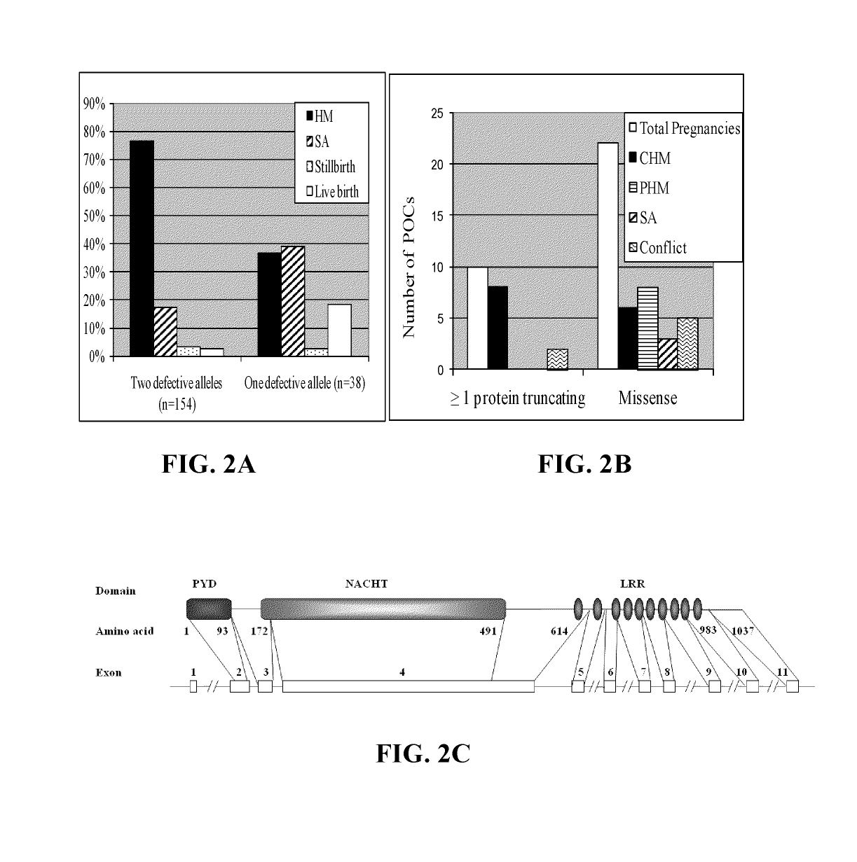

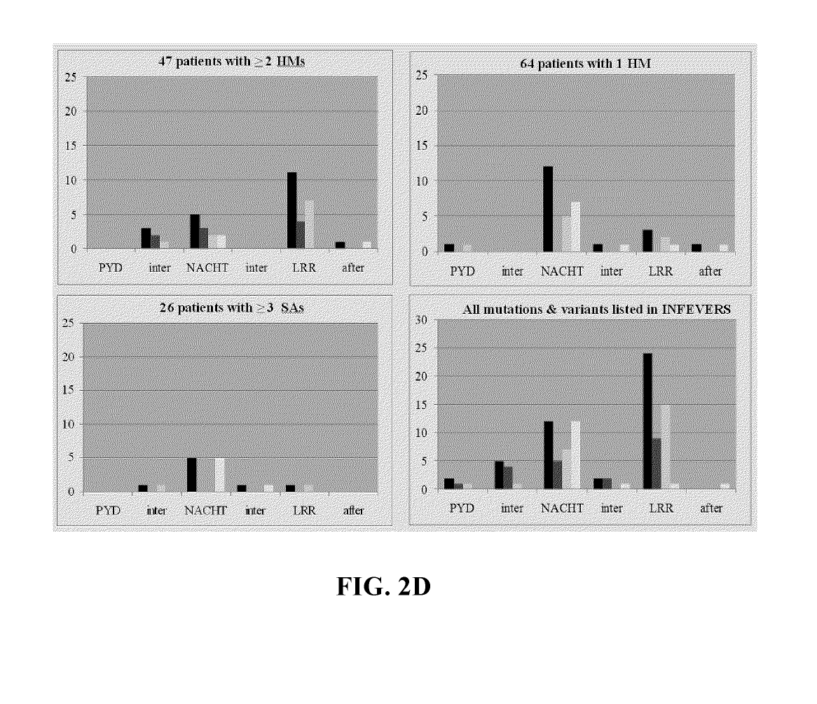

NLRP7 is one of 14 members of the NLRP (also referred to as NALP) proteins, a large subfamily of the CATERPILLER protein family involved in inflammation and apoptosis. NLRP7 is related to the mouse MATER, (also a member of the CATERPILLER protein family). The NLRP7 gene consists of 11 exons encoding for 1037 amino acid protein (the longest isoform, NM_{—}001127255.1). Three transcriptional isoforms NLRP7V1-V3 involving the alternative splicing of exons 5, 9, and 10 have been described (Okada et al., 2004). NLRP7 (NOD-like receptor proteins, pyrin containing domain 7) is formed by three main domains, a pyrin (or DAPIN) domain (corresponding to residues 1-93 in human NLRP7), a NACHT domain (corresponding to about residues 172-491 in human NLRP7), and 9 to 10 leucine rich repeats (LRR) depending on splice isoforms at the C-terminus (FIG. 2C). NLRP7 has been shown to play a causal role in recurrent HMs (RHMs) and associated reproductive wastage [15]. Based on the original gene identification, then mutations in this gene have been reported by various groups and in patients from several populations demonstrating that NLRP7 is a gene involved in recurrent reproductive wastage [16, 17, 18, 19, 20, 21, 22] (http://fmf.igh.cnrs.fr/ISSAID/infevers/). In vitro, NLRP7 overexpression inhibits caspase-1 dependent interleukin 1 beta (IL1B) secretion [24].

Because of the diagnostic overlap between moles and spontaneous abortions, the selection criteria for NLRP7 sequencing in the studies described herein was widened and included patients with at least 1 HM (≧1 HM) or 3 SAs (≧3 SAs). NLRP7 mutation analyses in 135 unrelated patients with a spectrum of reproductive wastage are reported herewith. The highest frequency of NLRP7 mutations is found in patients with ≧2 HMs and the lowest is in patients with ≧3 SAs. A significant association between complete moles and the presence of at least one protein truncating mutation is demonstrated. Rare non-synonymous variants (NSVs) in NLRP7 confer genetic susceptibility for recurrent reproductive wastage. Patients with NLRP7 mutations and rare NSVs have variable degrees of placental abnormalities associated with increased perinatal morbidities.

As described herein, Applicants have identified a number of novel mutations in the NLRP7 gene in families having female members suffering from recurrent reproductive wastage including at least one recurrent hydatidiform mole or three spontaneous abortions.

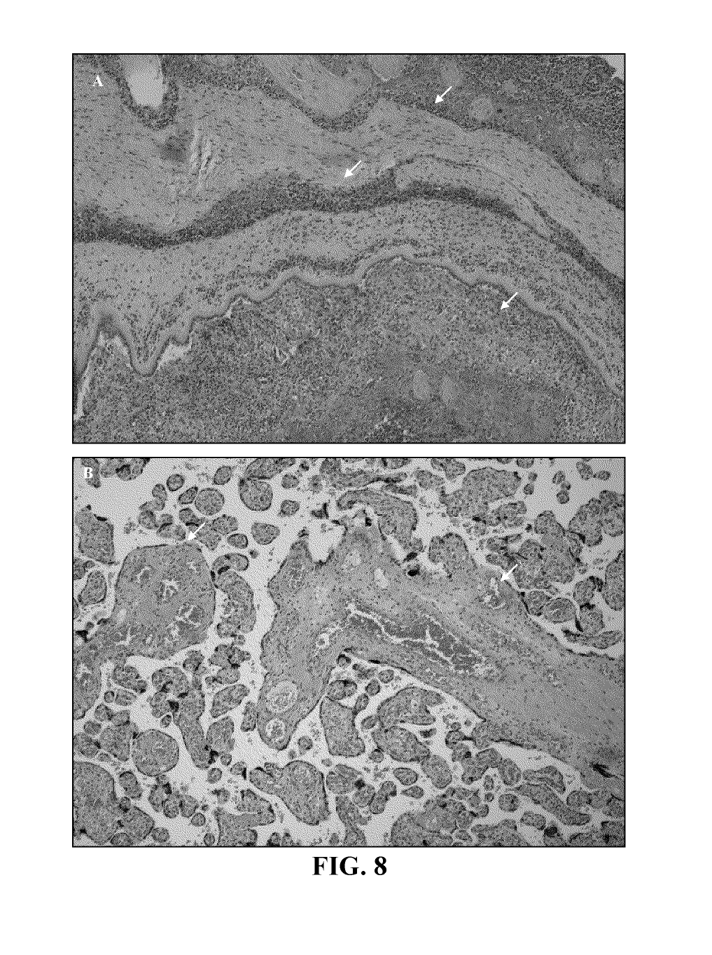

Accordingly, in a first aspect, the present invention provides a method for diagnosing a predisposition for reproductive wastage in a female subject, the method comprising detecting one or more alterations in the sequence of a NLRP7 nucleic acid or encoded polypeptide in a sample from said subject, relative to the sequence of a wild-type NLRP7 nucleic acid or encoded polypeptide, wherein said one or more alterations are one or more nonsynonymous mutations causing an amino acid changes at one or more positions corresponding to residues 250, 310, 311, 319, 340, 390, 413, 427, 430, 481, 487, 511, 659, 851, 872 and 931 in the NLRP7 polypeptide sequence of SEQ ID NO: 7 (FIG. 8C), wherein the presence of said alteration indicates that the female subject has a predisposition for recurrent reproductive wastage.

In an embodiment, the above-mentioned nonsynonymous mutation is a missense mutation or a nonsense mutation.

In an embodiment, the above-mentioned mutation causes: a Phe to Leu change at a position corresponding to residue 250 in the NLRP7 polypeptide sequence of SEQ ID NO: 7; a Gln to His or Gln to Arg change at a position corresponding to residue 310 in the NLRP7 polypeptide sequence of SEQ ID NO: 7; a Leu to Ile change at a position corresponding to residue 311 in the NLRP7 polypeptide sequence of SEQ ID NO: 7; a Val to Ile change at a position corresponding to residue 319 in the NLRP7 polypeptide sequence of SEQ ID NO: 7; a Met to Thr change at a position corresponding to residue 427 in the NLRP7 polypeptide sequence of SEQ ID NO: 7; an Ala to Thr change at a position corresponding to residue 481 in the NLRP7 polypeptide sequence of SEQ ID NO: 7; a Gly to Glu change at a position corresponding to residue 487 in the NLRP7 polypeptide sequence of SEQ ID NO: 7; a Lys to Arg change at a position corresponding to residue 511 in the NLRP7 polypeptide sequence of SEQ ID NO: 7; Glu to Gln or Gln to Lys change at a position corresponding to residue 340 in the NLRP7 polypeptide sequence of SEQ ID NO: 7; an Arg to His change at a position corresponding to residue 390 in the NLRP7 polypeptide sequence of SEQ ID NO: 7; an Arg to Trp change at a position corresponding to residue 413 in the NLRP7 polypeptide sequence of SEQ ID NO: 7; an Arg to His change at a position corresponding to residue 815 in the NLRP7 polypeptide sequence of SEQ ID NO: 7; a premature termination of the NLRP7 polypeptide at a position corresponding to residue 931 in the NLRP7 polypeptide sequence of SEQ ID NO: 7; a Tyr to Stop change (premature termination) at a position corresponding to residue 872 in the NLRP7 polypeptide sequence of SEQ ID NO: 7; or an Arg to Leu change at a position corresponding to residue 659 in the NLRP7 polypeptide sequence of SEQ ID NO: 7.

In an embodiment, the above-mentioned nonsynonymous mutation is:

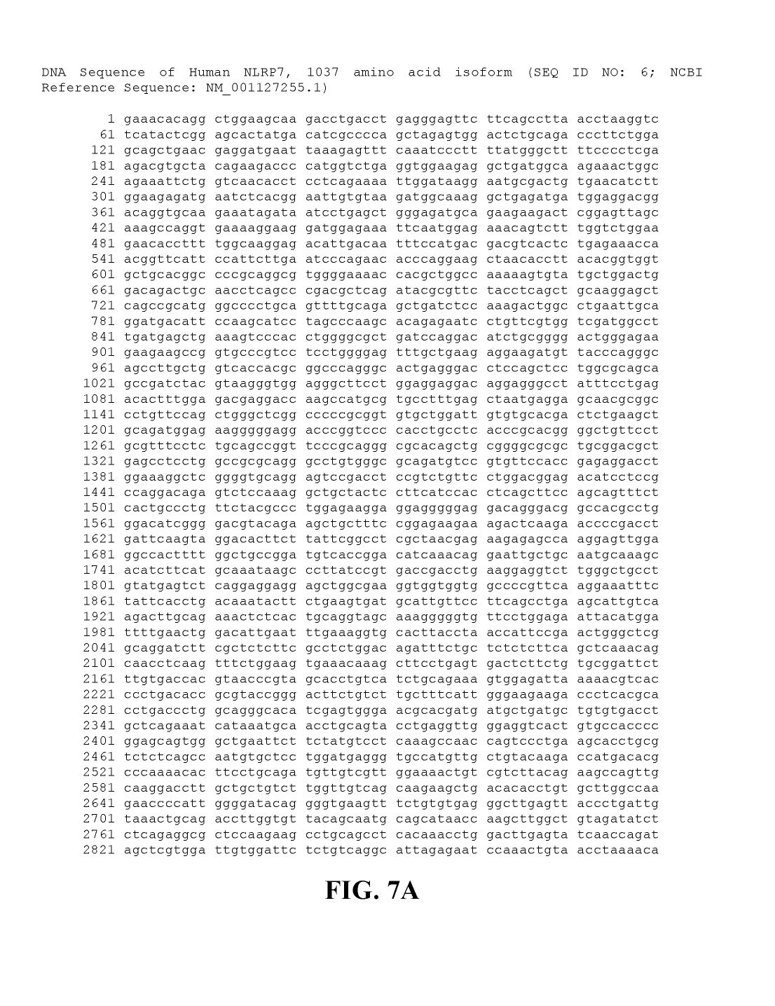

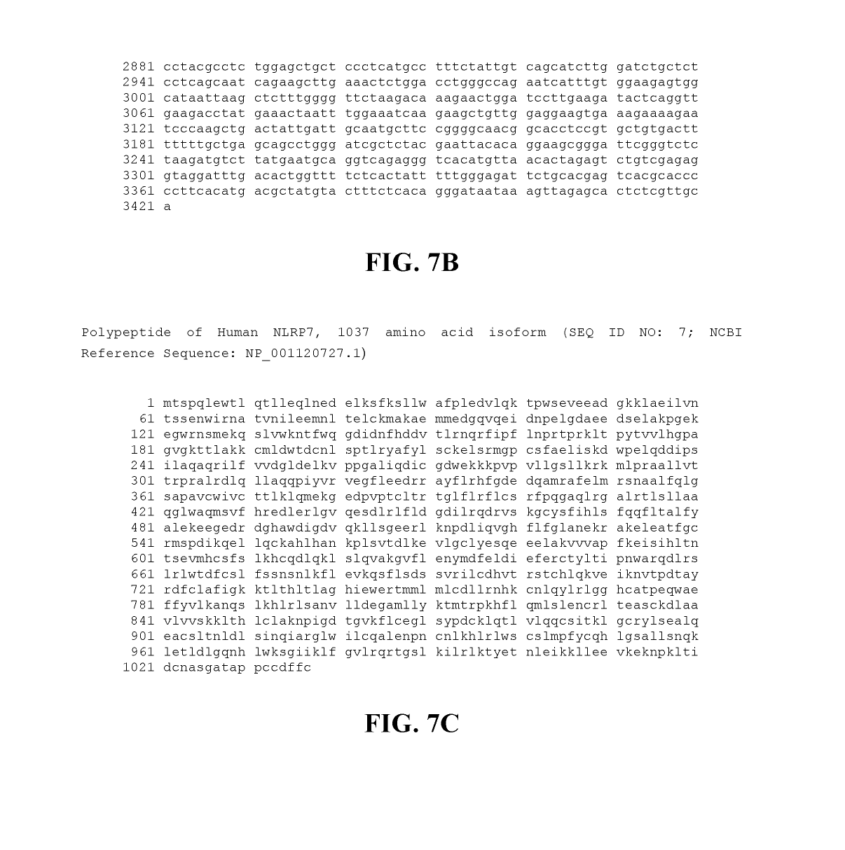

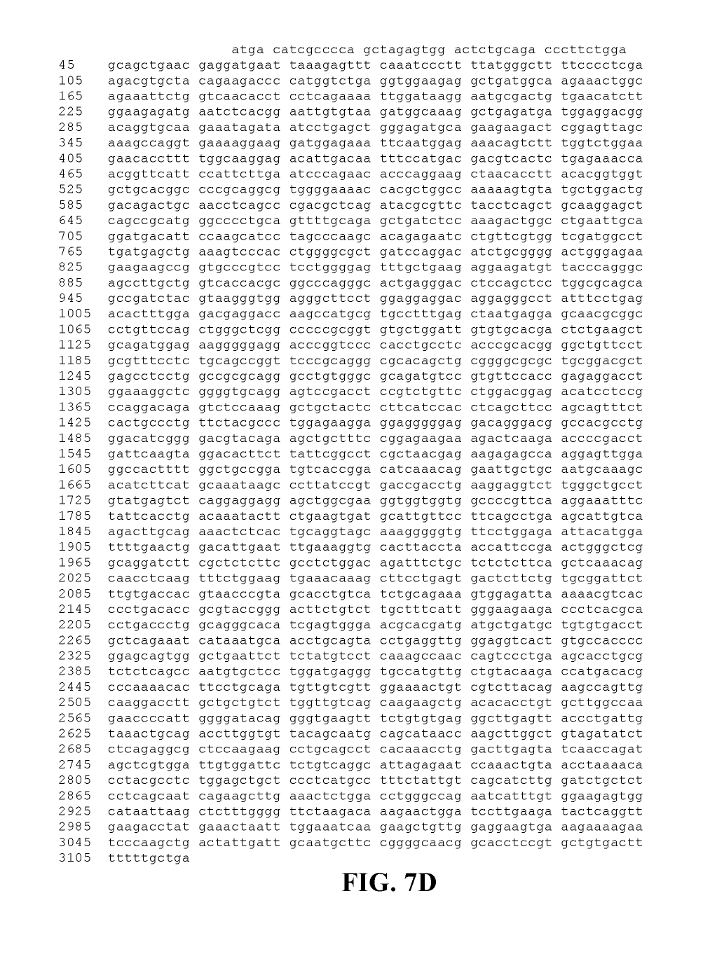

(i) a nucleotide substitution causing a Phe to Leu change at a position corresponding to residue 250 in the NLRP7 polypeptide sequence of SEQ ID NO: 7, in a further embodiment a C to A substitution at a position corresponding to nucleotide 750 in the NLRP7 nucleotide sequence of SEQ ID NO:8 (FIG. 7D);

(ii) a nucleotide deletion causing a Gln to His or Gln to Arg change at a position corresponding to residue 310 in the NLRP7 polypeptide sequence of SEQ ID NO: 7, in a further embodiment a deletion of a GC dinucleotide at positions corresponding to nucleotides 930 and 931 in the NLRP7 nucleotide sequence of SEQ ID NO:8 (FIG. 7D);

(iii) a nucleotide deletion and insertion causing a Glu to Gln change at a position corresponding to residue 340 in the NLRP7 polypeptide sequence of SEQ ID NO: 7, in a further embodiment a GAG to CAAAA substitution (i.e. a GAG deletion and a CAAAA insertion) at positions corresponding to nucleotides 1018 to 1020 in the NLRP7 nucleotide sequence of SEQ ID NO:8 (FIG. 7D);

(iv) a nucleotide substitution causing a Glu to Lys change at a position corresponding to residue 340 in the NLRP7 polypeptide sequence of SEQ ID NO: 7, in a further embodiment a G to A substitution at positions corresponding to nucleotide 1018 in the NLRP7 nucleotide sequence of SEQ ID NO:8 (FIG. 7D);

(v) a nucleotide substitution causing an Arg to His change at a position corresponding to residue 390 in the NLRP7 polypeptide sequence of SEQ ID NO: 7, in a further embodiment a G to A substitution at positions corresponding to nucleotide 1169 in the NLRP7 nucleotide sequence of SEQ ID NO:8 (FIG. 7D);

(vi) a nucleotide substitution causing an Arg to Trp change at a position corresponding to residue 413 in the NLRP7 polypeptide sequence of SEQ ID NO: 7, in a further embodiment a C to T substitution at positions corresponding to nucleotide 1237 in the NLRP7 nucleotide sequence of SEQ ID NO:8 (FIG. 7D);

(vii) a nucleotide substitution causing an Arg to His change at a position corresponding to residue 815 in the NLRP7 polypeptide sequence of SEQ ID NO: 7, in a further embodiment a G to AT substitution at positions corresponding to nucleotide 2444 in the NLRP7 nucleotide sequence of SEQ ID NO:8 (FIG. 7D);

(viii) a nucleotide deletion causing a premature termination of the NLRP7 polypeptide at a position corresponding to residue 931 in the NLRP7 polypeptide sequence of SEQ ID NO: 7, in a further embodiment a TG deletion at positions corresponding to nucleotides 2791 and 2792 in the NLRP7 nucleotide sequence of SEQ ID NO:8 (FIG. 7D);

(ix) a nucleotide substitution causing a Gln to Arg change at a position corresponding to residue 310 in the NLRP7 polypeptide sequence of SEQ ID NO: 7, in a further embodiment a A to G substitution at a position corresponding to nucleotide 929 in the NLRP7 nucleotide sequence of SEQ ID NO:8 (FIG. 7D);

(x) a nucleotide substitution causing a Leu to Ile change at a position corresponding to residue 311 in the NLRP7 polypeptide sequence of SEQ ID NO: 7, in a further embodiment a C to A substitution at a position corresponding to nucleotide 931 in the NLRP7 nucleotide sequence of SEQ ID NO:8 (FIG. 7D);

(xi) a nucleotide substitution causing a Val to Ile change at a position corresponding to residue 319 in the NLRP7 polypeptide sequence of SEQ ID NO: 7, in a further embodiment a G to A substitution at a position corresponding to nucleotide 955 in the NLRP7 nucleotide sequence of SEQ ID NO:8 (FIG. 7D);

(xii) a nucleotide substitution causing a Met to Thr change at a position corresponding to residue 427 in the NLRP7 polypeptide sequence of SEQ ID NO: 7, in a further embodiment a T to C substitution at a position corresponding to nucleotide 1280 in the NLRP7 nucleotide sequence of SEQ ID NO:8 (FIG. 7D);

(xiii) a nucleotide substitution causing a Phe to Leu change at a position corresponding to residue 430 in the NLRP7 polypeptide sequence of SEQ ID NO: 7, in a further embodiment a T to C substitution at a position corresponding to nucleotide 1288 in the NLRP7 nucleotide sequence of SEQ ID NO:8 (FIG. 7D);

(xiv) a nucleotide substitution causing a Ala to Thr change at a position corresponding to residue 481 in the NLRP7 polypeptide sequence of SEQ ID NO: 7, in a further embodiment a G to A substitution at a position corresponding to nucleotide 1441 in the NLRP7 nucleotide sequence of SEQ ID NO:8 (FIG. 7D);

(xv) a nucleotide substitution causing a Gly to Glu change at a position corresponding to residue 487 in the NLRP7 polypeptide sequence of SEQ ID NO: 7, in a further embodiment a T to C substitution at a position corresponding to nucleotide 1460 in the NLRP7 nucleotide sequence of SEQ ID NO:8 (FIG. 7D);

(xvi) a nucleotide substitution causing a Lys to Arg change at a position corresponding to residue 511 in the NLRP7 polypeptide sequence of SEQ ID NO: 7, in a further embodiment a A to G substitution at a position corresponding to nucleotide 1532 in the NLRP7 nucleotide sequence of SEQ ID NO:8 (FIG. 7D);

(xvii) a nucleotide substitution causing an Arg to Leu change at a position corresponding to residue 659 in the NLRP7 polypeptide sequence of SEQ ID NO: 7, in a further embodiment a G to T substitution at positions corresponding to nucleotide 1976 in the NLRP7 nucleotide sequence of SEQ ID NO:8 (FIG. 7D); or

(xviii) a nucleotide substitution causing a Tyr to Stop change at a position corresponding to residue 872 in the NLRP7 polypeptide sequence of SEQ ID NO: 7, in a further embodiment a C to A substitution at positions corresponding to nucleotide 2616 in the NLRP7 nucleotide sequence of SEQ ID NO:8 (FIG. 7D).

In an embodiment, the alteration is located in the NACHT domain of NLRP7, which corresponds to residues 172 to 491. In a further embodiment, the alteration is at a position corresponding to residue 250, 310, 311, 319, 340, 390, 413, 427, 430, 481 and/or 487 in the NLRP7 polypeptide sequence of SEQ ID NO: 7.

In an embodiment, the above-mentioned is an in vitro method.

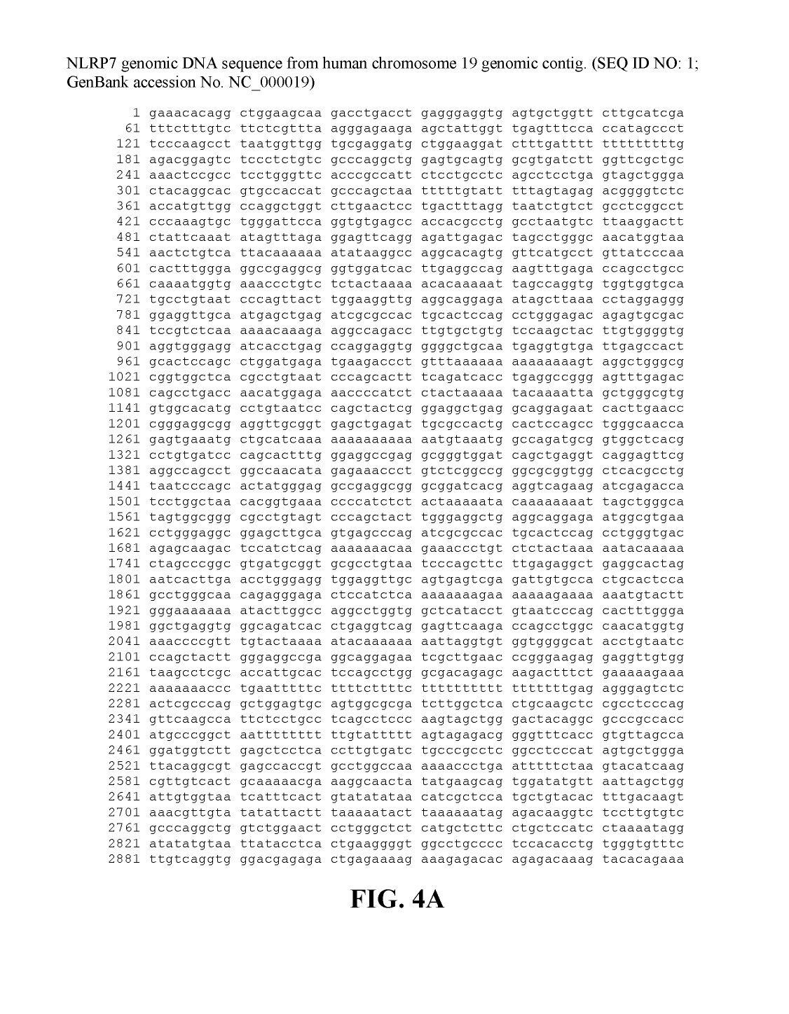

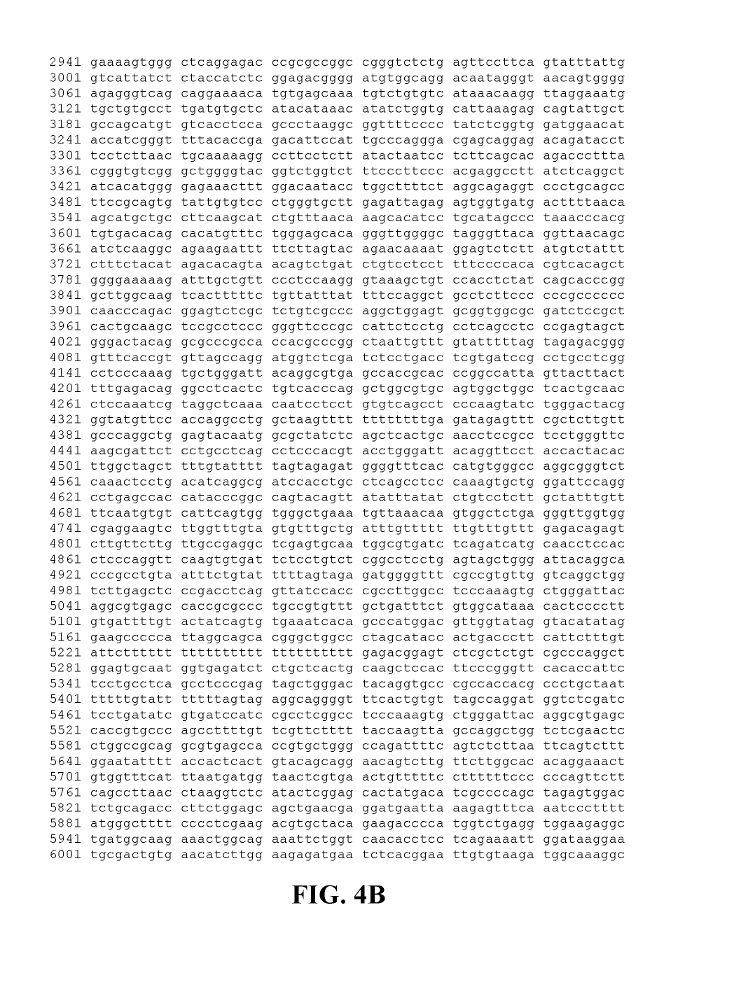

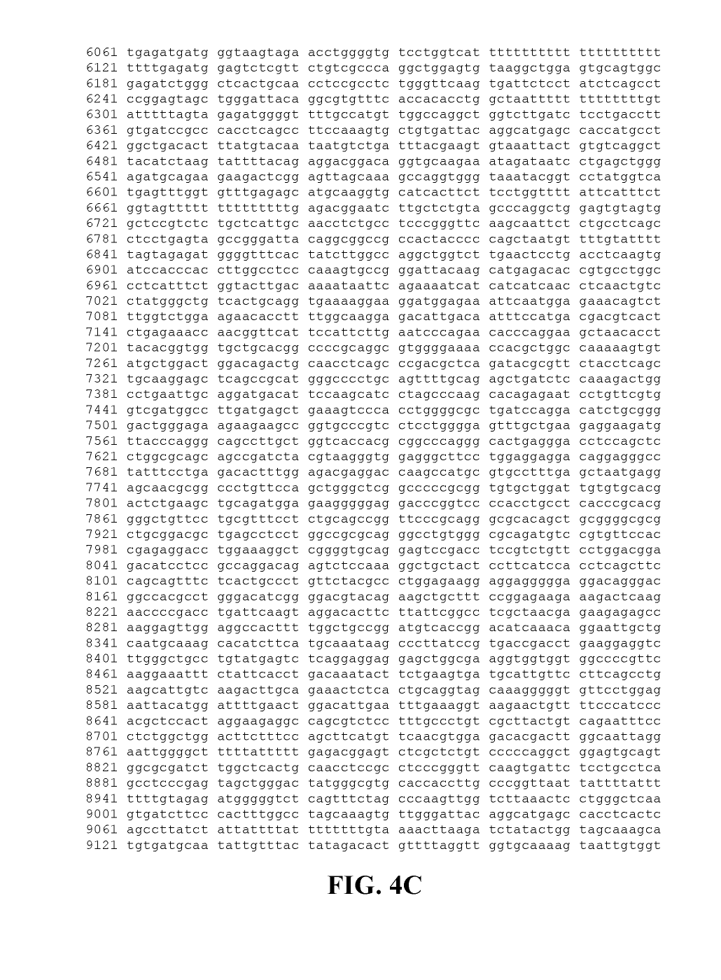

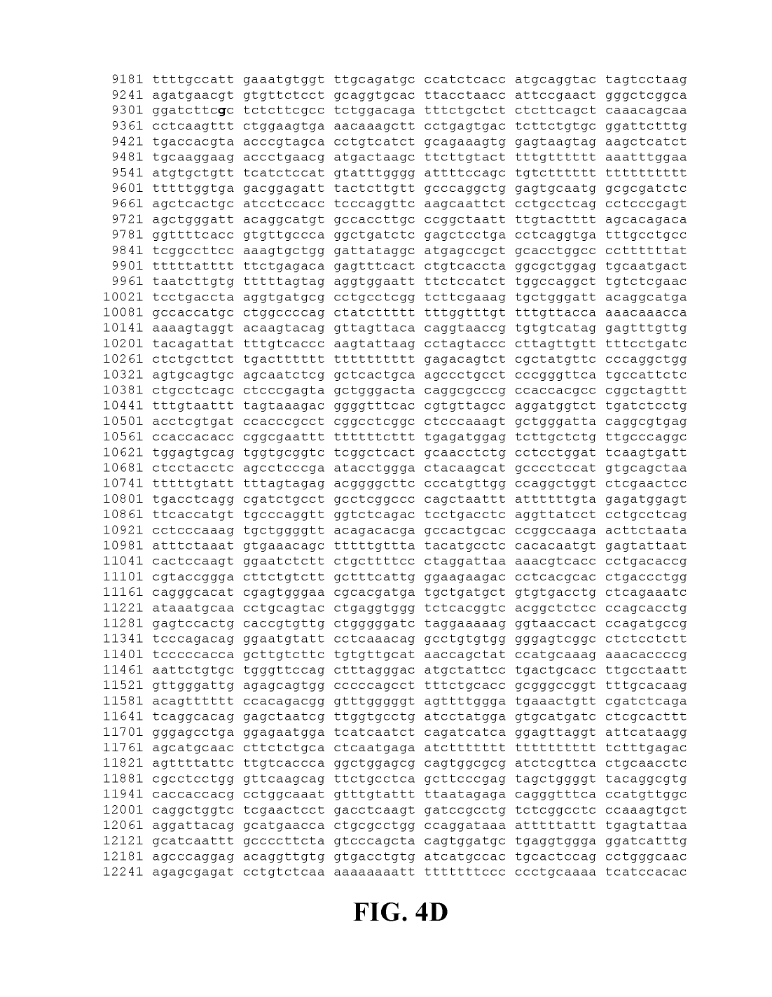

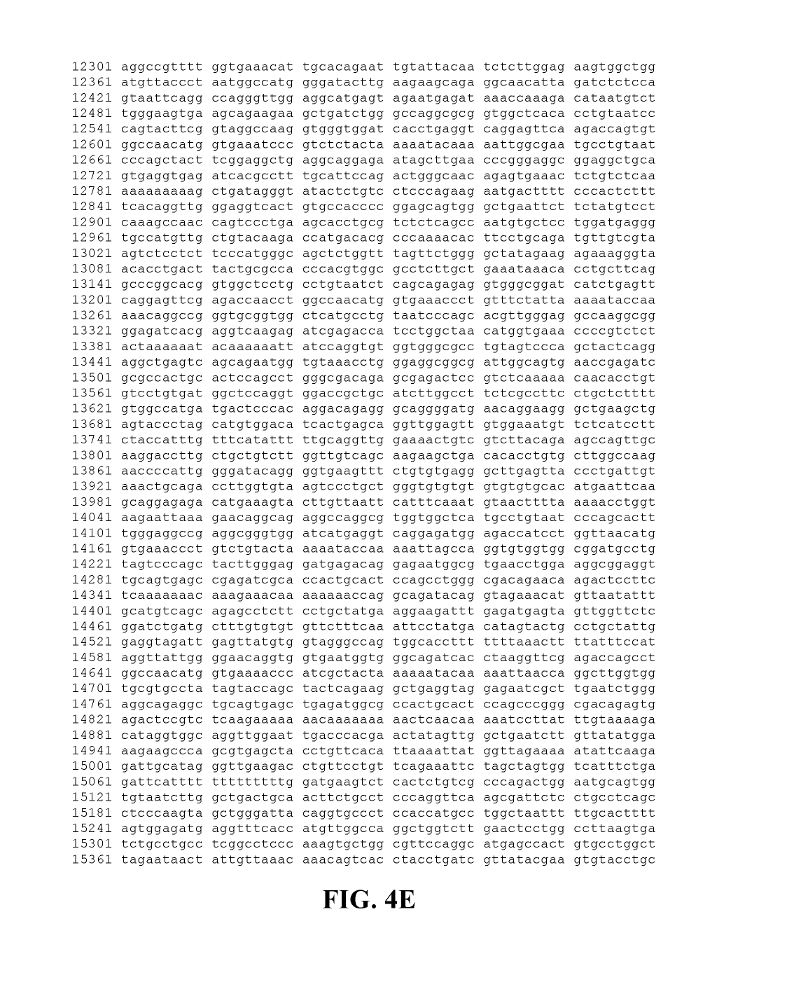

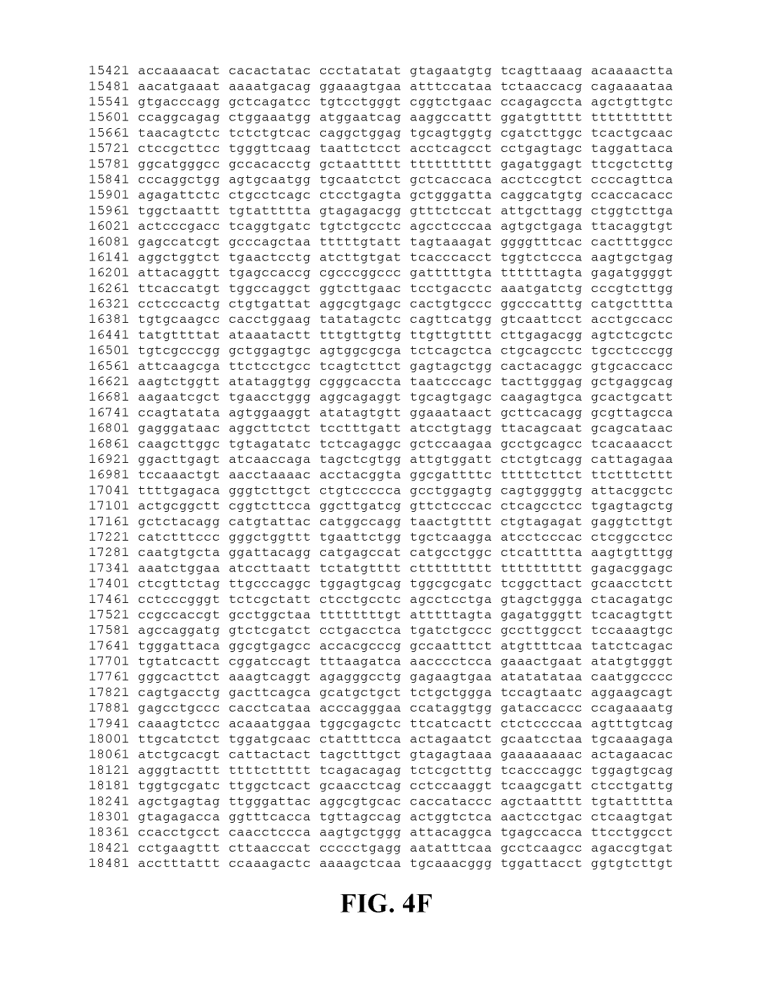

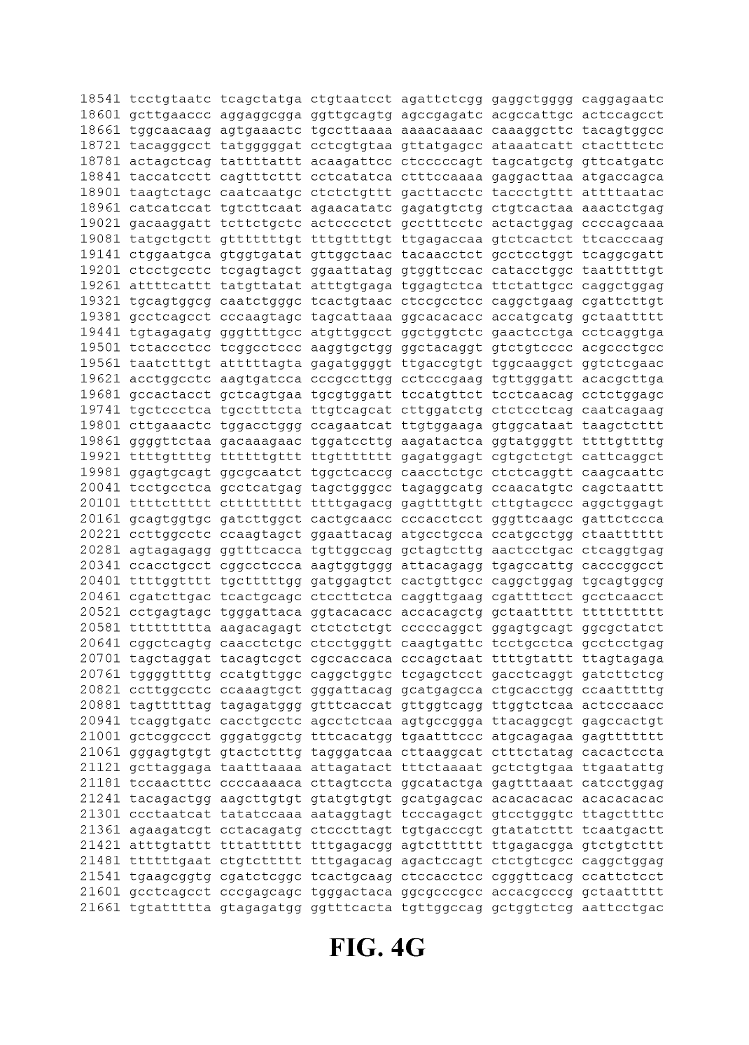

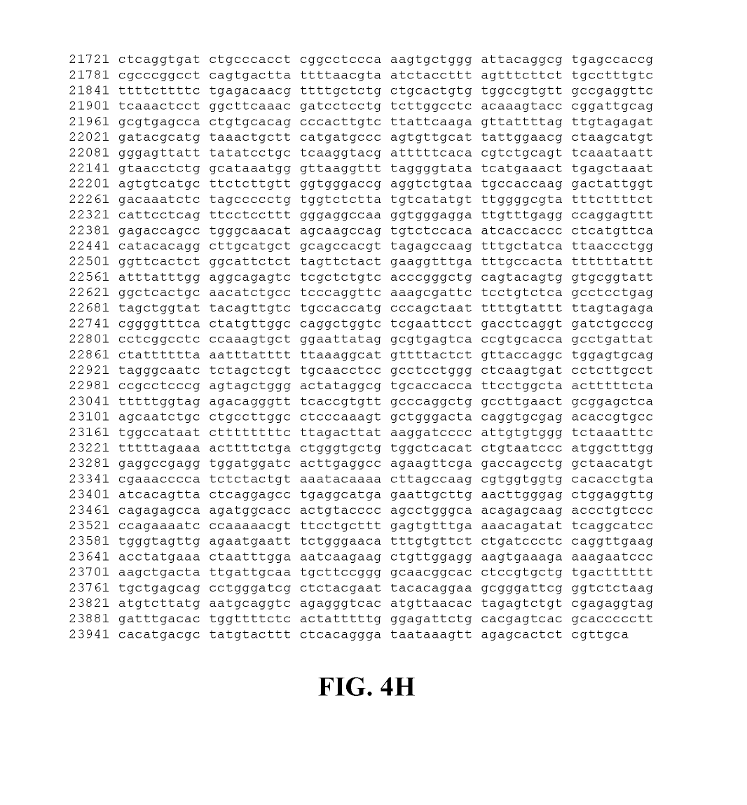

Wild-type or native NLRP7 nucleic acid and polypeptide sequences as used herein refer to sequences of NLRP7 nucleic acid or polypeptide found in samples from subjects not suffering from or not having a predisposition for recurrent reproductive wastage, and having NLRP7 activity. Examples of wild-type or native NLRP7 nucleic acid and polypeptide sequences are provided in FIGS. 4A to 4D and SEQ ID NOs: 1-8.

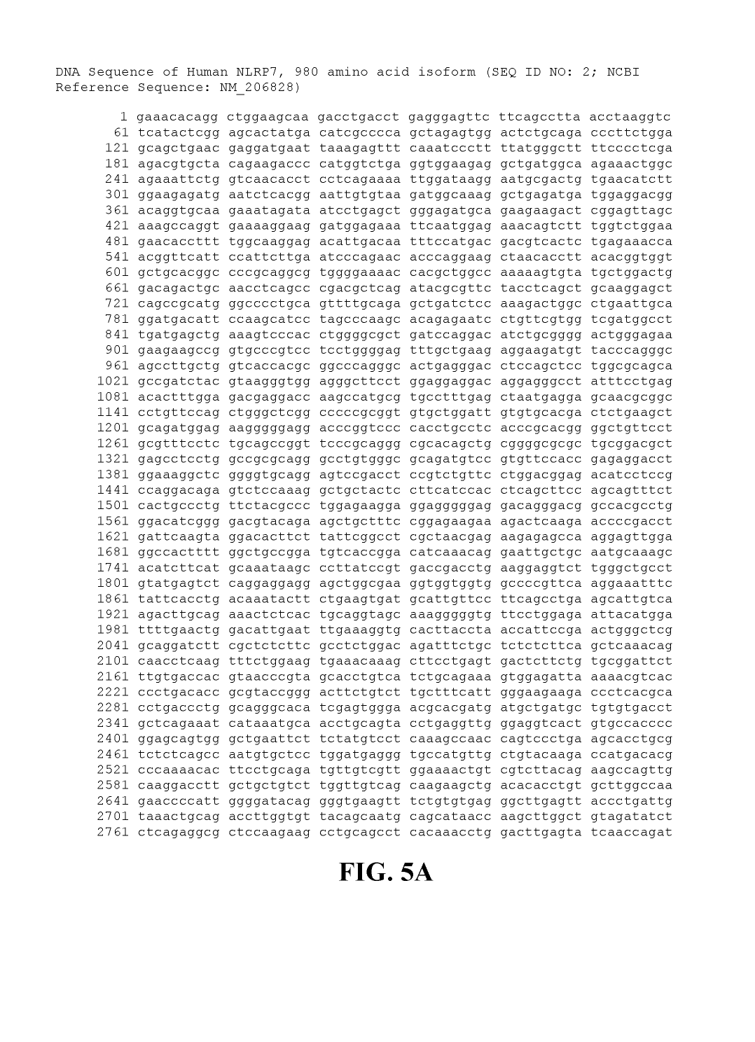

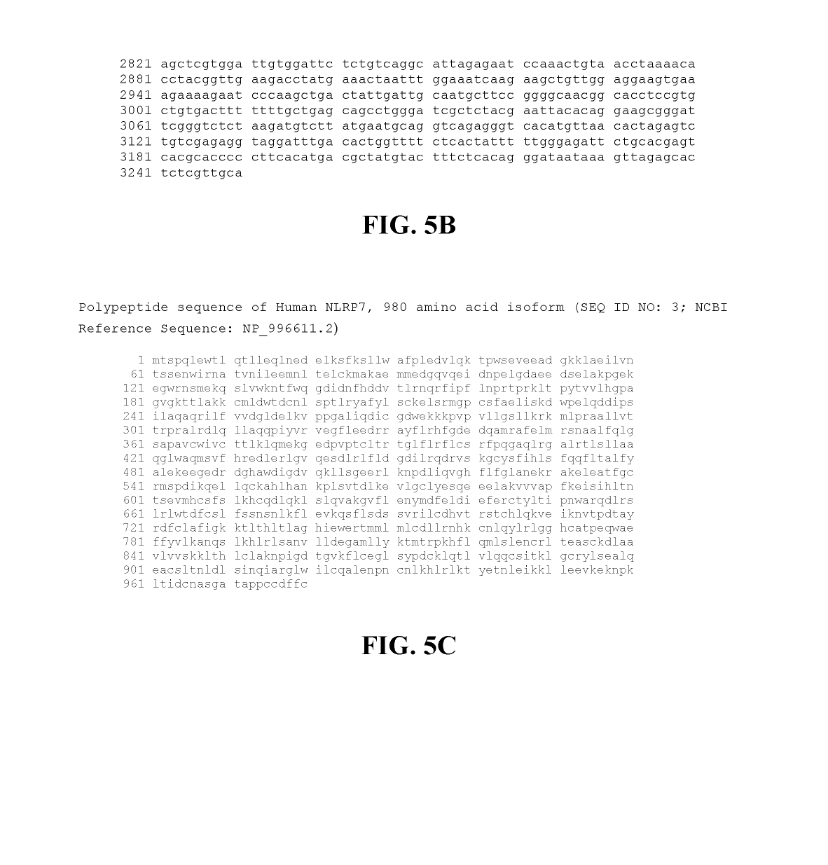

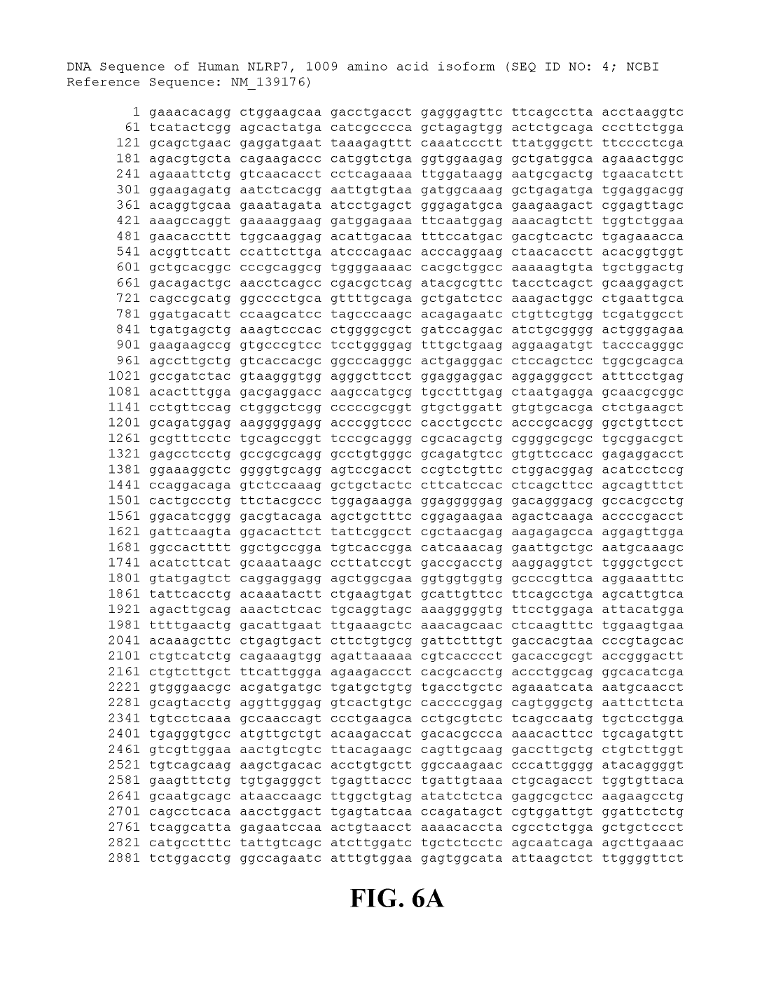

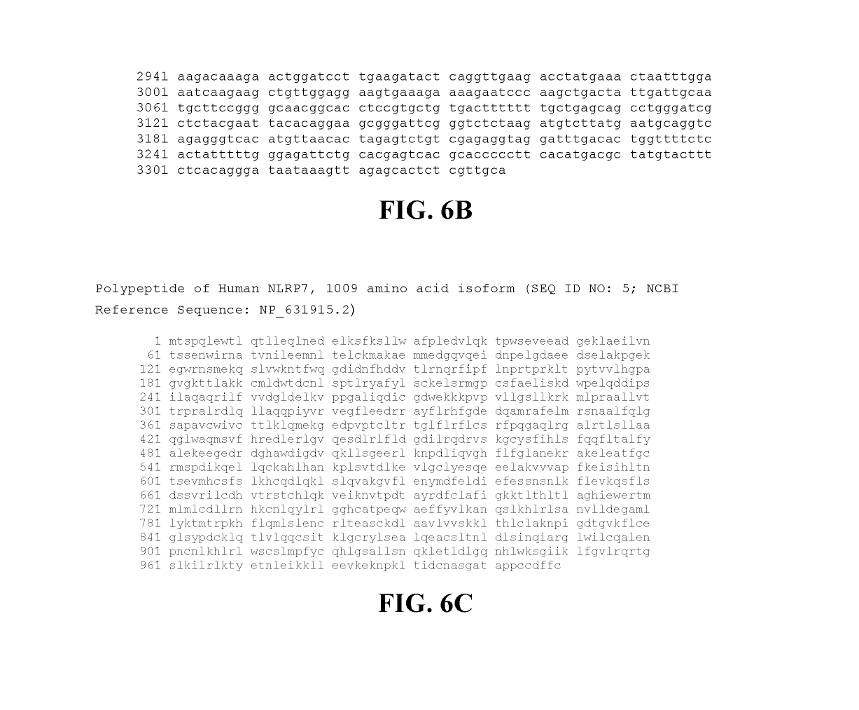

Nucleotide numbering for mutations and variants described herein uses cDNA numbering with +1 corresponding to the A of the ATG translation initiation codon in the reference human NLRP7 sequence, NCBI Reference Sequence: NM_{—}001127255.1 (FIG. 7D, SEQ ID NO:8, isoform 3, encoding the 1037 amino acids isoform). Similarly, amino acid numbering for mutations and variants described herein are based on the amino acid sequence of the native NLRP7 polypeptide depicted at FIG. 7C (SEQ ID NO: 7, 1037 amino acids isoform 3, NCBI Reference Sequence: NP_{—}001120727.1). It will be understood that nucleotide and amino acid numbering can thus be shifted in situations where the residues corresponding to those referred to herein (i.e., in reference to the numbering of FIG. 7C and FIG. 7D) are within a nucleic acid or polypeptide having more or fewer nucleotide or amino acids 5′ or N-terminal to the region(s) where these residues reside (e.g., a different isoform), relative to the reference NLRP7 sequences (FIG. 7C and FIG. 7D), thereby resulting in different nucleotide or amino acid numbering. The corresponding positions may be easily identified, for example by aligning the sequence of a given NLRP7 nucleic acid or polypeptide with that depicted in FIG. 7C or FIG. 7D (e.g., using a software for sequence alignment such as Clustal W). For example, the positions corresponding to positions 250, 310, 340, 390, 413, 659, 851, 872 and 931 of the NLRP7 polypeptide sequence of SEQ ID NO: 7 (FIG. 7C) in other isoforms of human NLRP7 polypeptides (FIGS. 5C and 6C) are depicted in Table A below.

TABLE A
Positions corresponding to positions 250, 310, 340, 390, 413, 659,
851, 872 and 931 of the NLRP7 polypeptide sequence of SEQ
ID NO: 7 (FIG. 7C) in other isoforms of human NLRP7 polypeptides

| | Position corresponding to positions . . . of SEQ ID NO: | | | | | | | | |
|---|---|---|---|---|---|---|---|---|---|
| | 7 (FIG. 7C) | | | | | | | | |
| Isoform | 250 | 310 | 340 | 390 | 413 | 659 | 851 | 872 | 931 |
| Isoform | 250 | 310 | 340 | 390 | 413 | 659 | 851 | 872 | 931 |
| 2 (FIG. | | | | | | | | | |
| 5C) | | | | | | | | | |
| Isoform | 250 | 310 | 340 | 390 | 413 | — | 823 | 844 | 903 |
| 1 (FIG. | | | | | | | | | |
| 6C) | | | | | | | | | |

— = Isoform 1 uses an alternate in-frame, and the region corresponding to residues 644 to 671 of SEQ ID NO: 7 is lacking.

Similarly, the positions corresponding to positions 750, 930-931, 1018-1020, 1169, 1237, 1976, 2444, 2616 and 2791-2792 of the NLRP7 cDNA sequence of SEQ ID NO: 8 (FIG. 8D) in isoforms 1 to 3 of human NLRP7 nucleic acids (FIGS. 5A-B, 6A-B and 7A-B) and in the human NLRP7 gene sequence (FIGS. 4A-H) are depicted in Table B below.

TABLE B
Positions corresponding to positions 750, 930-931, 1018-1020, 1169, 1237,
1976, 2444, 2626 and 2791-2792 of the NLRP7 cDNA sequence of SEQ ID NO: 8 (FIG.
7D) in isoforms 1 to 3 of human NLRP7 nucleic acids (FIGS. 5A-B, 6A-B and 7A-B)
and in the human NLRP7 gene (FIGS. 4A-H)

| Isoform/ | Position corresponding to positions . . . of SEQ ID NO: 8 (FIG. 7D) | | | | | | | | |
|---|---|---|---|---|---|---|---|---|---|
| gene | 750 | 930-931 | 1018-1020 | 1169 | 1237 | 1976 | 2444 | 2626 | 2791-2792 |
| Isoform | 826 | 1006-1007 | 1094-1096 | 1245 | 1313 | 2054 | 2520 | 2702 | 2867-68 |
| 2 (FIGS. | | | | | | | | | |
| 5A-B) | | | | | | | | | |
| Isoform | 826 | 1006-1007 | 1094-1096 | 1245 | 1313 | — | 2436 | 2618 | 2783-2784 |
| 1 (FIGS. | | | | | | | | | |
| 6A-B) | | | | | | | | | |
| Isoform | 826 | 1006-1007 | 1094-1096 | 1245 | 1313 | 2054 | 2520 | 2702 | 2867-68 |
| 3 (FIGS. | | | | | | | | | |
| 7A-B) | | | | | | | | | |
| Gene | 7437 | 7618-7619 | 7705-7707 | 7856 | 7924 | 9309 | 12990 | 13921 | 16988-16989 |
| (FIGS. | | | | | | | | | |
| 5A-H | | | | | | | | | |

— = Isoform 1 uses an alternate in-frame, and the region encompassing this nucleotide is lacking.

An “altered” or “mutated” NLRP7 nucleic acid or polypeptide as used herein refers to a nucleic acid or polypeptide having a different nucleotide or amino acid from the native nucleic acid or protein at at least one of the nucleotide or amino acid or positions described more fully in the specification, and which is associated with a predisposition for reproductive wastage (e.g., an increased risk of having or developing the condition relative to a subject not having the altered or mutated NLRP7 nucleic acid or polypeptide).

“Alteration” as used herein in respect of a nucleotide or polypeptide sequence refers to any type of mutation or change relative to the corresponding wild-type nucleotide or polypeptide sequence, including deletions, insertions, substitutions and point mutations. In an embodiment, the above-mentioned alteration is a non-synonymous mutation, for example resulting in an amino acid change/substitution, the creation of a stop codon, or a frameshift.

In embodiments, the detection of the alteration in the sequence of a NLRP7 nucleic acid may be performed at the gene or mRNA level. In embodiments, the alteration may be determined at the polypeptide level.

In an embodiment, the subject is a female mammal, e.g., a human female subject.

In an embodiment, the female subject is undergoing, or is a candidate for, assisted reproductive technologies (ART). Examples of ART include in vitro fertilization, intracytoplasmic sperm injection (ICSI), cryopreservation, and intrauterine insemination (IUD.

In embodiments, the subject may be heterozygous (i.e., where one allele of the NLRP7 gene comprises the alteration) or homozygous (i.e., where both alleles of the NLRP7 gene comprise the alteration) for the alteration. As described herein, the presence of the alteration in both alleles is associated with/indicative of a higher risk of or greater predisposition to recurrent reproductive wastage.

In embodiments, the recurrent reproductive wastage is any type of recurrent and non-recurrent spontaneous abortion, blighted ovum, stillbirth associated or not with preeclampsia, ectopic pregnancy, hydatidiform mole (e.g., complete or partial hydatidiform mole), placental abnormalities such as chorioamnionitis, chorangiosis, placental hemorrhage, molar pregnancy, biparental molar pregnancy, androgenetic molar pregnancy, triploid molar pregnancies, gestational trophoplastic disease, persistent trophoblastic disease, gestational trophoblastic tumor/neoplasia, invasive mole, elective termination and choriocarcinoma.

In a further embodiment, recurrent reproductive wastage is spontaneous abortion, blighted ovum, hydatidiform mole (e.g., complete or partial hydatidiform mole), choriocarcinoma, gestational trophoblastic neoplasia, placental abnormalities such as chorioamnionitis, chorangiosis, placental hemorrhage.

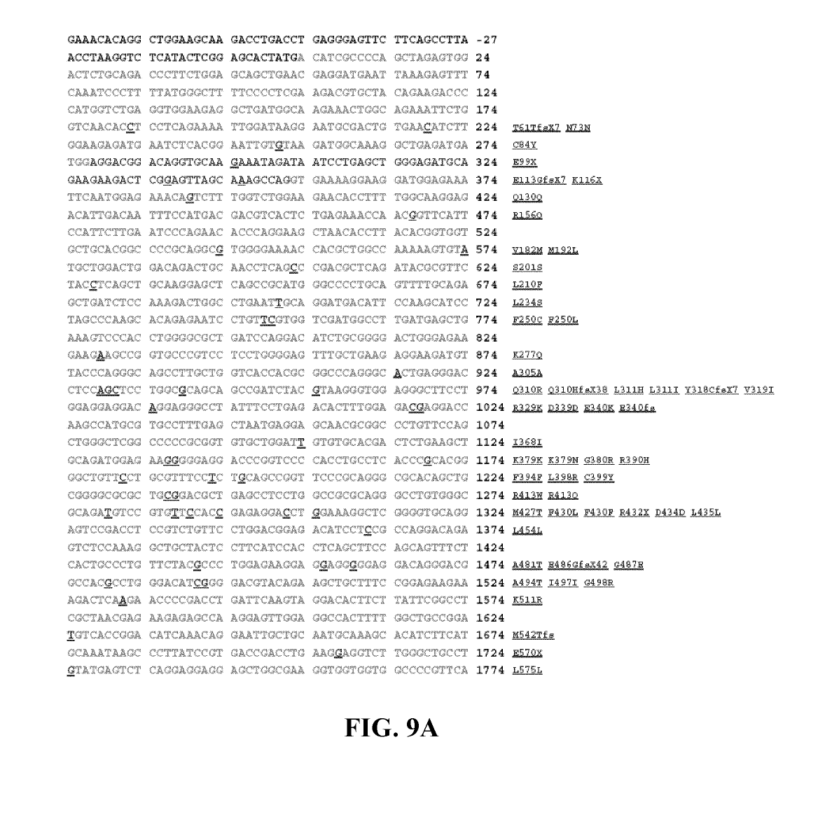

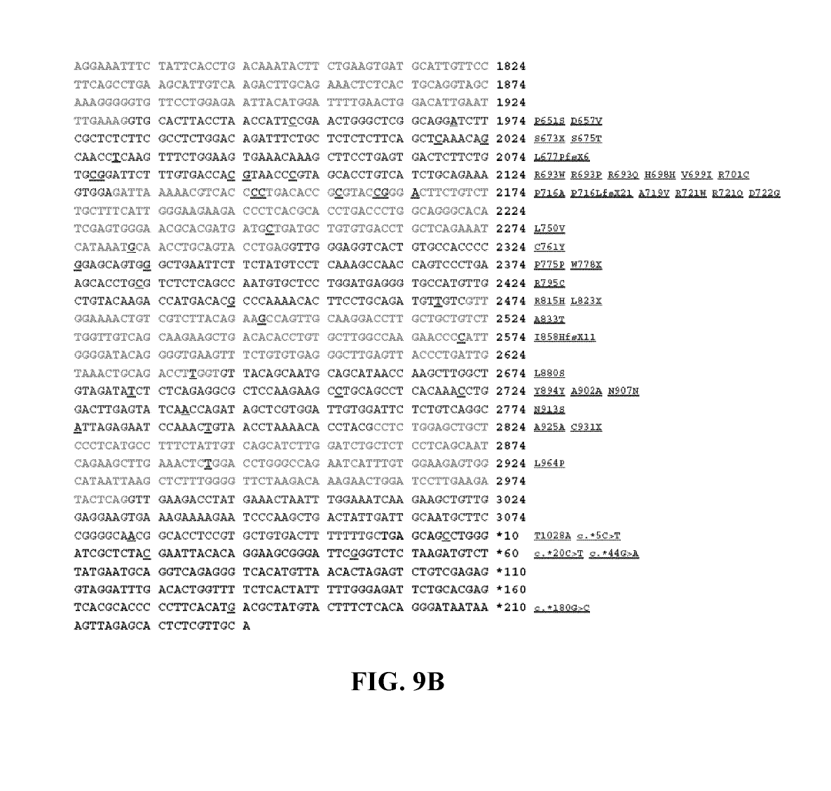

The detection of any combination of the above-noted alterations may also be used in the methods of the invention. Also, the further detection of one or more additional NLRP7 mutations/alterations known to be linked to a predisposition to recurrent reproductive wastage, such as those described in references 15 to 22 may also be used in the methods of the invention. Several additional NLRP7 mutations/alterations known to be linked to a predisposition to recurrent reproductive wastage (e.g., reproductive wastage) are depicted in FIGS. 9A and 9B. The presence of more than one NLRP7 alterations in the sample may be indicative of a higher risk or higher predisposition to having reproductive wastage.

In embodiments, the nucleic acid comprises an altered NLRP7 nucleotide sequence comprising the above-noted one or more alterations as well as one or more further alterations associated with altered splicing of a NLRP7 transcript, such as altered splicing of exon 3, exon 7, or both, of said NLRP7 gene.

In an embodiment, the one or more further alterations occurs at a splice donor site, such as at the splice donor site at the boundary of exon 3 and intron 3, the splice donor site at the boundary of exon 7 and intron 7, or both, of the NLRP7 gene.

In an embodiment, the one or more further alteration results in a loss of a cleavage site for a restriction endonuclease (e.g., BstNI) in the NLRP7 gene.

In an embodiment, the one or more further alterations is at an amino acid position within the NLRP7 polypeptide selected from position 693, 399, 379, 99 and 657 of the NLRP7 polypeptide.

In embodiments, the one or more further alterations is selected from a substitution of the C corresponding to the first position of the codon for Arg 693 of the NLRP7 polypeptide and a substitution of the G corresponding to the second position of the codon for Arg 693 of the NLRP7 polypeptide. In further embodiments, the one or more further alterations is selected from a substitution of Arg 693 with Trp (R693W).

In further embodiments, the one or more further alterations is selected from (a) a substitution of Cys 399 with Tyr (C399Y); (b) a substitution of Lys 379 with Asn (K379N); (c) a substitution of the codon for Glu 99 with a stop codon (E99X); and (d) a substitution of Asp 657 with Val (D657V).

In embodiments, the one or more further alterations is selected from: (a) a substitution of G with A at the splice donor site at the boundary of exon 3 and intron 3 (IVS3+1G>A); (b) a substitution of G with A at the splice donor site at the boundary of exon 7 and intron 7 (IVS7+1G>A); (c) a substitution of C with T corresponding to the first position of the codon for Arg 693 of the NLRP7 polypeptide; (d) a substitution of G with A corresponding to the second position of the codon for Cys 84 of the NLRP7 polypeptide; (e) a substitution of G with A corresponding to the second position of the codon for Cys 399 of the NLRP7 polypeptide; (f) a substitution of G with C corresponding to the third position of the codon for Lys 379 of the NLRP7 polypeptide; (g) a substitution of G with T corresponding to the first position of the codon for Glu 99 of the NLRP7 polypeptide; and (h) a substitution of A with T corresponding to the second position of Asp 657 of the NLRP7 polypeptide.

Further, the above-mentioned method may further comprise selection of a prophylactic or therapeutic course of action in accordance with the detected alteration. For example, the method of the present invention may be used in preimplementation genetic diagnosis (PGD)/assisted reproductive technologies (ART). Patients in which several mutations in NLRP7 are detected, or in which NLRP7 mutations are present in two alleles, which typically have a worst reproductive outcomes from natural conceptions as compared to patients with only one NLRP7 mutations (or in which NLRP7 mutations are present in only one allele) would benefit from ovum donation in order to increase the likelihood of having a normal pregnancy (see Example 8 and Qian J et al., Mol Hum Reprod. 2011 October; 17(10):612-9. Epub 2011 Apr. 19). Also, patients with one ore more mutations in the NACHT domain have higher rates of postzygotic aneuploidies and mosaicisms than the average observed in women undergoing ART meaning that these patients would benefit from preimplantation genetic diagnosis (PGD) for aneuploidies to transfer to them diploid embryos and increase their chances of having normal pregnancies.

Accordingly, in another aspect, the present invention provides a method for determining the appropriate course of action in a female subject undergoing, or who is a candidate for, assisted reproductive technologies (ART), the method comprising (1) determining a predisposition for reproductive wastage of said subject using the method described herein; and (2) determining the appropriate course of action based on said predisposition. In an embodiment, if one or more NLRP7 mutations are present in both alleles, the appropriate course of action comprises ovum donation from a healthy donor (i.e. a donor not having an alteration in NLRP7).

In another aspect, the present invention provides a method for determining whether a female subject could benefit from assisted reproductive technologies (ART), the method comprising (1) determining a predisposition for reproductive wastage of said subject using the method described herein; and (2) determining whether the female subject could benefit from assisted reproductive technologies based on said predisposition. In an embodiment, the presence of one or more NLRP7 mutations is indicative that the female subject could benefit from ART. In a further embodiment, the presence of one or more NLRP7 mutations in both alleles is indicative that the female subject could benefit from ART. In an embodiment, at least one of the one or more NLRP7 mutations is in the NACHT domain. In a further embodiment, all of the NLRP7 mutations are in the NACHT domain. In an embodiment, the ART comprises ovum donation from a healthy donor (i.e. a donor not having an alteration in NLRP7).

The detection of the alteration(s) in NLRP7 may be useful for pre-implantation genetic diagnosis (PGD), for determining whether an embryo or oocyte carries a genetic predisposition for a reproductive condition. In another aspect, the present invention thus provides a method for determining whether an embryo or oocyte carries a genetic predisposition for a reproductive condition, the method comprising detecting one or more of the alterations in the sequence of a NLRP7 nucleic acid or encoded polypeptide according to the method described herein in said embryo or oocyte, wherein the presence of said alteration is indicative that said embryo or oocyte carries a genetic predisposition for a reproductive condition.

PGD is performed prior to implantation of embryos. The patient's oocytes are generally fertilized in vitro and the embryos kept in culture until the diagnosis is established. It also involves performing a biopsy on these embryos in order to obtain material (genetic material, nucleic acids, polypeptides) on which to perform the diagnosis. The diagnosis itself can be carried out using several techniques such as amplification-based methods (PCR, whole genome amplification), comparative genomic hybridization or Fluorescent in situ hybridization (FISH), depending on the nature of the studied condition.

In various embodiments, the above-noted sample can be from any source that contains genomic DNA, RNA, and/or proteins, for example a tissue or body fluid from the subject, such as blood, serum, immune cells (e.g., lymphocytes), epithelia, endometrial, uterine biopsies or oocytes. A test sample from fetal cells or tissue can be obtained by appropriate methods such as by amniocentesis or chorionic villus sampling. The sample may be subjected to commonly used isolation and/or purification techniques for enrichment in nucleic acids (genomic DNA, mRNA) and/or proteins.

The above noted alteration may be detected by a number of methods which are known in the art. Examples of suitable methods for detecting alterations at the nucleic acid level include sequencing of the NLRP7 nucleic acid sequence; hybridization of a nucleic acid probe capable of specifically hybridizing to a NLRP7 nucleic acid sequence comprising the alteration and not to (or to a lesser extent to) a corresponding wild-type NLRP7 nucleic acid sequence (under comparable hybridization conditions, such as stringent hybridization conditions); restriction fragment length polymorphism analysis (RFLP); Amplified fragment length polymorphism PCR (AFLP-PCR); amplification of a nucleic acid fragment comprising a NLRP7 nucleic acid sequence using a primer specific for the alteration, wherein the primer produces an amplified product if the alteration is present and does not produce the same amplified product when a corresponding wild-type NLRP7 nucleic acid sequence is used as a template for amplification (e.g., allele-specific PCR). Other methods include in situ hybridization analyses and single-stranded conformational polymorphism analyses.

Examples suitable methods for detecting alterations at the polypeptide level include sequencing of the NLRP7 polypeptide; digestion of the NLRP7 polypeptide followed by mass spectrometry or HPLC analysis of the peptide fragments, wherein the alteration of the NLRP7 polypeptide results in an altered mass spectrometry or HPLC spectrum as compared to wild-type NLRP7 polypeptide; and immunodetection using an immunological reagent (e.g., an antibody, a ligand) which exhibits altered immunoreactivity with a NLRP7 polypeptide comprising the alteration relative to a corresponding wild-type NLRP7 polypeptide. Immunodetection can measure the amount of binding between a polypeptide molecule and an anti-protein antibody by the use of enzymatic, chromodynamic, radioactive, magnetic, or luminescent labels which are attached to either the anti-protein antibody or a secondary antibody which binds the anti-protein antibody. In addition, other high affinity ligands may be used. Immunoassays which can be used include e.g. ELISAs, Western blots, and other techniques known to those of ordinary skill in the art (see Harlow and Lane, Antibodies: A Laboratory Manual, Cold Spring Harbor Laboratory Press, Cold Spring Harbor, N.Y., 1999 and Edwards R, Immunodiagnostics: A Practical Approach, Oxford University Press, Oxford; England, 1999). Methods to generate antibodies exhibiting altered immunoreactivity with a NLRP7 polypeptide comprising the alteration relative to a corresponding wild-type NLRP7 polypeptide are described in more detail below.

All these detection techniques may also be employed in the format of microarrays, protein-arrays, antibody microarrays, tissue microarrays, electronic biochip or protein-chip based technologies (see Schena M., Microarray Biochip Technology, Eaton Publishing, Natick, Mass., 2000).

Further, NLRP7 nucleic acid-containing sequences may be amplified using known methods (e.g., polymerase chain reaction [PCR]) prior to or in conjunction with the detection methods noted herein. The design of various primers for such amplification is known in the art.

The detection methods herein may also be performed in an assay utilizing a substrate having detection reagents attached thereto at discrete locations, such as a nucleic acid microarray. The invention further provides a substrate comprising an isolated altered NLRP7 nucleic acid described herein attached thereto.

The invention further provides an oligonucleotide (e.g., a probe or primer), capable of specifically hybridizing to the altered NLRP7 nucleotide sequence and not to (or to a lesser extent to) a corresponding wild-type NLRP7 nucleic acid sequence (under comparable hybridization conditions). Such hybridization may be under moderately stringent, or preferably stringent, conditions, as noted below. Such an oligonucleotide or plurality thereof may in embodiments be attached to a solid substrate, as noted above. Such oligonucleotides may be used to specifically detect the presence of an altered NLRP7 nucleic acid in a sample. In an embodiment, such oligonucleotide hybridizes to a portion of the NLRP7 nucleic acid comprising one or more of the alterations noted above (e.g., a portion comprising an alteration at nucleotides corresponding to nucleotides 750, 929-931, 955, 1018-1020, 1169, 1237, 1280, 1288, 1441, 1460, 1532, 1976, 2444, 2626 and/or 2791-2792 of the sequence of SEQ ID NO: 8 (FIG. 7D). In embodiment, such oligonucleotide comprises one or more mutations corresponding to the above-noted alterations in NLRP7 (or to the complement thereof).

The invention further provides (a) nucleic acid primer(s) (e.g. an amplification pair) specific for the alteration, wherein the primer(s) produce(s) an amplified product if the alteration is present and does not produce the same amplified product (or produces a different signature of amplified products) when a corresponding wild-type NLRP7 nucleic acid sequence is used as a template for amplification. The terminology “amplification pair” refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification. As commonly known in the art, the oligos are designed to bind to a complementary sequence under selected conditions. Accordingly, the invention further provides an amplification pair capable of amplifying an altered NLRP7 nucleic acid, a wild-type NLRP7 nucleic acid, or a fragment of an altered NLRP7 nucleic acid or a wild-type NLRP7 nucleic acid.

Oligonucleotide probes or primers of the present invention may be of any suitable length, depending on the particular assay format and the particular needs and targeted sequences employed. In general, the oligonucleotide probes or primers are at least 12 nucleotides in length, preferably from about 12 to about 100 nucleotides in length, in embodiments from about 12 to about 50, from about 12 to about 30, or from about 15 to about 24 nucleotides in length. They may be adapted to be especially suited to a chosen nucleic acid amplification system. As commonly known in the art, the oligonucleotide probes and primers can be designed by taking into consideration the melting point of hybridization thereof with its targeted sequence (see below and in Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

Probes or primers of the invention can be utilized with naturally occurring sugar-phosphate backbones as well as modified backbones including phosphorothioates, dithionates, alkyl phosphonates and α-nucleotides and the like. Modified sugar-phosphate backbones are generally taught by Miller, 1988, Ann. Reports Med. Chem. 23:295 and Moran et al., 1987, Nucleic Acids Res., 14:5019. Probes or primers of the invention can be constructed of either ribonucleic acid (RNA) or deoxyribonucleic acid (DNA), and preferably of DNA.

The types of detection methods in which probes can be used include

Southern blots (DNA detection), dot or slot blots (DNA, RNA), and Northern blots (RNA detection). Although less preferred, labeled proteins could also be used to detect a particular nucleic acid sequence to which it binds.

Although the present invention is not specifically dependent on the use of a label for the detection of a particular nucleic acid sequence, such a label might be beneficial, by increasing the sensitivity of the detection. Furthermore, it enables automation (the same can also be said of detection of proteins using ligands such as antibodies). Probes can be labeled according to numerous well-known methods (Sambrook et al., 1989, supra). Non-limiting examples of detectable markers include ligands, fluorophores, chemiluminescent agents, enzymes, and antibodies. Other detectable markers for use with probes, which can enable an increase in sensitivity of the method of the invention, include biotin and radionucleotides. It will be understood by the person of ordinary skill that the choice of a particular label dictates the manner in which it is bound to the probe.

As commonly known, radioactive nucleotides can be incorporated into probes of the invention by several methods. Non-limiting examples thereof include kinasing the 5′ ends of the probes using gamma ³²P ATP and polynucleotide kinase, using the Klenow fragment of Pol I of E. coli in the presence of radioactive dNTP (e.g. uniformly labeled DNA probe using random oligonucleotide primers in low-melt gels), using the SP6/T7 system to transcribe a DNA segment in the presence of one or more radioactive NTP, and the like.

Amplification of a selected, or target, nucleic acid sequence may be carried out by a number of suitable methods. See generally Kwoh et al., 1990, Am. Biotechnol. Lab. 8:14-25. Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the Qβ replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra). Preferably, amplification will be carried out using PCR.

Polymerase chain reaction (PCR) is carried out in accordance with known techniques. See, e.g., U.S. Pat. Nos. 4,683,195; 4,683,202; 4,800,159; and 4,965,188 (the disclosures which are incorporated herein by reference). In general, PCR involves, a treatment of a nucleic acid sample (e.g., in the presence of a heat stable DNA polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analyzed to assess whether the sequence or sequences to be detected are present. Detection of the amplified sequence may be carried out by visualization following Ethidium Bromide (EtBr) staining of the DNA following gel electrophoresis, or using a detectable label in accordance with known techniques, and the like. For a review on PCR techniques (see PCR Protocols, A Guide to Methods and Amplifications, Michael et al. Eds, Acad. Press, 1990).

Ligase chain reaction (LCR) is carried out in accordance with known techniques (Weiss, 1991, Science 254:1292). Adaptation of the protocol to meet the desired needs can be carried out by a person of ordinary skill. Strand displacement amplification (SDA) is also carried out in accordance with known techniques or adaptations thereof to meet the particular needs (Walker et al., 1992, Proc. Natl. Acad. Sci. USA 89:392-396; and ibid., 1992, Nucleic Acids Res. 20:1691-1696).

“Nucleic acid hybridization” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 1989, supra and Ausubel, et al. (eds), 1989, Current Protocols in Molecular Biology, Vol. 1, Green Publishing Associates, Inc., and John Wiley & Sons, Inc., New York,) and are commonly known in the art. Hybridization to filter-bound sequences under moderately stringent conditions may, for example, be performed in 0.5 M NaHPO₄, 7% sodium dodecyl sulfate (SDS), 1 mM EDTA at 65° C., and washing in 0.2×SSC/0.1% SDS at 42° C. (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO₄, 7% SDS, 1 mM EDTA at 65° C., and washing in 0.1×SSC/0.1% SDS at 68° C. (see Ausubel, et al. (eds), 1989, supra). In other examples of hybridization, a nitrocellulose filter can be incubated overnight at 65° C. with a labeled probe in a solution containing 50% formamide, high salt (5×SSC or 5×SSPE), 5× Denhardt's solution, 1% SDS, and 100 μg/ml denatured carrier DNA (i.e. salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y.). The selected temperature is based on the melting temperature (Tm) of the DNA hybrid (Sambrook et al. 1989, supra). Generally, stringent conditions are selected to be about 5° C. lower than the thermal melting point for the specific sequence at a defined ionic strength and pH. Of course, RNA-DNA hybrids can also be formed and detected. In such cases, the conditions of hybridization and washing can be adapted according to well-known methods by the person of ordinary skill. Stringent conditions will be preferably used (Sambrook et al., 1989, supra).

The invention also provides an isolated, substantially pure, or recombinant altered NLRP7 polypeptide comprising one or more of the alterations defined above. The invention further provides an isolated nucleic acid encoding the above-mentioned altered NLRP7 polypeptide. The invention further provides an isolated altered NLRP7 nucleic acid comprising the above noted alteration. The invention further provides an isolated, substantially pure, or recombinant polypeptide encoded by the above-mentioned nucleic acid, as well as fusion proteins comprising the polypeptide and an additional polypeptide sequence (e.g., a heterologous polypeptide sequence). The invention further provides isolated nucleic acids having a nucleotide sequence which is substantially identical to the above-noted altered NLRP7 nucleic acid of the invention. The invention further provides an isolated, substantially pure, or recombinant polypeptide having an amino acid sequence which is substantially identical to the above-noted altered NLRP7 polypeptide of the invention.

“Homology” and “homologous” refers to sequence similarity between two peptides or two nucleic acid molecules. Homology can be determined by comparing each position in the aligned sequences. A degree of homology between nucleic acid or between amino acid sequences is a function of the number of identical or matching nucleotides or amino acids at positions shared by the sequences. As the term is used herein, a nucleic acid sequence is “homologous” to another sequence if the two sequences are “substantially identical”, as used herein, and the functional activity of the sequences is conserved (as used herein, the term ‘homologous’ does not infer evolutionary relatedness). Two nucleic acid sequences are considered “substantially identical” if, when optimally aligned (with gaps permitted), they share at least about 50% sequence similarity or identity, or if the sequences share defined functional motifs. In alternative embodiments, sequence similarity in optimally aligned substantially identical sequences may be at least 60%, 70%, 75%, 80%, 85%, 90% or 95%. As used herein, a given percentage of homology between sequences denotes the degree of sequence identity in optimally aligned sequences. The invention thus further provides a nucleic acid comprising a nucleotide sequence having at least 60%, 70%, 75%, 80%, 85%, 90% or 95% identity with an altered version of any of SEQ ID NOs 1 to 8 comprising an alteration noted herein or any combination of the alterations noted herein. An “unrelated” or “non-homologous” sequence shares less than 40% identity, though preferably less than about 25% identity, with any of the SEQ ID NOs described herein.

Substantially complementary nucleic acids are nucleic acids in which the complement of one molecule is “substantially identical” to the other molecule. Two nucleic acid or protein sequences are considered “substantially identical” if, when optimally aligned, they share at least about 70% sequence identity. In alternative embodiments, sequence identity may for example be at least 75%, at least 80%, at least 85%, at least 90%, or at least 95%. Optimal alignment of sequences for comparisons of identity may be conducted using a variety of algorithms, such as the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85: 2444, and the computerised implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al., 1990, J. Mol. Biol. 215:403-10 (using the published default settings). Software for performing BLAST analysis may be available through the National Center for Biotechnology Information (through the internet at http://www.ncbi.nlm.nih.gov/). The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighbourhood word score threshold. Initial neighbourhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program may use as defaults a word length (VV) of 11, the BLOSUM62 scoring matrix (Henikoff and Henikoff, 1992, Proc. Natl. Acad. Sci. USA 89: 10915-10919) alignments (B) of 50, expectation (E) of 10 (or 1 or 0.1 or 0.01 or 0.001 or 0.0001), M=5, N=4, and a comparison of both strands. One measure of the statistical similarity between two sequences using the BLAST algorithm is the smallest sum probability (P(N)), which provides an indication of the probability by which a match between two nucleotide or amino acid sequences would occur by chance. In alternative embodiments of the invention, nucleotide or amino acid sequences are considered substantially identical if the smallest sum probability in a comparison of the test sequences is less than about 1, preferably less than about 0.1, more preferably less than about 0.01, and most preferably less than about 0.001.

An alternative indication that two nucleic acid sequences are substantially complementary is that the two sequences hybridize to each other under moderately stringent, or preferably stringent, conditions. Examples of nucleic acid hybridization conditions are described above.

The invention further provides a vector comprising the above-mentioned altered NLRP7 nucleic acid. The term “vector” is commonly known in the art and defines a plasmid DNA, phage DNA, viral DNA and the like, which can serve as a DNA vehicle into which DNA of the present invention can be cloned. Numerous types of vectors exist and are well known in the art. In an embodiment, the above-mentioned vector is a recombinant vector.

In an embodiment, the above-mentioned vector is operably-linked to a transcriptional regulatory sequence (e.g., a promoter). A first nucleic acid sequence is “operably-linked” with a second nucleic acid sequence when the first nucleic acid sequence is placed in a functional relationship with the second nucleic acid sequence. For instance, a promoter is operably-linked to a coding sequence if the promoter affects the transcription or expression of the coding sequences. Generally, operably-linked DNA sequences are contiguous and, where necessary to join two protein coding regions, in reading frame. However, since for example enhancers generally function when separated from the promoters by several kilobases and intronic sequences may be of variable lengths, some polynucleotide elements may be operably-linked but not contiguous. “Transcriptional regulatory sequence/element” is a generic term that refers to DNA sequences, such as initiation and termination signals, enhancers, and promoters, splicing signals, polyadenylation signals which induce or control transcription of protein coding sequences with which they are operably-linked. “Promoter” refers to a DNA regulatory region capable of binding directly or indirectly to RNA polymerase in a cell and initiating transcription of a downstream (3′ direction) coding sequence. For purposes of the present invention, the promoter is bound at its 3′ terminus by the transcription initiation site and extends upstream (5′ direction) to include the minimum number of bases or elements necessary to initiate transcription at levels detectable above background. Within the promoter will be found a transcription initiation site (conveniently defined by mapping with S1 nuclease), as well as protein binding domains (consensus sequences) responsible for the binding of RNA polymerase. Eukaryotic promoters will often, but not always, contain “TATA” boxes and “CCAT” boxes. Prokaryotic promoters contain Shine-Dalgarno sequences in addition to the −10 and −35 consensus sequences.

The recombinant expression vector of the present invention can be constructed by standard techniques known to one of ordinary skill in the art and found, for example, in Sambrook et al. (supra). A variety of strategies are available for ligating fragments of DNA, the choice of which depends on the nature of the termini of the DNA fragments and can be readily determined by persons skilled in the art. The vectors of the present invention may also contain other sequence elements to facilitate vector propagation (e.g. a replicon) and selection in bacteria and host cells. In addition, the vectors of the present invention may comprise a sequence of nucleotides for one or more restriction endonuclease sites. Coding sequences such as for selectable markers and reporter genes are well known to persons skilled in the art.

A recombinant expression vector comprising a nucleic acid sequence of the present invention may be introduced into a host cell, which may include a living cell capable of expressing the protein coding region from the defined recombinant expression vector. The living cell may include both a cultured cell and a cell within a living organism. Accordingly, the invention also provides host cells containing the recombinant expression vectors of the invention. The terms “host cell” and “recombinant host cell” are used interchangeably herein. Such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

Vector DNA can be introduced into cells via conventional transformation or transfection techniques. The terms “transformation” and “transfection” refer to techniques for introducing foreign nucleic acid into a host cell, including calcium phosphate or calcium chloride co-precipitation, DEAE-dextran-mediated transfection, lipofection, electroporation, microinjection and viral-mediated transfection. Suitable methods for transforming or transfecting host cells can for example be found in Sambrook et al. (supra), and other laboratory manuals.

Recombinant production is useful for the preparation of large quantities of the protein encoded by the DNA sequence of interest. The protein can be purified according to standard protocols that take advantage of the intrinsic properties thereof, such as size and charge (e.g., SDS gel electrophoresis, gel filtration, centrifugation, ion exchange chromatography, etc.). In addition, the protein of interest can be purified via affinity chromatography using polyclonal or monoclonal antibodies or other affinity-based systems (e.g., using a suitable incorporated “tag” in the form of a fusion protein and its corresponding ligand). Suitable recombinant systems include prokaryotic and eukaryotic expression systems, which are known in the art.

The invention further provides an immunological reagent, such as an antibody, which exhibits different immunoreactivity with an altered NLRP7 polypeptide, i.e., comprising the above-noted alteration, relative to a wild-type NLRP7 polypeptide.

A further aspect of the invention provides an antibody that specifically recognizes an altered NLRP7 polypeptide of the invention. “Specifically recognizes” as used herein means that the antibody binds with a higher affinity to an altered NLRP7 polypeptide relative to other polypeptides, and more particularly to a “native” NLRP7 polypeptide that do not contain the alteration(s). Antibodies may be recombinant, e.g., chimeric (e.g., constituted by a variable region of murine origin associated with a human constant region), humanized (a human immunoglobulin constant backbone together with hypervariable region of animal, e.g., murine, origin), and/or single chain. Both polyclonal and monoclonal antibodies may also be in the form of antigen-binding immunoglobulin fragments, e.g., F(ab)′₂, Fab or Fab′ fragments. The antibodies of the invention are of any isotype, e.g., IgG or IgA, and polyclonal antibodies are of a single isotype or a mixture of isotypes. In general, techniques for preparing antibodies (including monoclonal antibodies and hybridomas) and for detecting antigens using antibodies are well known in the art.

Antibodies against the altered NLRP7 polypeptide of the present invention are generated by immunization of a mammal with a partially purified fraction comprising altered NLRP7 polypeptide (a polypeptide or a portion thereof containing the alteration(s)). Such antibodies may be polyclonal or monoclonal. Methods to produce polyclonal or monoclonal antibodies are well known in the art. For a review, see Harlow and Lane (1988) and Yelton et al. (1981), both of which are herein incorporated by reference. For monoclonal antibodies, see Kohler and Milstein (1975), and Campbell, 1984, In “Monoclonal Antibody Technology: Laboratory Techniques in Biochemistry and Molecular Biology”, Elsevier Science Publisher, Amsterdam, The Netherlands.

The antibodies of the invention, which are raised e.g., to a partially purified fraction comprising altered NLRP7 polypeptide of the invention, are produced and identified using standard immunological assays, e.g., Western blot analysis, dot blot assay, or ELISA. The antibodies are used in diagnostic methods to detect the presence of a altered NLRP7 polypeptide and activity in a sample, such as a tissue or body fluid. The antibodies are also used in affinity chromatography for obtaining a purified fraction comprising the altered NLRP7 polypeptide and activity of the invention.

The antibodies may be generated using the above-mentioned altered polypeptide, or a fragment thereof comprising one or more of the alterations described herein, as the antigen. Such antibody should be selected for preferential or specific binding to an altered NLRP7 polypeptide relative to a native NLRP7 polypeptide. For alterations resulting in a frameshift of the coding sequence, the region C-terminal to the alteration, which will comprises an amino acid sequence unrelated to the native NLRP7 due to the frameshift, may be used a an antigen to generate a specific antibody. For alterations resulting in a premature termination of the polypeptide (i.e., incorporation of a premature stop codon), the C-terminal end portion of the “premature” or “truncated” NLRP7 polypeptide, which is distinguishable for the corresponding portion in the native NLRP7 that contain the additional natural C-terminal residues, may be used as an antigen to generate a specific antibody. Such a strategy is typically used in the art to generate antibodies specific for a particular protease-generated fragment of a protein (i.e. to distinguish the fragment from the full-length protein), for example. For alterations resulting in a point mutation, a polypeptide or peptide containing the point mutation may be used for immunization, and antibodies showing preferential or specific binding to the mutated NLRP7 polypeptide/peptide relative to a native NLRP7 polypeptide/peptide not containing the point mutation are selected.

Accordingly, a further aspect of the invention provides (i) a reagent for detecting the presence of altered NLRP7 polypeptide and activity in a tissue or body fluid; and (ii) a diagnostic method for detecting the presence of altered NLRP7 polypeptide and activity in a tissue or body fluid, by contacting the tissue or body fluid with a reagent (e.g., an antibody of the invention or an antigen-binding fragment thereof) for detecting an altered NLRP7 polypeptide, such that a complex (e.g., an immune complex) is formed, and by detecting such complex to indicate the presence of altered NLRP7 polypeptide and activity in the sample or the organism from which the sample is derived. The detection of the altered NLRP7 polypeptide in the sample is an indication that the subject has a predisposition for recurrent reproductive wastage.

Those skilled in the art will readily understand that the immune complex is formed between a component of the sample and the antibody, and that any unbound material is removed prior to detecting the complex. It is understood that an antibody of the invention or an antigen-binding fragment thereof is used for screening a sample, such as, for example, blood, plasma, lymphocytes, cerebrospinal fluid, urine, saliva, epithelia and fibroblasts, for the presence of an altered NLRP7 polypeptide.

For diagnostic applications, the reagent (i.e., the antibody of the invention or an antigen-binding fragment thereof) is either in a free state or immobilized on a solid support, such as a tube, a bead, or any other conventional support used in the field. Immobilization is achieved using direct or indirect means. Direct means include passive adsorption (non-covalent binding) or covalent binding between the support and the reagent. By “indirect means” is meant that an anti-reagent compound that interacts with a reagent is first attached to the solid support. Indirect means may also employ a ligand-receptor system, for example, where a molecule such as a vitamin is grafted onto the reagent and the corresponding receptor immobilized on the solid phase. This is illustrated by the biotin-streptavidin system. Alternatively, a peptide tail is added chemically or by genetic engineering to the reagent and the grafted or fused product immobilized by passive adsorption or covalent linkage of the peptide tail.

In an embodiment, the immunological reagent (e.g., antibody or an antigen-binding fragment thereof) is conjugated to a moiety to facilitate the direct or indirect detection of the immune complex (i.e., antibody—altered NLRP7 complex). Such a moiety, may be for example, a ligand (e.g., biotin), a fluorophore, a chemiluminescent agent, an enzyme (e.g., horseradish peroxidase, green fluorescent protein, alkaline phosphatase), etc. In another embodiment, a second antibody recognizing the first antibody is used (indirect detection). Such second antibody may also be conjugated to a moiety (e.g., fluorophore, ligand, enzyme) to facilitate the direct or indirect detection of the immune complex (i.e., altered NLRP7-first antibody-second antibody complex).

The present invention also relates to a kit for diagnosing a condition of the female reproductive system, or a predisposition to having or developing same, comprising one or more suitable reagents to detect the above-mentioned alteration, such as a probe, primer (or primer pair), and/or an immunological reagent (e.g., antibody or an antigen-binding fragment thereof) in accordance with the present invention. For example, a compartmentalized kit in accordance with the present invention includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers may for example include a container which will accept the test sample (DNA, protein or cells), a container which contains the primers used in the assay, containers which contain enzymes, containers which contain wash reagents, and containers which contain the reagents used to perform the method and/or detect the indicator products (buffers, solutions, enzymes, etc.). In an embodiment the kit further comprises instructions for diagnosing a condition of the female reproductive system (e.g., reproductive wastage), or a predisposition to having or developing same.

Diagnostic (IVD)/Prognostic Tools

Presently, there are no available clinical tests to predict SAs and other types of pregnancy complications due to maternal defects. The identified set of mutations in the NRLP7 gene may be part of an in vitro diagnostic (IVD)/prognostic tool, which would enable patients to make more educated choices. Also, a test on a biological sample from the mother (e.g., maternal blood) would have the advantage not to require invasive sampling from the fetus. The following patients could benefit from such test:

(1) Patients who have had recurrent SAs or other types of RW and who would like to know their risk of recurrence and chances of having their own biological children from natural conceptions. Patients that are positive for NLRP7 mutations have high risk for recurrence and low chances of having children of their own, depending on the number and nature of mutations.

(2) Patients who are contemplating assisted reproductive technologies (ART). The advantage of testing this category of patients is tremendous, since detecting the type and number of mutations in the NLRP7 gene would enable a genetic counselor/clinician to counsel the patient prior to commencing this expensive and emotionally demanding treatment.

(3) For all patients, the knowledge of the number and type of mutations in the NLRP7 gene would enable the clinician to monitor the patient more closely and increase the chances of a viable pregnancy. It is well-known that proper monitoring of pregnancies with placental abnormalities, intrauterine growth retardation increases the survival of the fetus. A minority of patients would be expected to carry 2 NLRP7 defective alleles and only these patients are at high risk for recurrence and have low chances of having their own children (3% of their pregnancies). Such patients could benefit from preimplantation genetic diagnosis (PGD) as the transfer of diploid embryos to them would enhance their chances of conceiving their own children.

The present invention is illustrated in further details by the following non-limiting examples.

### EXAMPLES

### Example 1

### Materials and Methods

Patients. All patients and controls provided written consents to participate in this study. Patients were ascertained by (i) referral to us from various collaborators for genetic testing, (ii) recruitment from the miscarriage clinic of the McGill Reproductive Center, and (iii) referral from the Quebec and Montpellier registries of trophoblastic diseases. Selection criteria were either at least one HM (≧1 HM) or one trophoblastic disease or the occurrence of at least 3 spontaneous abortions (≧3 SAs). For most patients, clinical information was collected using standard pro forma recapitulating complete reproductive, medical, and family histories. One patient, 428, with one NLRP7 mutation previously reported by our group [16] had had in her last pregnancy a prematurely born baby at 28 weeks. The baby was later diagnosed with several congenital abnormalities including bilateral club foot, intraventricular hemorrhage grade II on the left side of the brain, developmental delay, mild tracheomalacia, patent ductus atresia that required several surgeries. Blood karyotype analysis, at a resolution level of 400 bands, revealed a 46,XY normal karyotype in 11 analyzed metaphases.

For mutation analysis, control DNA were from women either from the CEPH families or from women, of European descent, from families with various inherited conditions, unrelated to pregnancy losses, and with 5 to 16 children. However, their complete reproductive history and whether they had had reproductive wastage is not known.

Mutation analysis and annotation. Mutation analysis was performed as previously described [20] by PCR amplification of genomic DNA of the 11 NLRP7 exons followed by direct sequencing in the two directions. Sequences were analyzed using DNASTAR. In the text, we use the term mutations to indicate DNA changes, leading to protein truncations or NSVs that were not found in any of the tested controls including those of the same, or of related, ethnicities to the patients. Nucleotide numbering for mutations and variants uses cDNA numbering with +1 corresponding to the A of the ATG translation initiation codon in the reference sequence, NM_{—}001127255.1 (FIG. 8D, SEQ ID NO:8).

Cytokine secretion and western blotting. Peripheral blood mononuclear cells were separated using Ficoll™, counted, and cultured in the absence or the presence of ultrapure lipopolysaccharides (LPS) (1 μg/ml) (Cedarlane 423[LB] from E. coli 0552:B5) for 24 hours. Supernatants were collected and assayed by ELISA for MB and TNF secretion (BD Biosciences). Statistical analyses were done using ANOVA single factor analysis given it is the appropriate comparison test to deal with a single independent variable between all the compared values. P-value <0.05 was considered as statistically significant. Western blot analysis was performed using monoclonal antibodies directed against human IL1B (Cell Signaling technology, USA), p-IKB□ (Cell Signaling technology, USA), alpha-tubulin (Cell Signaling technology, USA) (1:1000) and beta-actin (Chemicon international). Protein bands were revealed using the Hyperfilm™ ECL Western blotting detection reagents (GE Healthcare, USA) and quantified by Image J software (http://rsb.info.nih.gov/ij/).

Histopathology. For histopathological diagnoses of CHM, PHM, and SA, a total of 105 tissue sections from 31 POCs were stained with haematoxilin and eosin, examined independently by two pathologists with large expertise in early pregnancies, scored for four parameters and classified as CHM, PHM, and SA. The four parameters are the presence of nucleated red blood cells inside chorionic villi, presence of fetal membranes or tissues beside chorionic villi, the degree of trophoblast proliferation, and the degree of hydropic changes. For late and term placentas tissues were screened independently by two other pathologists with extensive expertise in term placentas.

### Example 2

### NLRP7 Mutations in the Spectrum of Reproductive Wastage

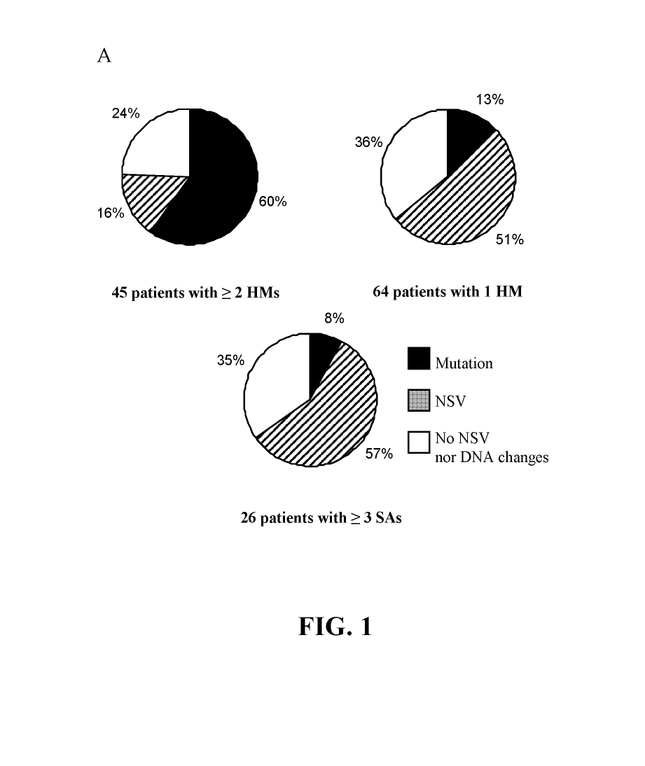

NLRP7 was sequenced in 135 unrelated patients with ≧1 HM or ≧3 SAs, of which 115 are new patients. Of the 135 unrelated patients 45 had had ≧2 HMs, 64 had had only 1 HM (with or without other reproductive wastage), and 26 had had ≧3 SAs (FIG. 1). The highest frequency of mutations was found in patients with ≧2 HMs, 60% (26 out of 45 patients), followed by patients with one HM, 13% (8 out of 64 patients), then patients with ≧3 SAs, 8% (2 out of 26 patients). Among the eight patients with 1 HM and NLRP7 mutations, six (75%) had had at least two additional reproductive wastage, one had had one mole and two live births, and the remaining had had one mole, but no data are available about her other reproductive outcomes. This indicates that NLRP7 mutations predispose patients for recurrent reproductive wastage rather than for sporadic moles. Among the 26 women with ≧3 SAs, two have NLRP7 mutations. One had had 2 live births and 7 SAs and is heterozygous for R156Q. The second patient had had four SAs, one of which led to a gestational trophoblastic disease that required methotrexate treatment, and is heterozygous for A719V. Both mutations, R156Q and A719V, were previously seen in women with moles [16] and were not found, respectively, on 310 and 200 chromosomes from control women of matching ethnicity to that of the two patients (both of European descent). This demonstrates that NLRP7 is also responsible for some cases of recurrent spontaneous abortions and that the variability in the reproductive outcomes of patients with the same mutation may be due to the genetic background of the patients, environmental factors, or both.

### Example 3

### Identification of Novel NLRP7 Mutations and Variants Including Three Protein-Truncating

Among 135 unrelated patients sequenced, 38 had at least one protein-truncating mutation or non synonymous variant that were not seen in controls of matching ethnicity. Among these mutations, there were a total of 30 different mutations, of which 20, were previously reported [15, 16, 17, 18, 19, 20, 21, 22] and ten are new. Four of the new mutations are protein-truncating, c.2616C>A, p.p.Tyr872X; c.930_{—}931del, p.Gln310HisfsX38; c.1018_{—}1020delinsCAAAA, p.Glu340GlnfsX11; c.2791_{—}2792delTG, p.Cys931X and the remaining six are missense, c.1976G>T, p.Arg659Leu; c.750C>A, p.Phe250Leu; c.1018G>A, p.Glu340Lys; c.1169G>A, p.Arg390His; c.1237C>T, p.Arg413Trp, and c.2444G>A, p.Arg815His. None of these six missense mutations was found in controls from several ethnic groups including those of the patients (Tables 1a and 1b). Two missense mutations, E340K and R413W, were predicted by Polyphen-2 to be probably damaging while the remaining, F250L, R390H, and R815H, were predicted to be more benign (Table 2). The reproductive outcomes of the patients with the new mutations and variants are shown in Table 3.

TABLES 1a and 1b
Number of screened control chromosomes from different ethnic
groups for the various mutations and variants

| | | Women of | | | |
|---|---|---|---|---|---|
| | | European | | | |
| | | descent | | | |
| | | with | Lebanese | | Pakistani |
| | Ethnic origin | 5 to 16 | (general | Chinese (general | (general |
| New mutation | of the patient | children | population) | population) | population) |
| c.750C > A, p.Phe250Leu | African/Indian | 208 | 42 | | 76 |
| c.930_931delGC, p.Q310HfsX38 | Caucasian | 208 | | 98 | — |
| c.1018_1020delGAGinsCAAAA, | Caucasian | 208 | | 98 | — |
| p.Glu340GlnfsX10 | | | | | |
| c.1018G > A, p.Glu340Lys | Pakistani | 208 | | 98 | 76 |
| c.2444G > A, p.Arg815His | Algerian | 220 | | — | — |
| c.2791_2792delTG, p.Cys931X | Caucasian | 208 | | 94 | 76 |
| c.1169G > A, p.Arg390His | Guadeloupean | 208 | | 98 | 76 |
| c.1237C > T, p.Arg413Trp | Haitian | 208 | | 98 | 76 |
| c.1976G > T, p.Arg659Leu | Chinese | 338 | | 100 | 76 |
| c.2616C > A, p.p.Tyr872X | Mexican | 214 | | | |

| | | African American | |
|---|---|---|---|
| New mutation | Ethnic origin of the patient | (general population) | Total number of controls |
| c.750C > A, p.Phe250Leu | African/Indian | | 326 |
| c.930_931delGC, p.Q310HfsX38 | Caucasian | — | 306 |
| c.1018_1020delGAGinsCAAAA, | Caucasian | — | 688 |
| p.Glu340GlnfsX10 | | | |
| c.1018G > A, p.Glu340Lys | Pakistani | — | 382 |
| c.2444G > A, p.Arg815His | Algerian | — | 418 |
| c.2791_2792delTG, p.Cys931X | Caucasian | — | 378 |
| c.1169G > A, p.Arg390His | Guadeloupean | | 556 |
| c.1237C > T, p.Arg413Trp | Haitian | | 556 |
| c.1976G > T, p.Arg659Leu | Chinese | | 514 |
| c.2616C > A, p.p.Tyr872X | Mexican | | 214 |

TABLE 2
Correlation between predicted effects of missense mutations and variants
and reproductive outcomes of the patients

| | Polyphen-2 | The patients of the present study | | | General |
|---|---|---|---|---|---|
| Substitution | score | Reproductive wastage | | Live birth | population |
| C399Y | 0.999 | 1 | HM | yes^{#} | no |
| P716A | 0.997 | ≧2 | HMs | no | no |
| N913S | 0.994 | ≧2 | HMs | exceptionally^{$} | no |
| G380R | 0.987 | 1 | HM | no | no |
| L398R | 0.968 | ≧2 | HMs | no | no |
| L964P | 0.968 | ≧2 | HMs | no | no |
| E340K | 0.962 | 1 | HM | no | no |
| R693W | 0.959 | ≧2 | HMs | no | no |
| D657V | 0.951 | ≧2 | HMs | no | no |
| A719V | 0.942 | 1 HM or ≧3 SAs | | no | no |
| R413W | 0.915 | 1 | HM | yes | no |
| C84Y | 0.909 | 1 | HM | no | no |
| R693P | 0.906 | ≧2 | HMs | exceptionally^{$} | no |
| K277Q* | 0.843 | ≧2 | HMs | no | no |
| M192L* | 0.701 | ≧2 | HMs | no | yes |
| D722G | 0.677 | ≧2 | HMs | no | no |
| K379N | 0.663 | ≧2 | HMs | no | no |
| T1028A | 0.478 | ≧2 HMs or 1 HM | | yes | yes |
| R701C | 0.419 | ≧2 | HMs | no | no |
| F250L | 0.280 | 1 | HM | yes | yes |
| R693Q | 0.273 | ≧2 | HMs | no | no |
| M427T | 0.189 | 1 | HM | yes | yes |
| L750V | 0.110 | ≧2 | HMs | no | no |
| R156Q | 0.103 | ≧2 HMs or ≧3SAs | | yes | no |
| K511R | 0.087 | ≧3 | SAs | yes | yes |
| R390H | 0.0.041 | 1 | HM | yes | no |
| F430L | 0.033 | 1 | HM | yes | yes |
| L311I | 0.025 | 1 HM or ≧3 SAs | | yes | yes |
| Q310R | 0.007 | 1 HM or ≧3 SAs | | yes | yes |
| A481T | 0.007 | ≧2 HMs or 1 HM or ≧3 SAs | | yes | yes |
| V319I | 0.005 | ≧2 HMs or 1 HM or ≧3 SAs | | yes | yes |
| V699I | 0.003 | 1 | HM | n.a. | yes |
| G487E | 0.002 | 1 HM or ≧3 SAs | | yes | yes |
| R815H | 0.001 | 1 | HM | n.a. | no |

Polyphen-2 scores for human variations are listed by decreasing severity from top to bottom.
n.a., indicates no available data about the other reproductive outcomes of the patient.
Different outcomes in different patients are indicated by “or”.
*indicates missense variants found in patients on haplotypes carrying other mutations;
^{#}indicates the presence of several congenital malformations in the live birth and are described in the Materials and Methods' section;
^{$}indicates a single live birth in one patient who is compound heterozygous for N913S and R693P among 6 patients with N913S (who had had a total of 24 pregnancies) and 10 patients with R693P (who had had a total of 36 pregnancies).

TABLE 3
New mutations and reproductive outcomes of the patients

| Family ID | Patient ID | NLRP7 | Reproductive Outcomes of the patients |
|---|---|---|---|
| MoUs167 | 712 | p.[P716A]; [Cys931X] | SA, PHM, PHM |
| MoNz170 | 725 | p.[Q310HfsX38; A481T]; [R693W] | CHM, SA, CHM, HM |
| MoCa179 | 744 | p.[Glu340GlnfsX10]; [R693W] | HM, BO, 3 SA, 2 HM |
| MoCa186 | 758 | p.[V319I; G487E]; [V319I; G487E]; [F250L(;)A481T] | LB, CHM, LB |
| MoPa214 | 814 | p.[V319I(;)E340K] | SA, SA, PHM (uterus retroverted, small leiomyoma) |
| MoGu248 | 897 | p.[A481T]; [A481T]; [V319I(;)R390H(;)G487E] | ET, ET, SA, CHM, SA, SA, SA-CC, NP |
| MoHa259 | 919 | p.[V319I(;)R413W(;)G487E] | NP, PHM-GTN (I-5) |
| MoCh329 | 1036 | p.R659L; p.K379N | EA, ART(ICSI)-SA, ART (ICSI)-SA, ART(PGS)-SA |
| MoMx341 | 1074 | c.2810 + 2T > G = T/G; p.Tyr872X | SA-IM-CC, HM, 4 SA, 2 HM |

LB, stands for live birth;
HM, for hydatidiform mole;
PHM, partial HM;
CHM, complete HM;
SA, spontaneous abortion;
BO, blighted ovum;
ET, elective termination;
CC, choriocarcinoma;
GTN, gestational trophoblastic neoplasia grade I-5 according to HUPO nomenclature,
EA for elective abortion,
ART for assisted reproductive technologies,
ICSI, for intracytoplasmic sperm injection,
PGS for pre-implantation genetic screening,
IM for invasive mole.
“—” indicates that the concerned pregnancy lead to, for instance,
SA-IM-CC, indicates that the SA lead to an invasive mole and then to CC.
New mutations are in bold.

### Example 4

### Genotype-Phenotype Relationships of Mutations in NLRP7

Patients with one NLRP7 defective allele have better reproductive outcomes. Among the 36 unrelated patients with mutations from the three groups (≧2 HMs, 1 HM, or ≧3 SAs), 24 had two defective alleles and 12 had a single defective allele. Comparing the reproductive outcomes of patients with one and two defective alleles (including related patients in familial cases) revealed that patients with one defective allele had significantly more live births (18.4% versus 2.5%), more SAs (37% versus 17.4), and less HMs (34.1% versus 73.5% of their pregnancies) than patients with two defective alleles (Fisher exact test, p-value=2.809e-06) (FIG. 2A). Among the 12 patients, each with one defective allele, seven had had a single mole with other forms of reproductive wastage. These data indicate that patients with one identified defective allele most likely have a single defective allele and consequently better reproductive outcomes than individuals with 2 mutated alleles who have a much greater chance of having recurrent molar pregnancies.

Protein-truncating mutations are associated with repeat CHMs. To investigate possible correlations between the nature of the mutations and the histopathological types of the molar tissues, a total of 105 tissue sections from 31 POCs from 13 patients (from 12 unrelated families) with NLRP7 mutations were examined, scored, and diagnosed independently by two pathologists. Both pathologists noted that recurrent molar tissues from patients with NLRP7 mutations have, in general, less trophoblastic proliferation than common sporadic moles. There was an agreement between the diagnoses of the two pathologists in 80% of the cases. Among 10 HMs from patients with at least one protein-truncating mutation, 9 were diagnosed as CHMs by the two pathologists (FIG. 2B). Patients with missense mutations had more variability in the histopathological diagnosis of their POCs (FIG. 2B). These data demonstrate that repeat CHMs is the most severe phenotype caused by NLRP7.

In view of the association between CHMs and protein-truncating mutations, the frequencies of protein truncating mutations in all reported familial and singleton cases of recurrent moles was compared, with the hypothesis that if protein truncating mutations were associated with the severe phenotype, they should be more frequent in familial than in singleton cases since their presence would have favored the manifestation of moles in all family members carrying them. A recapitulation of these cases showed a higher frequency of protein-truncating mutations in familial than in singleton cases, 52.17% versus 32.71% (Table 4). Although, this association is not statistically significant, it indicates that some missense mutations are less penetrant and consequently not all family members carrying them manifest moles.

TABLE 4
Protein-truncating mutations in familial versus singleton cases

| | Familial cases | | Singletons | |
|---|---|---|---|---|
| | | ≧1 | | ≧1 |
| | N. of | Protein- | N. of | Protein- |
| References | Families | truncating | singleton | truncating |
| Murdoch et al. 2006 | 4 | 2 | 1 | 0 |
| Qian et al., 2007 | 1 | 1 | | |
| Kou et al., 2008 | 3 | 2 | 5 | 3 |
| Pueshberty et al., 2008 | | | 1 | 0 |
| Deveault et al., 2009 | 3 | 1 | 8 | 0 |
| Hayward et al., 2009 | 5 | 3 | 8 | 2 |
| Wang et al., 2009 | 7 | 3 | 13 | 5 |
| Current study | | | 5 | 3 |
| Total | 23 | 12 | 41 | 13 |

| | | |
|---|---|---|
| Protein-truncating | 52.17% | 31.71% |

N, stands for number.

Distribution of mutations and variants in the three NLRP7 domains. The distribution of the different mutations and variants found in the three NLRP7 domains is shown in FIGS. 2C and 2D. In this cohort of patients, protein truncating mutations were only found in patients with at least 2 HMs. Patients with only 1 HM or at least 3 spontaneous abortions had only missense variants (FIG. 2D). Other NLRP7 mutations and variants have been reported previously, and are listed on INFEVERS (INFEVERS: an online database for autoinflammatory mutations. Copyright. Available at http://fmf.igh.cnrs.fr/ISSAID/infevers/; references [26 to 28 and 30]) (FIG. 2D). The highest number of missense mutations (62%) is observed in the LRR which represent only 36% of the total size of the protein. Only 1 NSV, V699I, in the LRR has been seen among all controls analyzed from several ethnic groups as compared to 12 in the NACHT domain (319 aa, spanning residues 172 to 491 of NLRP7), which is even 15% shorter than the LRR domain (369 aa) (FIG. 2D).

The predicted functional consequences of all missense mutations by Polyphen-2 (reference [29], http://genetics.bwh.harvard.edu/pph2/) showed the association of missense variants with mild predicted functional consequences with the category of 1 HM or ≧3 SAs while severe mutations are associated with the occurrence of ≧2 HMs (Table 2).

### Example 5

### Rare Non-Synonymous Variants in NLRP7 are Associated with Recurrent Hydatidiform Moles and Spontaneous Abortions

To investigate whether the non-synonymous NLRP7 variants predispose women to reproductive wastage, women with at least one mutation and those of non-European origin were removed from the cohort of 135 women described above, and the frequencies of NSVs in 53 European patients with no mutations in NLRP7 was compared with 155 controls of European descent (Table 5).

TABLE 5
Frequencies of non-synonymous NLRP7 variants in patients and controls of European descent

| | | | | | | | ≧1 HM and | | |
|---|---|---|---|---|---|---|---|---|---|
| | | | | ≧1 HM or ≧3 SA | | | another RW or ≧3 SA | | |
| Variant/Mutation | | Minor | Controls | Patients | | | Patients | | |
| cDNA | Protein | alleles | (n = 105-155) | (n = 53) | Chi² | p-values | (n = 40) | Chi² | p-values |
| c.929A > G | Q310R* | R | 0.006 | 0.018 | 1.261 | | 0.024 | 2.043 | |
| c.931C > A | L311I* | I | 0.009 | 0.018 | 0.551 | | 0.024 | 1.098 | |
| c.955G > A | V319I | I | 0.185 | 0.169 | 0.725 | | 0.183 | 0.002 | |
| c.1280T > C | M427T* | T | 0.009 | 0.009 | 0.987 | | 0.012 | 0.038 | |
| c.1288T > C | F430L* | L | 0.004 | 0.009 | 0.237 | | 0.012 | 0.469 | |
| c.1441G > A | A481T* | T | 0.064 | 0.132 | 6.24 | 0.012 | 0.159 | 7.435 | 0.0063 |
| c.1460G > A | G487E* | E | 0.035 | 0.056 | 0.899 | | 0.061 | 1.076 | |
| c.1532A > G | K511R* | R | 0.018 | 0.028 | 0.338 | | 0.037 | 0.904 | |
| | Any of the above | | 0.479 | 0.66 | 4.52 | 0.033 | 0.750 | 8.401 | 0.0037 |
| | Any rare NSV | | 0.177 | 0.471 | 13.018 | 0.0003 | 0.550 | 19.198 | 0.000012 |

n, indicates the number of subjects in each category.
RW, indicates a reproductive wastage.
A total of 155 controls were analysed for all variants, except for M427T, F430L, and K511R, for which 105 controls were analyzed.
MAF, indicates minor allele frequency. Two by two contingency table was used for MAF higher than five in patients or controls, and Fisher exact test for values equal or lower than 5 (http://www.quantitativeskills.com/sisa/distributions/binomial.htm). Only significant p-values are indicated. Rare NSV indicates those with MAF ≦0.064 and are indicated by asterisks.

This analysis revealed a statistically significant association between c.1441G>A, p.A481T and reproductive wastage (Chi²=6.24, p-value=0.012). Among the 53 patients, 12 had had a single HM with either no data about their other reproductive outcomes or with normal pregnancies. When these cases were removed from the sample and only cases with one mole and at least another reproductive wastage or patients with ≧3SAs were included, the association with A481T was more significant (Chi²=7.435, p-value=0.0063). Five other rare NSVs were more frequent in the patients than in controls, but did not reach individually statistical significance (Table 5). The association between the presence of any of the NSVs or any of the rare NSVs listed in Table 5 and reproductive wastage was assessed, and a significant association was found with patients with 1 HM or ≧3SAs (p-value=0.0003) that was even higher after removing cases with 1 HM and no other reproductive wastage (p-value=0.000012). Altogether, the data support the role of A481T and the other rare NSVs in the genetic susceptibility for recurrent reproductive wastage.

### Example 6

### Low IL1B and TNF Secretion by Mononuclear Blood Cells from Patients with A481T

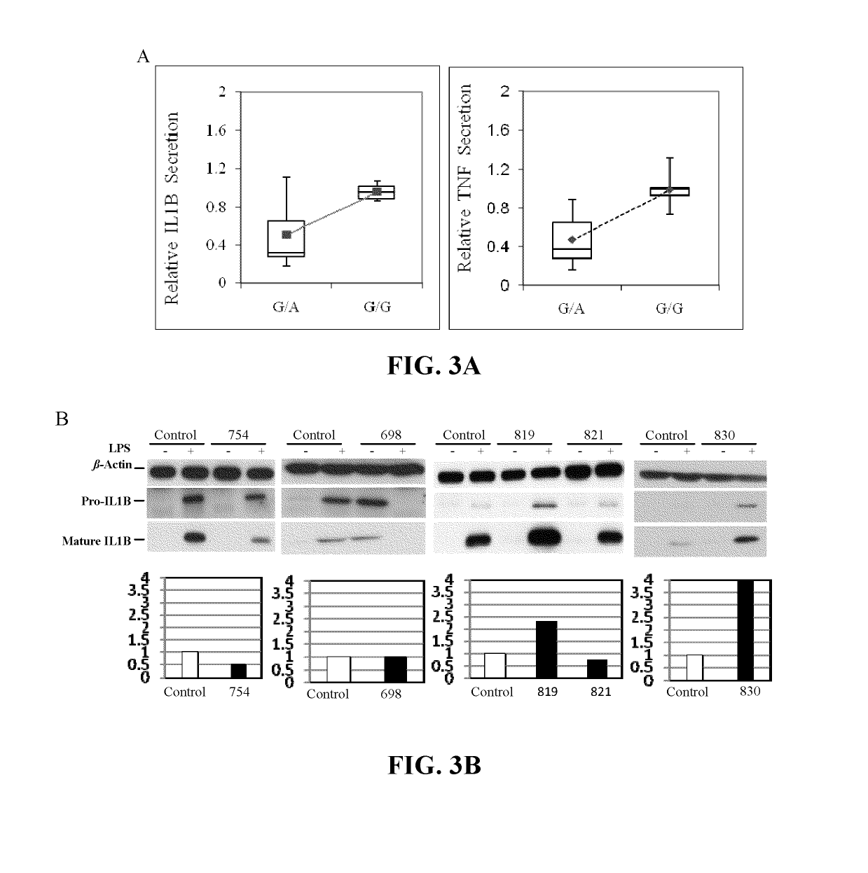

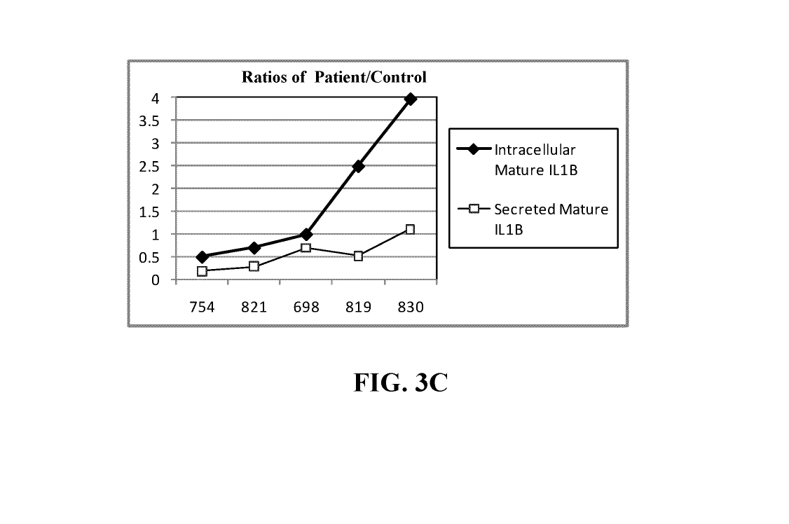

To investigate the potential functional consequences of A481T and the other rare NSVs on IL1B and TNF secretion, PBMCs from 5 patients, 698 (p.[V319I;A481T;G487E];[=]), 754 (p.[A481T];[=]), 819 (p.[A481T];[=]), 821 (p.[Q310R(;)L311I(;)A481T]), 830 (p.[A481T];[=]), carrying A481T with and without other NSVs and 7 different controls without A481T and any of the other rare NSVs were stimulated ex vivo and the levels of cytokines were determined by ELISA. This analysis demonstrated that patients' cells secrete statistically lower levels of IL1B and TNF as compared to cells from control subjects (p-value<0.001) (FIG. 3A). The levels of intracellular production of pro and mature IL1B by cells from these 5 patients and one control was determined using western blot analysis. Variable levels of intracellular pro and mature IL1B in different patients were observed (FIG. 3B) similar to those reported in healthy subjects [25]. In all the analyzed patients, the intracellular levels of mature IL1B mirrored those of pro-IL1B demonstrating that NSVs in NLRP7 do not affect IL1B cleavage. The ratios of intra and extracellular IL1B between cells from each of the five patients and the same control cultured, stimulated, and assayed at the same time was assessed. As shown in FIG. 3C, the ratios of secreted IL1B by patient cells relative to control cells (patient/control) are lower than the ratios of their intracellular mature ILIB, demonstrating that A481T and the other rare NSVs have functional consequences and reduce cytokine secretion upon stimulation with LPS.

Altogether, these data demonstrate the association of A481T and rare NSVs with lower cytokine secretion and lower NF-κB activation, similar to those observed in patients with NLRP7 mutations, and support further the role of these rare NSVs in conferring genetic susceptibility for reproductive wastage.

### Example 7

### Increased Perinatal Morbidities and Placental Abnormalities in Patients with NLRP7 Mutations or Rare NSVs

To date, six of the 46 patients studied with at least one NLRP7 mutation (13% of the patients) had had 7 stillbirths (3.4% of their pregnancies). Tissues from all the placentas of these stillborn babies were not available for evaluation. The descriptions provided by the patients for four cases indicated the death of morphologically normal babies (Table 6). Medical reports were available for two cases (Table 7). In one case, the patient manifested, at 26 weeks of gestation, preeclampsia, placental abruption. Infarction and calcification were diagnosed by histophathological examination of the placenta of the delivered baby who died later. In the second case, a placental haematoma was diagnosed by ultrasonography after the intrauterine demise of the baby.

TABLE 6
Description provided by the gynecologists about 4 stillbirths from patients
with NLRP7 mutations

| Family | Patient | | |
|---|---|---|---|
| ID | ID | Mutations | Description by the patient and available medical record |
| MoLb1 | 4 | p.[G118X]; [G118X] | According to the patient: GA 28 w, no preeclampsia, no bleeding. |
| | | p.[G118X]; [G118X] | According to the patient: GA full term pregnancy, home delivery |
| | | | of a baby 3.5 kb after uncomplicated pregnancy. |
| | | | The baby died one week later after suddening turning blue |
| | | | and being hypoxic for no apparent reason. |
| MoIn68 | 474 | p.[R693P]; [R693P] | According to the patient: GA full term pregnancy, vaginal delivery |
| | | | of a dead baby with no obvious mophological malformations. |
| | | | No preeclampsia during the pregnancy. |
| MoIn109 | 691 | p.[N913S]; [N913S] | According to the patient: GA 32 w. |

GA indicates gestational age;
w, indicates weeks

TABLE 7
Available medical information from 2 stillbirths from patients with NLRP7
mutations

| Family | | | |
|---|---|---|---|
| ID | Patient ID | Mutations | Description by the patient and available medical record |
| MoLb1 | 6 | p.[G118X]; [G118X] | Medical record: GA 26 w. Preterm labor and vaginal bleeding. The |
| | | | patient had preeclampsia with severe placental abruption. She then |
| | | | delivered a live male of 450 g (small for 26 w) who died later. |
| | | | Histopathology of the palcenta revealed normal decidua and villi |
| | | | with areas of infarction and calcification. |
| MoBa169 | 723 | p.[G380R]; [=] | Medical record: GA 29 w. Clinical manifestation: lower abdominal |
| | | | pain, reduced foetal movement for 7 days. Ultrasonography: Intra |
| | | | uterine fetal demise, no amniotic fluid was seen. There was a |
| | | | posterior haematoma in the placenta. Vaginal delivery of a |
| | | | morphologically normal dead fetus. |

In an attempt to understand what could have caused the death of these babies, placental tissues from one stillbirth and 8 live births from 6 patients with NLRP7 mutations or with rare NSVs, all of whom are living in Canada, were retrieved. Histopathological evaluation of the placentas by two pathologists with extensive expertise in term placentas revealed a number of abnormalities (Table 8). This evaluation showed that only 2 of the 9 analyzed placentas did not have any abnormality and 6 had one to several abnormalities with variable seventies. These abnormalities included mild to severe chorioamnionitis, inflammation of the chorion and amnion that was seen in 3 placentas (FIG. 9); mild chorangiosis, a placental sign associated with prolonged hypoxia that was seen in 3 placentas; decidual necrosis that was seen in 3 placentas; and dysmature stem chorionic villi, seen in 2 placentas (FIG. 9). The abnormalities seem to correlate with the severity of the allele, with severe abnormalities in patient 428 who has a mutation and milder abnormalities in patients with rare NSVs.

TABLE 8
Summary of histopathological evaluations of the placentas of patients with
NLRP7 mutations and at least one rare NSVs

| | | | | Clinical information & | | |
|---|---|---|---|---|---|---|
| Case ID | Patient ID | Mutation or Variant | N | Gross Morphology | Pathologist 1 | Pathologist 2 |
| MoCa57 | 428 | p.[V319I; G487E]; [V319I; C399Y] | 2 | GA 22 w. SB of a | Immature placenta, mild | Chorionitis, |
| | | | | morphologically normal | chorioamnionitis, decidual | subchorionic |
| | | | | male fetus, baby weight | necrosis. | hemorraghe |
| | | | | 465 g, 27 cm length, | | |
| | | | | placenta 10 × 10 cm, 163 | | |
| | | | | grams, cord inserted | | |
| | | | | centrally and mesures 11 cm. | | |
| | | | | No histopathology | | |
| | | | | done on the baby. The | | |
| | | | | patient had vaginal | | |
| | | | | spotting since 10 w, high | | |
| | | | | βHCG 160, 625 U/L, | | |
| | | | | vaginal infection, | | |
| | | | | antibiotics treatment, | | |
| | | | | bleeding again around 21 w, | | |
| | | | | lower abdominal pain | | |
| | | | | few days before the | | |
| | | | | stillbirth and was | | |
| | | | | admitted to the hospital. | | |
| | | | | Placenta removed | | |
| | | | | manually. | | |
| | | | 4 | GA 28 1/7 w, male live | Immature placenta, marked | Advanced villous |
| | | | | birth. True knot. Cervical | chorioamnionitis, surface | maturation, acute |
| | | | | incompetence, complete | vasculitis, funisitis, | vasculitis and |
| | | | | microbiology workout | deciduitis; mural | funisitis. Severe |
| | | | | negative, cerclage, bed | thrombosis of surface | acute |
| | | | | rest, and antibiotic | vessels. | chorioamnionitis |
| | | | | treatment. Preterm labor | | and deciduitis |
| | | | | and rupture of | | |
| | | | | membranes. | | |
| | | | | Chorioamnionitis. | | |
| | | | | Cesarean section. | | |
| | | | | Accessory lobe. The baby | | |
| | | | | was later diagnosed with | | |
| | | | | several congenital | | |
| | | | | malformations described | | |
| | | | | in Patients and Methods. | | |
| MoCa209 | 804 | p.[V319I(;) R156Q] | 2 | GA 40 6/7 w, 3.3 kg | Normal | Normal |
| | | | | female, 615 g, 18 × 14 × 2.5, | | |
| | | | | cord 30 cm, 3 vessels | | |
| | | | 2 | GA 39 3/7 w, 530 g, | Normal | Normal |
| | | | | 17 × 16 × 3 cm, cord 44 cm, | | |
| | | | | 3 vessels, false knot | | |
| MoCa182 | 754 | p.[A481T]; [=] | 1 | Live birth of a girl | Normal placenta with | Dysmature stem |
| | | | | | minimal necrosis of | villi and mild |
| | | | | | decidua. | chorangiosis |
| MoCa207 | 802 | p.[G487E]; [=] | 2 | GA 41 W, 824 g | Chorangiomas in the | Prominent |
| | | | | | surface near the insertion | perivillous fibrin |
| | | | | | of the cord | agregates, |
| | | | | | | chorangiosis |
| MoCa210 | 806 | p.[V319I(;) A481T] | 1 | GA 35 1/7 w, | Normal | Chorangiotic |
| | | | | spontaneous vaginal | | changes identified |
| | | | | delivery of a male baby, | | |
| | | | | 720 g, 21 × 18.5 × 2.8, 76 cm, | | |
| | | | | 1.4 cm, cord twisted, | | |
| | | | | one knot, 3 vessels | | |
| | | | | eccentrically | | |
| MoCa186 | 758 | p.[V319I; G487E]; [V319I; G487E]; | 3 | GA 40 5/7 w, birth weight | Very minimal | Chorangiotic |
| | | [F250L(;)A481T] | | 3670 g, female, | chorangiosis and focal | changes identified, |
| | | | | spontaneous vaginal | decidual necrosis. | subamniotic cyst |
| | | | | delivery, placenta 20 × 20, | | |
| | | | | cord 50 cm with 3 vessels, | | |
| | | | | central insertion | | |
| | | | 2 | GA 38 2/7 w, birth weight | Minimal chorangiosis and | Oedematous and |
| | | | | 4045 g, placenta weight | mild chorioamnionitis. | dysmature stem |
| | | | | 650 g, cord 53 cm and 1.2 cm, | | villi, chorangiosis |
| | | | | 3 vessels | | |

To investigate whether the abnormalities observed in the placentas from of the stillborn and the malformed male babies of patient, 428, with a mutation in NLRP7, are caused by placental mosaicism and the presence of androgenetic cells with two X chromosomes, fluorescent in situ hybridization were preformed using probes for the Y and the X chromosomes. This analysis did not reveal any XX cells in five available sections from the two placentas and all cells from the two placentas had normal numbers of sex chromosomes with one X and one Y. The presence of other chromosomal aneuploidies was tested with probes from four autosomes. Conclusive results were obtained on the placenta of the stillbirth with one probe from chromosome 18 and revealed 2 copies. More probes were conclusive on the placenta from the malformed baby and again revealed two copies of chromosomes 13, 18, 21, and 22. Therefore, mosaicism or aneuploidies was not detected in the available tissues from the two placentas of patient 428.

### Example 8

### NLRP7 Mutations Screening in Assisted Reproductive Technologies (ART)

The reproductive outcomes of 6 patients having NLRP7 mutations undergoing ART. None of the patients (3) with two mutated alleles conceived while 2 patients with one mutated allele each conceived and their babies are normal. These data are in agreement with the better reproductive outcomes of patients with one NLRP7 mutations from natural conceptions, as compared to patients with mutations in two alleles (Example 4). Thus, patients with two mutated allele could benefit from ovum donation. It was also noted that patients with mutations in the NACHT domain have higher rates of postzygotic aneuploidies and mosaicisms than the average observed in women undergoing ART meaning that these patients would benefit from preimplantation genetic diagnosis (PGD) for aneuploidies to transfer to them diploid embryos and increase their chances of having normal pregnancies.

### Example 9

### Summary of Key Involvements of NLRP7 Mutations in the Spectrum of Reproductive Wastage

1) Protein-truncating mutations are associated with repeated complete hydatidiform moles (CHMs). Patients with at least one protein-truncating mutation have mostly complete moles while those with missense mutations had more variability in the histopathological diagnosis of their products of conception (POCs). These data demonstrated that repeat CHMs are the most severe phenotype caused by NLRP7.

2) Patients with one NLRP7 defective allele have better reproductive outcomes. It was found that one defective allele is associated with more live births, more SAs, and fewer HMs. These correlations indicate that NLRP7 testing can be used to predict reproductive outcomes (a predisposition to recurrent reproductive wastage) of the patients and provide appropriate genetic counseling.

3) Overlap between HMs and SAs. Patients with NLRP7 mutations do not have only RHMs but also all the other forms of reproductive wastage (RW). Besides the moles, the most common form of RW in these patients is SAs and account for 19.5% of their pregnancies. The highest frequency of NLRP7 mutations was found in patients with at least 2 HMs followed by patients with only 1 HM and then by patients with at least 3 SAs and no moles.

4) Association between NSVs in NLRP7 and recurrent reproductive wastage. By comparing the frequencies of non-synonymous variants (NSVs) in European patients and controls, it was demonstrated that NLRP7 NSVs predispose the patients to RW. For example, A481T was statistically more frequent in patients than in controls. The data support the role of A481T and the other NSVs in the genetic susceptibility for recurrent RW23.

5) Increased perinatal morbidities and placental abnormalities in patients with NLRP7 mutations or rare NSVs. Six of of 46 patients with at least one NLRP7 mutation (13%) had had stillbirths (3.4% of their pregnancies). In a first attempt to understand what could have caused the death of these babies, placental tissues were retrieved from one stillbirth and 8 live births from 6 patients with NLRP7 mutations or with rare NSVs, all of whom are living in Canada. Histopathological evaluation of the placentas revealed that only 2 of the 9 analyzed placentas did not have any abnormality and 6 had one to several abnormalities with variable severities.

6) NLRP7 and assisted reproductive technologies (ART). Patients undergoing ART and having two mutated NLRP7 alleles are less likely to conceive as compared to patients with one mutated allele. Also, patients with mutations in the NACHT domain have higher rates of postzygotic aneuploidies and mosaicisms than the average observed in women undergoing ART meaning that these patients would benefit from preimplantation genetic diagnosis (PGD) for aneuploidies to transfer to them diploid embryos and increase their chances of having normal pregnancies.

Although the present invention has been described hereinabove by way of specific embodiments thereof, it can be modified, without departing from the spirit and nature of the subject invention as defined in the appended claims. Throughout this application, various references are referred to describe more fully the state of the art to which this invention pertains. The disclosures of these references are hereby incorporated by reference into the present disclosure.

### REFERENCES

1 Seckl M J, Sebire N J, Berkowitz R S. Gestational trophoblastic disease. Lancet 2010.

2 Berkowitz R S, Im S S, Bernstein M R, Goldstein D P. Gestational trophoblastic disease. Subsequent pregnancy outcome, including repeat molar pregnancy. J Reprod Med 1998;43(1):81-6.

3 Horn L C, Kowalzik J, Bilek K, Richter C E, Einenkel J. Clinicopathologic characteristics and subsequent pregnancy outcome in 139 complete hydatidiform moles. Eur J Obstet Gynecol Reprod Biol 2006;128(1-2):10-4.

4 Kim J H, Park D C, Bae S N, Namkoong S E, Kim S J. Subsequent reproductive experience after treatment for gestational trophoblastic disease. Gynecol Oncol 1998;71(1):108-12.

5 Kronfol N M, Iliya F A, Hajj S N. Recurrent hydatidiform mole: a report of five cases with review of the literature. J Med Liban 1969;22(4):507-20.

6 Sand P K, Lurain J R, Brewer J I. Repeat gestational trophoblastic disease. Obstet Gynecol 1984;63(2):140-4.

7 Sebire N J, Fisher R A, Foskett M, Rees H, Seckl M J, Newlands E S. Risk of recurrent hydatidiform mole and subsequent pregnancy outcome following complete or partial hydatidiform molar pregnancy. BJOG: An International Journal of Obstetrics and Gynaecology 2003;110(1):22-6.

8 Lan Z, Hongzhao S, Xiuyu Y, Yang X. Pregnancy outcomes of patients who conceived within 1 year after chemotherapy for gestational trophoblastic tumor: a clinical report of 22 patients. Gynecol Oncol 2001;83(1):146-8.

9 Van Thiel D H, Ross G T, Lipsett M B. Pregnancies after chemotherapy of trophoblastic neoplasms. Science 1970;169(952):1326-7.

10 Szulman A E, Surti U. The syndromes of hydatidiform mole. II. Morphologic evolution of the complete and partial mole. Am J Obstet Gynecol 1978;132(1):20-7.

11 Fukunaga M, Katabuchi H, Nagasaka T, Mikami Y, Minamiguchi S, Lage J M. Interobserver and intraobserver variability in the diagnosis of hydatidiform mole. Am J Surg Pathol 2005;29(7):942-7.

12 Acaia B, Parazzini F, La Vecchia C, Ricciardiello O, Fedele L, Battista Candiani G. Increased frequency of complete hydatidiform mole in women with repeated abortion. Gynecol Oncol 1988;31(2):310-4.

13 Coulam C B. Epidemiology of recurrent spontaneous abortion. Am J Reprod Immunol 1991;26(1):23-7.

14 Parazzini F, La Vecchia C, Pampallona S, Franceschi S. Reproductive patterns and the risk of gestational trophoblastic disease. Am J Obstet Gynecol 1985;152(7 Pt 1):866-70.

15 Murdoch S, Djuric U, Mazhar B, Seoud M, Khan R, Kuick R, Bagga R, Kircheisen R, Ao A, Ratti B, Hanash S, Rouleau G A, Slim R. Mutations in NLRP7 cause recurrent hydatidiform moles and reproductive wastage in humans. Nat Genet 2006;38(3):300-2.

16 Deveault C, Qian J H, Chebaro W, Ao A, Gilbert L, Mehio A, Khan R, Tan S L, Wischmeijer A, Coullin P, Xie X, Slim R. NLRP7 mutations in women with diploid androgenetic and triploid moles: a proposed mechanism for mole formation. Hum Mol Genet 2009;18(5):888-97.

17 Hayward B E, De Vos M, Talati N, Abdollahi M R, Taylor G R, Meyer E, Williams D, Maher E R, Setna F, Nazir K, Hussaini S, Jafri H, Rashid Y, Sheridan E, Bonthron D T. Genetic and Epigenetic Analysis of Recurrent Hydatidiform Mole. Hum Mutat 2009.

18 Kou Y C, Shao L, Peng H H, Rosetta R, del Gaudio D, Wagner A F, Al-Hussaini T K, Van den Veyver I B. A recurrent intragenic genomic duplication, other novel mutations in NLRP7 and imprinting defects in recurrent biparental hydatidiform moles. Mol Hum Reprod 2008;14(1):33-40.

19 Puechberty J, Rittore C, Philibert L, Lefort G, Burlet G, Benos P, Reyftmann L, Sarda P, Touitou I. Homozygous NLRP7 mutations in a Moroccan woman with recurrent reproductive failure. Clin Genet 2009;75(3):298-300.

20 Qian J, Deveault C, Bagga R, Xie X, Slim R. Women heterozygous for NLRP7/NLRP7 mutations are at risk for reproductive wastage: report of two novel mutations. Hum Mutat 2007;28(7):741.

21 Slim R, Bagga R, Chebaro W, Srinivasan R, Agarwal N. A strong founder effect for two NLRP7 mutations in the Indian population: an intriguing observation. Clin Genet 2009;76(3):292-5.

22 Wang M, Dixon P, Decordova S, Hodges M, Sebire N, Ozalp S, Fallahian M, Sensi A, Ashrafi F, Repiska V, Zhao J D, Xiang Y, Savage P, Seckl M, Fisher R. Identification of 13 novel NLRP7 mutations in 20 families with recurrent hydatidiform mole; missense mutations cluster in the leucine rich region. J Med Genet 2009:jmg.2008.064196.

23 Tschopp J, Martinon F, Burns K. NALPs: a novel protein family involved in inflammation. Nat Rev Mol Cell Biol 2003;4(2):95-104.

24 Kinoshita T, Wang Y, Hasegawa M, Imamura R, Suda T. PYPAF3, a PYRIN-containing APAF-1-like Protein, Is a Feedback Regulator of Caspase-1-dependent Interleukin-1{beta} Secretion. J Biol Chem 2005;280(23):21720-5.

25 Gattorno M, Tassi S, Carta S, Delfino L, Ferlito F, Pelagatti M A, D'Osualdo A, Buoncompagni A, Alpigiani M G, Alessio M, Martini A, Rubartelli A. Pattern of interleukin-1beta secretion in response to lipopolysaccharide and ATP before and after interleukin-1 blockade in patients with CIAS1 mutations. Arthritis Rheum 2007;56(9):3138-48.

26 Milhavet F et al., Hum Mutat. 2008, 29(6):803-8;

27 Touitou I et al., 2004—Hum Mutat. 24(3):194-8;

28 Sarrauste de Menthière C, et al. 2003—Nucleic Acids Res. 31 (1):282-5.

29 Adzhubei I A et al. Nat Methods 7(4):248-249 (2010)

30 PCT application no. PCT/CA2006/001256 of SLIM, Rima, published as WO 2007/014463 on Feb. 8, 2007.

1. A method for diagnosing a predisposition for recurrent reproductive wastage in a female subject, the method comprising detecting one or more alterations in the sequence of a NLRP7 nucleic acid or encoded polypeptide in a sample from said subject, relative to the sequence of a wild-type NLRP7 nucleic acid or encoded polypeptide, wherein said one or more alterations are nonsynonymous mutations causing an amino acid changes at one or more positions corresponding to residues 250, 310, 311, 319, 340, 390, 413, 427, 430, 481, 487, 511, 659, 851, 872 and 931 in the NLRP7 polypeptide sequence of SEQ ID NO: 7, wherein the presence of said one or more alterations is indicative that the female subject has a predisposition for recurrent reproductive wastage.

2. The method of claim 1, wherein said nonsynonymous mutation causes: a Phe to Leu change at a position corresponding to residue 250 in the NLRP7 polypeptide sequence of SEQ ID NO: 7; a Gln to His change at a position corresponding to residue 310 in the NLRP7 polypeptide sequence of SEQ ID NO: 7; a Gln to Arg change at a position corresponding to residue 310 in the NLRP7 polypeptide sequence of SEQ ID NO: 7; a Leu to Ile change at a position corresponding to residue 311 in the NLRP7 polypeptide sequence of SEQ ID NO: 7; a Val to Ile change at a position corresponding to residue 319 in the NLRP7 polypeptide sequence of SEQ ID NO: 7; a Glu to Gln change at a position corresponding to residue 340 in the NLRP7 polypeptide sequence of SEQ ID NO: 7; a Glu to Lys change at a position corresponding to residue 340 in the NLRP7 polypeptide sequence of SEQ ID NO: 7; an Arg to His change at a position corresponding to residue 390 in the NLRP7 polypeptide sequence of SEQ ID NO: 7; an Arg to Trp change at a position corresponding to residue 413 in the NLRP7 polypeptide sequence of SEQ ID NO: 7; a Met to Thr change at a position corresponding to residue 427 in the NLRP7 polypeptide sequence of SEQ ID NO: 7; an Ala to Thr change at a position corresponding to residue 481 in the NLRP7 polypeptide sequence of SEQ ID NO: 7; a Gly to Glu change at a position corresponding to residue 487 in the NLRP7 polypeptide sequence of SEQ ID NO: 7; a Lys to Arg change at a position corresponding to residue 511 in the NLRP7 polypeptide sequence of SEQ ID NO: 7; an Arg to Leu change at a position corresponding to residue 659 in the NLRP7 polypeptide sequence of SEQ ID NO: 7; an Arg to His change at a position corresponding to residue 815 in the NLRP7 polypeptide sequence of SEQ ID NO: 7; a Tyr to Stop change at a position corresponding to residue 872 in the NLRP7 polypeptide sequence of SEQ ID NO: 7; and/or a premature termination of the NLRP7 polypeptide at a position corresponding to residue 931 in the NLRP7 polypeptide sequence of SEQ ID NO: 7.

3. The method of claim 2, wherein said nonsynonymous mutation is a nucleotide substitution.

4. The method of claim 3, wherein said nucleotide substitution is: a C to A substitution at a position corresponding to nucleotide 750 in the NLRP7 nucleotide sequence of SEQ ID NO:8; an A to G substitution at positions corresponding to nucleotide 929 in the NLRP7 nucleotide sequence of SEQ ID NO:8; a C to A substitution at a position corresponding to nucleotide 931 in the NLRP7 nucleotide sequence of SEQ ID NO:8; a G to A substitution at a position corresponding to nucleotide 955 in the NLRP7 nucleotide sequence of SEQ ID NO:8; a GAG to CAAAA substitution at positions corresponding to nucleotides 1018 to 1020 in the NLRP7 nucleotide sequence of SEQ ID NO:8; a G to A substitution at a position corresponding to nucleotide 1018 in the NLRP7 nucleotide sequence of SEQ ID NO:8; a G to A substitution at positions corresponding to nucleotide 1169 in the NLRP7 nucleotide sequence of SEQ ID NO:8; a C to T substitution at a position corresponding to nucleotide 1237 in the NLRP7 nucleotide sequence of SEQ ID NO:8; a T to C substitution at a position corresponding to nucleotide 1280 in the NLRP7 nucleotide sequence of SEQ ID NO:8; a G to A substitution at a position corresponding to nucleotide 1441 in the NLRP7 nucleotide sequence of SEQ ID NO:8; a T to C substitution at a position corresponding to nucleotide 1460 in the NLRP7 nucleotide sequence of SEQ ID NO:8; an A to G substitution at a position corresponding to nucleotide 1532 in the NLRP7 nucleotide sequence of SEQ ID NO:8; a G to T substitution at a position corresponding to nucleotide 1976 in the NLRP7 nucleotide sequence of SEQ ID NO:8; a G to A substitution at positions corresponding to nucleotide 2444 in the NLRP7 nucleotide sequence of SEQ ID NO:8; and/or C to A substitution at a position corresponding to nucleotide 2616 in the NLRP7 nucleotide sequence of SEQ ID NO:8.

5. (canceled)

6. The method of claim 5, wherein said nonsynonymous mutation is a nucleotide deletion.

7. The method of claim 6, wherein said nucleotide deletion is: a deletion of a GC dinucleotide at positions corresponding to nucleotides 930 and 931 in the NLRP7 nucleotide sequence of SEQ ID NO:8 and/or a TG deletion at positions corresponding to nucleotides 2791 and 2792 in the NLRP7 nucleotide sequence of SEQ ID NO:8.

8-52. (canceled)

53. The method of claim 1, wherein said recurrent reproductive wastage is hydatidiform mole, spontaneous abortion, blighted ovum, elective termination, choriocarcinoma or gestational trophoblastic neoplasia.

54. The method of claim 1, wherein said female subject is undergoing, or is a candidate for, assisted reproductive technologies (ART).

55. An oligonucleotide capable of specifically hybridizing, under stringent conditions, to the altered NLRP7 nucleic acid sequence defined in claim 1 and not to a corresponding wild-type NLRP7 nucleic acid sequence.

56-59. (canceled)

60. An antibody capable of specifically binding to the altered NLRP7 polypeptide defined in claim 1.

61. A kit for diagnosing a predisposition for recurrent reproductive wastage in a female subject, said kit comprising a reagent for detecting an alteration in the sequence of a NLRP7 nucleic acid or encoded polypeptide in a sample from said subject, relative to the sequence of a wild-type NLRP7 nucleic acid or encoded polypeptide, wherein said one or more alterations are nonsynonymous mutations causing an amino acid changes at one or more positions corresponding to residues 250, 310, 311, 319, 340, 390, 413, 427, 430, 481, 487, 511, 851 and 931 in the NLRP7 polypeptide sequence of SEQ ID NO: 7.

62. The kit of claim 61, wherein said reagent for detecting is an oligonucleotide capable of specifically hybridizing, under stringent conditions, to said altered NLRP7 nucleic acid sequence and not to a corresponding wild-type NLRP7 nucleic acid sequence.

63. The kit of claim 61, wherein said recurrent reproductive wastage is hydatidiform mole, spontaneous abortion, blighted ovum, elective termination, choriocarcinoma or gestational trophoblastic neoplasia.

64-67. (canceled)

68. The oligonucleotide of claim 55, which is capable of specifically hybridizing, under stringent conditions, to an altered NLRP7 nucleic acid sequence comprising: a C to A substitution at a position corresponding to nucleotide 750 in the NLRP7 nucleotide sequence of SEQ ID NO:8; an A to G substitution at positions corresponding to nucleotide 929 in the NLRP7 nucleotide sequence of SEQ ID NO:8; a C to A substitution at a position corresponding to nucleotide 931 in the NLRP7 nucleotide sequence of SEQ ID NO:8; a G to A substitution at a position corresponding to nucleotide 955 in the NLRP7 nucleotide sequence of SEQ ID NO:8; a GAG to CAAAA substitution at positions corresponding to nucleotides 1018 to 1020 in the NLRP7 nucleotide sequence of SEQ ID NO:8; a G to A substitution at a position corresponding to nucleotide 1018 in the NLRP7 nucleotide sequence of SEQ ID NO:8; a G to A substitution at positions corresponding to nucleotide 1169 in the NLRP7 nucleotide sequence of SEQ ID NO:8; a C to T substitution at a position corresponding to nucleotide 1237 in the NLRP7 nucleotide sequence of SEQ ID NO:8; a T to C substitution at a position corresponding to nucleotide 1280 in the NLRP7 nucleotide sequence of SEQ ID NO:8; a G to A substitution at a position corresponding to nucleotide 1441 in the NLRP7 nucleotide sequence of SEQ ID NO:8; a T to C substitution at a position corresponding to nucleotide 1460 in the NLRP7 nucleotide sequence of SEQ ID NO:8; an A to G substitution at a position corresponding to nucleotide 1532 in the NLRP7 nucleotide sequence of SEQ ID NO:8; a G to T substitution at a position corresponding to nucleotide 1976 in the NLRP7 nucleotide sequence of SEQ ID NO:8; a G to A substitution at positions corresponding to nucleotide 2444 in the NLRP7 nucleotide sequence of SEQ ID NO:8; and/or a C to A substitution at a position corresponding to nucleotide 2616 in the NLRP7 nucleotide sequence of SEQ ID NO:8.

69. The oligonucleotide of claim 55, which is capable of specifically hybridizing, under stringent conditions, to an altered NLRP7 nucleic acid sequence comprising: a deletion of a GC dinucleotide at positions corresponding to nucleotides 930 and 931 in the NLRP7 nucleotide sequence of SEQ ID NO:8 and/or a TG deletion at positions corresponding to nucleotides 2791 and 2792 in the NLRP7 nucleotide sequence of SEQ ID NO:8.

70. An antibody capable of specifically binding to an altered NLRP7 polypeptide comprising: a Phe to Leu change at a position corresponding to residue 250 in the NLRP7 polypeptide sequence of SEQ ID NO: 7; a Gln to His change at a position corresponding to residue 310 in the NLRP7 polypeptide sequence of SEQ ID NO: 7; a Gln to Arg change at a position corresponding to residue 310 in the NLRP7 polypeptide sequence of SEQ ID NO: 7; a Leu to Ile change at a position corresponding to residue 311 in the NLRP7 polypeptide sequence of SEQ ID NO: 7; a Val to Ile change at a position corresponding to residue 319 in the NLRP7 polypeptide sequence of SEQ ID NO: 7; a Glu to Gln change at a position corresponding to residue 340 in the NLRP7 polypeptide sequence of SEQ ID NO: 7; a Glu to Lys change at a position corresponding to residue 340 in the NLRP7 polypeptide sequence of SEQ ID NO: 7; an Arg to His change at a position corresponding to residue 390 in the NLRP7 polypeptide sequence of SEQ ID NO: 7; an Arg to Trp change at a position corresponding to residue 413 in the NLRP7 polypeptide sequence of SEQ ID NO: 7; a Met to Thr change at a position corresponding to residue 427 in the NLRP7 polypeptide sequence of SEQ ID NO: 7; an Ala to Thr change at a position corresponding to residue 481 in the NLRP7 polypeptide sequence of SEQ ID NO: 7; a Gly to Glu change at a position corresponding to residue 487 in the NLRP7 polypeptide sequence of SEQ ID NO: 7; a Lys to Arg change at a position corresponding to residue 511 in the NLRP7 polypeptide sequence of SEQ ID NO: 7; an Arg to Leu change at a position corresponding to residue 659 in the NLRP7 polypeptide sequence of SEQ ID NO: 7; an Arg to His change at a position corresponding to residue 815 in the NLRP7 polypeptide sequence of SEQ ID NO: 7; a Tyr to Stop change at a position corresponding to residue 872 in the NLRP7 polypeptide sequence of SEQ ID NO: 7; and/or a premature termination of the NLRP7 polypeptide at a position corresponding to residue 931 in the NLRP7 polypeptide sequence of SEQ ID NO: 7.

71. The kit of claim 61, wherein said reagent for detecting is an oligonucleotide capable of specifically hybridizing, under stringent conditions, to an altered NLRP7 nucleic acid sequence comprising: a C to A substitution at a position corresponding to nucleotide 750 in the NLRP7 nucleotide sequence of SEQ ID NO:8; an A to G substitution at positions corresponding to nucleotide 929 in the NLRP7 nucleotide sequence of SEQ ID NO:8; a C to A substitution at a position corresponding to nucleotide 931 in the NLRP7 nucleotide sequence of SEQ ID NO:8; a G to A substitution at a position corresponding to nucleotide 955 in the NLRP7 nucleotide sequence of SEQ ID NO:8; a GAG to CAAAA substitution at positions corresponding to nucleotides 1018 to 1020 in the NLRP7 nucleotide sequence of SEQ ID NO:8; a G to A substitution at a position corresponding to nucleotide 1018 in the NLRP7 nucleotide sequence of SEQ ID NO:8; a G to A substitution at positions corresponding to nucleotide 1169 in the NLRP7 nucleotide sequence of SEQ ID NO:8; a C to T substitution at a position corresponding to nucleotide 1237 in the NLRP7 nucleotide sequence of SEQ ID NO:8; a T to C substitution at a position corresponding to nucleotide 1280 in the NLRP7 nucleotide sequence of SEQ ID NO:8; a G to A substitution at a position corresponding to nucleotide 1441 in the NLRP7 nucleotide sequence of SEQ ID NO:8; a T to C substitution at a position corresponding to nucleotide 1460 in the NLRP7 nucleotide sequence of SEQ ID NO:8; an A to G substitution at a position corresponding to nucleotide 1532 in the NLRP7 nucleotide sequence of SEQ ID NO:8; a G to T substitution at a position corresponding to nucleotide 1976 in the NLRP7 nucleotide sequence of SEQ ID NO:8; a G to A substitution at positions corresponding to nucleotide 2444 in the NLRP7 nucleotide sequence of SEQ ID NO:8; and/or a C to A substitution at a position corresponding to nucleotide 2616 in the NLRP7 nucleotide sequence of SEQ ID NO:8.

72. The kit of claim 61, wherein said reagent for detecting is an oligonucleotide capable of specifically hybridizing, under stringent conditions, to an altered NLRP7 nucleic acid sequence comprising: a deletion of a GC dinucleotide at positions corresponding to nucleotides 930 and 931 in the NLRP7 nucleotide sequence of SEQ ID NO:8 and/or a TG deletion at positions corresponding to nucleotides 2791 and 2792 in the NLRP7 nucleotide sequence of SEQ ID NO:8.

73. The kit of claim 61, wherein said reagent for detecting is an antibody capable of specifically recognizing an altered NLRP7 polypeptide comprising an amino acid changes at one or more positions corresponding to residues 250, 310, 311, 319, 340, 390, 413, 427, 430, 481, 487, 511, 851 and 931 in the NLRP7 polypeptide sequence of SEQ ID NO: 7.


SEQUENCE LISTING (9 sequences)

SEQ ID NO: 1 - DNA, 23997 bp, Homo sapiens
gaaacacaggctggaagcaagacctgacctgagggaggtgagtgctggttcttgcatcgatttctttgtcttctcgtttaagggagaagaagctattggttgagtttccaccatagcccttcccaagccttaatggttggtgcgaggatgctggaaggatctttgatttttttttttttgagacggagtctccctctgtcgcccaggctggagtgcagtggcgtgatcttggttcgctgcaaactccgcctcctgggttcacccgccattctcctgcctcagcctcctgagtagctgggactacaggcacgtgccaccatgcccagctaatttttgtatttttagtagagacggggtctcaccatgttggccaggctggtcttgaactcctgactttaggtaatctgtctgcctcggcctcccaaagtgctgggattccaggtgtgagccaccacgcctggcctaatgtcttaaggacttctattcaaatatagtttagaggagttcaggagattgagactagcctgggcaacatggtaaaactctgtcattacaaaaaaatataaggccaggcacagtggttcatgcctgttatcccaacactttgggaggccgaggcgggtggatcacttgaggccagaagtttgagaccagcctgcccaaaatggtgaaaccctgtctctactaaaaacacaaaaattagccaggtgtggtggtgcatgcctgtaatcccagttacttggaaggttgaggcaggagaatagcttaaacctaggagggggaggttgcaatgagctgagatcgcgccactgcactccagcctgggagacagagtgcgactccgtctcaaaaaacaaagaaggccagaccttgtgctgtgtccaagctacttgtggggtgaggtgggaggatcacctgagccaggaggtgggggctgcaatgaggtgtgattgagccactgcactccagcctggatgagatgaagaccctgtttaaaaaaaaaaaaaagtaggctgggcgcggtggctcacgcctgtaatcccagcactttcagatcacctgaggccgggagtttgagaccagcctgaccaacatggagaaaccccatctctactaaaaatacaaaattagctgggcgtggtggcacatgcctgtaatcccagctactcgggaggctgaggcaggagaatcacttgaacccgggaggcggaggttgcggtgagctgagattgcgccactgcactccagcctgggcaaccagagtgaaatgctgcatcaaaaaaaaaaaaaaatgtaaatggccagatgcggtggctcacgcctgtgatcccagcactttgggaggccgaggcgggtggatcagctgaggtcaggagttcgaggccagcctggccaacatagagaaaccctgtctcggccgggcgcggtggctcacgcctgtaatcccagcactatgggaggccgaggcgggcggatcacgaggtcagaagatcgagaccatcctggctaacacggtgaaaccccatctctactaaaaatacaaaaaaaattagctgggcatagtggcgggcgcctgtagtcccagctacttgggaggctgaggcaggagaatggcgtgaacctgggaggcggagcttgcagtgagcccagatcgcgccactgcactccagcctgggtgacagagcaagactccatctcagaaaaaaacaagaaaccctgtctctactaaaaatacaaaaactagcccggcgtgatgcggtgcgcctgtaatcccagcttcttgagaggctgaggcactagaatcacttgaacctgggaggtggaggttgcagtgagtcgagattgtgccactgcactccagcctgggcaacagagggagactccatctcaaaaaaaagaaaaaaagaaaaaaatgtacttgggaaaaaaaatacttggccaggcctggtggctcatacctgtaatcccagcactttgggaggctgaggtgggcagatcacctgaggtcaggagttcaagaccagcctggccaacatggtgaaaccccgtttgtactaaaaatacaaaaaaaattaggtgtggtggggcatacctgtaatcccagctacttgggaggccgaggcaggagaatcgcttgaacccgggaagaggaggttgtggtaagcctcgcaccattgcactccagcctgggcgacagagcaagactttctgaaaaagaaaaaaaaaaccctgaatttttcttttcttttctttttttttttttttttgagagggagtctcactcgcccaggctggagtgcagtggcgcgatcttggctcactgcaagctccgcctcccaggttcaagccattctcctgcctcagcctcccaagtagctgggactacaggcgcccgccaccatgcccggctaattttttttttgtatttttagtagagacggggtttcaccgtgttagccaggatggtcttgagctcctcaccttgtgatctgcccgcctcggcctcccatagtgctgggattacaggcgtgagccaccgtgcctggccaaaaaaccctgaatttttctaagtacatcaagcgttgtcactgcaaaaacgaaaggcaactatatgaagcagtggatatgttaattagctggattgtggtaatcatttcactgtatatataacatcgctccatgctgtacactttgacaagtaaacgttgtatatattactttaaaaatacttaaaaaatagagacaaggtctccttgtgtcgcccaggctggtctggaactcctgggctctcatgctcttcctgctccatcctaaaataggatatatgtaattatacctcactgaaggggtggcctgcccctccacacctgtgggtgtttcttgtcaggtgggacgagagactgagaaaagaaagagacacagagacaaagtacacagaaagaaaagtgggctcaggagacccgcgccggccgggtctctgagttccttcagtatttattggtcattatctctaccatctcggagacggggatgtggcaggacaatagggtaacagtggggagagggtcagcaggaaaacatgtgagcaaatgtctgtgtcataaacaaggttaggaaatgtgctgtgccttgatgtgctcatacataaacatatctggtgcattaaagagcagtattgctgccagcatgtgtcacctccagccctaaggcggttttcccctatctcggtggatggaacataccatcgggttttacaccgagacattccattgcccagggacgagcaggagacagataccttcctcttaactgcaaaaaggccttcctcttatactaatcctcttcagcacagaccctttacgggtgtcgggctggggtacggtctggtctttcccttcccacgaggccttatctcaggctatcacatggggagaaactttggacaatacctggcttttctaggcagaggtccctgcagccttccgcagtgtattgtgtccctgggtgcttgagattagagagtggtgatgacttttaacaagcatgctgccttcaagcatctgtttaacaaagcacatcctgcatagccctaaacccacgtgtgacacagcacatgtttctgggagcacagggttggggctagggttacaggttaacagcatctcaaggcagaagaatttttcttagtacagaacaaaatggagtctcttatgtctatttctttctacatagacacagtaacagtctgatctgtcctccttttccccacacgtcacagctggggaaaaagatttgctgttccctccaagggtaaagctgtccacctctatcagcacccgggcttggcaagtcactttttctgttatttattttccaggctgcctcttccccccgcccccccaacccagacggagtctcgctctgtcgcccaggctggagtgcggtggcgcgatctccgctcactgcaagctccgcctcccgggttcccgccattctcctgcctcagcctcccgagtagctgggactacaggcgcccgccaccacgcccggctaattgtttgtatttttagtagagacggggtttcaccgtgttagccaggatggtctcgatctcctgacctcgtgatccgcctgcctcggcctcccaaagtgctgggattacaggcgtgagccaccgcacccggccattagttacttacttttgagacagggcctcactctgtcacccaggctggcgtgcagtggctggctcactgcaacctccaaatcgtaggctcaaacaatcctcctgtgtcagcctcccaagtatctgggactacgggtatgttccaccaggcctggctaagttttttttttttgagatagagtttcgctcttgttgcccaggctggagtacaatggcgctatctcagctcactgcaacctccgcctcctgggttcaagcgattctcctgcctcagcctcccacgtacctgggattacaggttcctaccactacacttggctagcttttgtatttttagtagagatggggtttcaccatgtgggccaggcgggtctcaaactcctgacatcaggcgatccacctgcctcagcctcccaaagtgctgggattccaggcctgagccaccatacccggccagtacagttatatttatatctgtcctcttgctatttgttttcaatgtgtcattcagtggtgggctgaaatgttaaacaagtggctctgagggttggtggcgaggaagtcttggtttgtagtgtttgctgatttgtttttttgtttgtttgagacagagtcttgttcttgttgccgaggctcgagtgcaatggcgtgatctcagatcatgcaacctccacctcccaggttcaagtgtgattctcctgtctcggcctcctgagtagctgggattacaggcacccgcctgtaatttctgtatttttagtagagatggggtttcgccgtgttggtcaggctggtcttgagctcccgacctcaggttatccacccgccttggcctcccaaagtgctgggattacaggcgtgagccaccgcgccctgccgtgtttgctgatttctgtggcataaacactccccttgtgattttgtactatcagtgtgaaatcacagcccatggacgttggtataggtacatataggaagcccccattaggcagcacgggctggccctagcataccactgacccttcattctttgtattcttttttttttttttttttttttttttgagacggagtctcgctctgtcgcccaggctggagtgcaatggtgagatctctgctcactgcaagctccacttcccgggttcacaccattctcctgcctcagcctcccgagtagctgggactacaggtgcccgccaccacgccctgctaattttttgtatttttttagtagaggcaggggtttcactgtgttagccaggatggtctcgatctcctgatatcgtgatccatccgcctcggcctcccaaagtgctgggattacaggcgtgagccaccgtgcccagccttttgttcgttctttttaccaagttagccaggctggtctcgaactcctggccgcaggcgtgagccaccgtgctgggccagattttcagtctcttaattcagtctttggaatattttaccactcactgtacagcaggaacagtcttgttcttggcacacaggaaactgtggtttcatttaatgatggtaactcgtgaactgtttttccttttttccccccagttcttcagccttaacctaaggtctcatactcggagcactatgacatcgccccagctagagtggactctgcagacccttctggagcagctgaacgaggatgaattaaagagtttcaaatcccttttatgggcttttcccctcgaagacgtgctacagaagaccccatggtctgaggtggaagaggctgatggcaagaaactggcagaaattctggtcaacacctcctcagaaaattggataaggaatgcgactgtgaacatcttggaagagatgaatctcacggaattgtgtaagatggcaaaggctgagatgatgggtaagtagaacctggggtgtcctggtcatttttttttttttttttttttttttgagatggagtctcgttctgtcgcccaggctggagtgtaaggctggagtgcagtggcgagatctgggctcactgcaacctccgcctctgggttcaagtgattctcctatctcagcctccggagtagctgggattacaggcgtgtttcaccacacctggctaatttttttttttttgtatttttagtagagatggggttttgccatgttggccaggctggtcttgatctcctgaccttgtgatccgcccacctcagccttccaaagtgctgtgattacaggcatgagccaccatgcctggctgacactttatgtacaataatgtctgatttacgaagtgtaaattactgtgtcaggcttacatctaagtattttacagaggacggacaggtgcaagaaatagataatcctgagctgggagatgcagaagaagactcggagttagcaaagccaggtgggtaaatacggtcctatggtcatgagtttggtgtttgagagcatgcaaggtgcatcacttcttcctggttttattcatttctggtagttttttttttttttgagacggaatcttgctctgtagcccaggctggagtgtagtggctccgtctctgctcattgcaacctctgcctcccgggttcaagcaattctctgcctcagcctcctgagtagccgggattacaggcggccgccactacccccagctaatgttttgtatttttagtagagatggggtttcactatcttggccaggctggtcttgaactcctgacctcaagtgatccacccaccttggcctcccaaagtgccgggattacaagcatgagacaccgtgcctggccctcatttctggtacttgacaaaataattcagaaaatcatcatcatcaacctcaactgtcctatgggctgtcactgcaggtgaaaaggaaggatggagaaattcaatggagaaacagtctttggtctggaagaacaccttttggcaaggagacattgacaatttccatgacgacgtcactctgagaaaccaacggttcattccattcttgaatcccagaacacccaggaagctaacaccttacacggtggtgctgcacggccccgcaggcgtggggaaaaccacgctggccaaaaagtgtatgctggactggacagactgcaacctcagcccgacgctcagatacgcgttctacctcagctgcaaggagctcagccgcatgggcccctgcagttttgcagagctgatctccaaagactggcctgaattgcaggatgacattccaagcatcctagcccaagcacagagaatcctgttcgtggtcgatggccttgatgagctgaaagtcccacctggggcgctgatccaggacatctgcggggactgggagaagaagaagccggtgcccgtcctcctggggagtttgctgaagaggaagatgttacccagggcagccttgctggtcaccacgcggcccagggcactgagggacctccagctcctggcgcagcagccgatctacgtaagggtggagggcttcctggaggaggacaggagggcctatttcctgagacactttggagacgaggaccaagccatgcgtgcctttgagctaatgaggagcaacgcggccctgttccagctgggctcggcccccgcggtgtgctggattgtgtgcacgactctgaagctgcagatggagaagggggaggacccggtccccacctgcctcacccgcacggggctgttcctgcgtttcctctgcagccggttcccgcagggcgcacagctgcggggcgcgctgcggacgctgagcctcctggccgcgcagggcctgtgggcgcagatgtccgtgttccaccgagaggacctggaaaggctcggggtgcaggagtccgacctccgtctgttcctggacggagacatcctccgccaggacagagtctccaaaggctgctactccttcatccacctcagcttccagcagtttctcactgccctgttctacgccctggagaaggaggagggggaggacagggacggccacgcctgggacatcggggacgtacagaagctgctttccggagaagaaagactcaagaaccccgacctgattcaagtaggacacttcttattcggcctcgctaacgagaagagagccaaggagttggaggccacttttggctgccggatgtcaccggacatcaaacaggaattgctgcaatgcaaagcacatcttcatgcaaataagcccttatccgtgaccgacctgaaggaggtcttgggctgcctgtatgagtctcaggaggaggagctggcgaaggtggtggtggccccgttcaaggaaatttctattcacctgacaaatacttctgaagtgatgcattgttccttcagcctgaagcattgtcaagacttgcagaaactctcactgcaggtagcaaagggggtgttcctggagaattacatggattttgaactggacattgaatttgaaaggtaagaactgttttcccatcccacgctccactaggaagaggccagcgtctcctttgccctgtcgcttactgtcagaatttccctctggctggacttctttccagcttcatgttcaacgtggagacacgacttggcaattaggaattggggctttttatttttgagacggagtctcgctctgtcccccaggctggagtgcagtggcgcgatcttggctcactgcaacctccgcctcccgggttcaagtgattctcctgcctcagcctcccgagtagctgggactatgggcgtgcaccaccttgcccggttaattattttatttttttgtagagatgggggtctcagtttctagcccaagttggtcttaaactcctgggctcaagtgatcttcccactttggcctagcaaagtgttgggattacaggcatgagccacctcactcagccttatctattattttattttttttgtaaaacttaagatctatactggtagcaaagcatgtgatgcaatattgtttactatagacactgttttaggttggtgcaaaagtaattgtggtttttgccattgaaatgtggtttgcagatgcccatctcaccatgcaggtactagtcctaagagatgaacgtgtgttctcctgcaggtgcacttacctaaccattccgaactgggctcggcaggatcttcgctctcttcgcctctggacagatttctgctctctcttcagctcaaacagcaacctcaagtttctggaagtgaaacaaagcttcctgagtgactcttctgtgcggattctttgtgaccacgtaacccgtagcacctgtcatctgcagaaagtggagtaagtagaagctcatcttgcaaggaagaccctgaacgatgactaagcttcttgtacttttgttttttaaatttggaaatgtgctgtttcatctccatgtatttggggattttccagctgtctttttttttttttttttttttggtgagacggagatttactcttgttgcccaggctggagtgcaatggcgcgatctcagctcactgcatcctccacctcccaggttcaagcaattctcctgcctcagcctcccgagtagctgggattacaggcatgtgccaccttgcccggctaattttgtacttttagcacagacaggttttcaccgtgttgcccaggctgatctcgagctcctgacctcaggtgatttgcctgcctcggccttccaaagtgctgggattataggcatgagccgctgcacctggccccttttttattttttattttttctgagacagagtttcactctgtcacctaggcgctggagtgcaatgacttaatcttgtgtttttagtagaggtggaattttctccatcttggccaggcttgtctcgaactcctgacctaaggtgatgcgcctgcctcggtcttcgaaagtgctgggattacaggcatgagccaccatgcctggccccagctatcttttttttggtttgttttgttaccaaaacaaaccaaaaagtaggtacaagtacaggttagttacacaggtaaccgtgtgtcataggagtttgttgtacagattattttgtcacccaagtattaagcctagtaccccttagttgtttttcctgatcctctgcttcttgacttttttttttttttttgagacagtctcgctatgttccccaggctggagtgcagtgcagcaatctcggctcactgcaagccctgcctcccgggttcatgccattctcctgcctcagcctcccgagtagctgggactacaggcgcccgccaccacgcccggctagttttttgtaattttagtaaagacggggtttcaccgtgttagccaggatggtcttgatctcctgacctcgtgatccacccgcctcggcctcggcctcccaaagtgctgggattacaggcgtgagccaccacacccggcgaatttttttttcttttgagatggagtcttgctctgttgcccaggctggagtgcagtggtgcggtctcggctcactgcaacctctgcctcctggattcaagtgattctcctacctcagcctcccgaatacctgggactacaagcatgcccctccatgtgcagctaatttttgtatttttagtagagacggggcttccccatgttggccaggctggtctcgaactcctgacctcaggcgatctgcctgcctcggccccagctaatttattttttgtagagatggagtttcaccatgttgcccaggttggtctcagactcctgacctcaggttatcctcctgcctcagcctcccaaagtgctggggttacagacacgagccactgcacccggccaagaacttctaataatttctaaatgtgaaacagctttttgtttatacatgcctccacacaatgtgagtattaatcactccaagtggaatctcttctgcttttccctaggattaaaaacgtcacccctgacaccgcgtaccgggacttctgtcttgctttcattgggaagaagaccctcacgcacctgaccctggcagggcacatcgagtgggaacgcacgatgatgctgatgctgtgtgacctgctcagaaatcataaatgcaacctgcagtacctgaggtgggtctcacggtcacggctctccccagcacctggagtccactgcaccgtgttgctgggggatctaggaaaaagggtaaccactccagatgccgtcccagacagggaatgtattcctcaaacaggcctgtgtgggggagtcggcctctcctctttcccccaccagcttgtcttctgtgttgcataaccagctatccatgcaaagaaacaccccgaattctgtgctgggttccagctttagggacatgctattcctgactgcaccttgcctaattgttgggattgagagcagtggcccccagccttttctgcaccgcgggccggttttgcacaagacagttttttccacagacgggtttgggggtagttttgggatgaaactgttcgatctcagatcaggcacaggagctaatcgttggtgcctgatcctatggagtgcatgatcctcgcactttgggagcctgaggagaatggatcatcaatctcagatcatcaggagttaggtattcataaggagcatgcaaccttctctgcactcaatgagaatcttttttttttttttttttctttgagacagttttattcttgtcacccaggctggagcgcagtggcgcgatctcgttcactgcaacctccgcctcctgggttcaagcagttctgcctcagcttcccgagtagctggggttacaggcgtgcaccaccacgcctggcaaatgtttgtatttttaatagagacagggtttcaccatgttggccaggctggtctcgaactcctgacctcaagtgatccgcctgtctcggcctcccaaagtgctaggattacaggcatgaaccactgcgcctggccaggataaaatttttattttgagtattaagcatcaatttgccccttctagtcccagctacagtggatgctgaggtgggaggatcatttgagcccaggagacaggttgtggtgacctgtgatcatgccactgcactccagcctgggcaacagagcgagatcctgtctcaaaaaaaaaatttttttttcccccctgcaaaatcatccacacaggccgttttggtgaaacattgcacagaattgtattacaatctcttggagaagtggctggatgttaccctaatggccatggggatacttgaagaagcagaggcaacattagatctctccagtaattcaggccagggttggaggcatgagtagaatgagataaaccaaagacataatgtcttgggaagtgaagcagaagaagctgatctgggccaggcgcggtggctcacacctgtaatcccagtacttcggtaggccaaggtgggtggatcacctgaggtcaggagttcaagaccagtgtggccaacatggtgaaatcccgtctctactaaaaatacaaaaattggcgaatgcctgtaatcccagctacttcggaggctgaggcaggagaatagcttgaacccgggaggcggaggctgcagtgaggtgagatcacgcctttgcattccagactgggcaacagagtgaaactctgtctcaaaaaaaaaaagctgatagggtatactctgtcctcccagaagaatgacttttcccactcttttcacaggttgggaggtcactgtgccaccccggagcagtgggctgaattcttctatgtcctcaaagccaaccagtccctgaagcacctgcgtctctcagccaatgtgctcctggatgagggtgccatgttgctgtacaagaccatgacacgcccaaaacacttcctgcagatgttgtcgtaagtctcctcttcccatgggcagctctggtttagttctggggctatagaagagaaagggtaacacctgacttactgcgccacccacgtggcgcctcttgctgaaataaacacctgcttcaggcccggcacggtggctcctgcctgtaatctcagcagagaggtgggcggatcatctgagttcaggagttcgagaccaacctggccaacatggtgaaaccctgtttctattaaaaataccaaaaacaggccgggtgcggtggctcatgcctgtaatcccagcacgttgggaggccaaggcggggagatcacgaggtcaagagatcgagaccatcctggctaacatggtgaaaccccgtctctactaaaaaatacaaaaaattatccaggtgtggtgggcgcctgtagtcccagctactcaggaggctgagtcagcagaatggtgtaaacctgggaggcggcgattggcagtgaaccgagatcgcgccactgcactccagcctgggcgacagagcgagactccgtctcaaaaacaacacctgtgtcctgtgatggctccaggtggaccgctgcatcttggccttctcgccttcctgctcttttgtggccatgatgactcccacaggacagagggcaggggatgaacaggaagggctgaagctgagtaccctagcatgtggacatcactgagcaggttggagttgtggaaatgttctcatccttctaccatttgtttcatatttttgcaggttggaaaactgtcgtcttacagaagccagttgcaaggaccttgctgctgtcttggttgtcagcaagaagctgacacacctgtgcttggccaagaaccccattggggatacaggggtgaagtttctgtgtgagggcttgagttaccctgattgtaaactgcagaccttggtgtaagtccctgctgggtgtgtgtgtgtgtgcacatgaattcaagcaggagagacatgaaagtacttgttaattcatttcaaatgtaacttttaaaaacctggtaagaattaaagaacaggcagaggccaggcgtggtggctcatgcctgtaatcccagcactttgggaggccgaggcgggtggatcatgaggtcaggagatggagaccatcctggttaacatggtgaaaccctgtctgtactaaaaataccaaaaattagccaggtgtggtggcggatgcctgtagtcccagctacttgggaggatgagacaggagaatggcgtgaacctggaaggcggaggttgcagtgagccgagatcgcaccactgcactccagcctgggcgacagaacaagactccttctcaaaaaaacaaagaaacaaaaaaaaccaggcagatacaggtagaaacatgttaatatttgcatgtcagcagagcctcttcctgctatgaaggaagatttgagatgagtagttggttctcggatctgatgctttgtgtgtgttctttcaaattcctatgacatagtactgcctgctattggaggtagattgagttatgtggtagggccagtggcacctttttttaaacttttatttccataggttattggggaacaggtggtgaatggtgggcagatcacctaaggttcgagaccagcctggccaacatggtgaaaacccatcgctactaaaaaatacaaaaattaaccaggcttggtggtgcgtgcctatagtaccagctactcagaaggctgaggtaggagaatcgcttgaatctgggaggcagaggctgcagtgagctgagatggcgccactgcactccagcccgggcgacagagtgagactccgtctcaagaaaaaaacaaaaaaaaactcaacaaaaatccttatttgtaaaagacataggtggcaggttggaattgacccacgaactatagttggctgaatcttgttatatggaaagaagcccagcgtgagctacctgttcacattaaaattatggttagaaaaatattcaagagattgcatagggttgaagacctgttcctgttcagaaattctagctagtggtcatttctgagattcatttttttttttttggatgaagtctcactctgtcgcccagactggaatgcagtggtgtaatcttggctgactgcaacttctgcctcccaggttcaagcgattctcctgcctcagcctcccaagtagctgggattacaggtgccctccaccatgcctggctaatttttgcacttttagtggagatgaggtttcaccatgttggccaggctggtcttgaactcctggccttaagtgatctgcctgcctcggcctcccaaagtgctggcgttccaggcatgagccactgtgcctggcttagaataactattgttaaacaaacagtcacctacctgatcgttatacgaagtgtacctgcaccaaaacatcacactatacccctatatatgtagaatgtgtcagttaaagacaaaacttaaacatgaaataaaatgacagggaaagtgaaatttccataatctaaccacgcagaaaataagtgacccagggctcagatcctgtcctgggtcggtctgaacccagagcctaagctgttgtcccaggcagagctggaaatggatggaatcagaaggccatttggatgttttttttttttttttaacagtctctctctgtcaccaggctggagtgcagtggtgcgatcttggctcactgcaacctccgcttcctgggttcaagtaattctcctacctcagcctcctgagtagctaggattacaggcatgggccgccacacctggctaatttttttttttttttgagatggagtttcgctcttgcccaggctggagtgcaatggtgcaatctctgctcaccacaacctccgtctccccagttcaagagattctcctgcctcagcctcctgagtagctgggattacaggcatgtgccaccacacctggctaattttgtatttttagtagagacgggtttctccatattgcttaggctggtcttgaactcccgacctcaggtgatctgtctgcctcagcctcccaaagtgctgagattacaggtgtgagccatcgtgcccagctaatttttgtatttagtaaagatggggtttcaccactttggccaggctggtcttgaactcctgatcttgtgattcacccaccttggtctcccaaagtgctgagattacaggtttgagccaccgcgcccggcccgatttttgtattttttagtagagatggggtttcaccatgttggccaggctggtcttgaactcctgacctcaaatgatctgcccgtcttggcctcccactgctgtgattataggcgtgagccactgtgcccggcccatttgcatgcttttatgtgcaagcccacctggaagtatatagctccagttcatgggtcaattcctacctgccacctatgttttatataaatactttttgttgttgttgttgttttcttgagacggagtctcgctctgtcgcccgggctggagtgcagtggcgcgatctcagctcactgcagcctctgcctcccggattcaagcgattctcctgcctcagtcttctgagtagctggcactacaggcgtgcaccaccaagtctggttatataggtggcgggcacctataatcccagctacttgggaggctgaggcagaagaatcgcttgaacctgggaggcagaggttgcagtgagccaagagtgcagcactgcattccagtatataagtggaaggtatatagtgttggaaataactgcttcacagggcgttagccagagggataacaggcttctcttcctttgattatcctgtaggttacagcaatgcagcataaccaagcttggctgtagatatctctcagaggcgctccaagaagcctgcagcctcacaaacctggacttgagtatcaaccagatagctcgtggattgtggattctctgtcaggcattagagaatccaaactgtaacctaaaacacctacggtaggcgattttctttttcttctttctttctttttttgagacagggtcttgctctgtcccccagcctggagtgcagtggggtgattacggctcactgcggcttcggtcttccaggcttgatcggttctcccacctcagcctcctgagtagctggctctacaggcatgtattaccatggccaggtaactgttttctgtagagatgaggtcttgtcatctttcccgggctggttttgaattctggtgctcaaggaatcctcccacctcggcctcccaatgtgctaggattacaggcatgagccatcatgcctggcctcatttttaaagtgtttggaaatctggaaatccttaatttctatgttttctttttttttttttttttttgagacggagcctcgttctagttgcccaggctggagtgcagtggcgcgatctcggcttactgcaacctcttcctcccgggttctcgctattctcctgcctcagcctcctgagtagctgggactacagatgcccgccaccgtgcctggctaattttttttgtatttttagtagagatgggtttcacagtgttagccaggatggtctcgatctcctgacctcatgatctgcccgccttggccttccaaagtgctgggattacaggcgtgagccaccacgcccggccaatttctatgttttcaatatctcagactgtatcacttcggatccagttttaagatcaaacccctccagaaactgaatatatgtgggtgggcacttctaaagtcaggtagagggcctggagaagtgaaatatatataacaatggcccccagtgacctggacttcagcagcatgctgcttctgctgggatccagtaatcaggaagcagtgagcctgccccacctcataaacccagggaaccataggtgggataccacccccagaaaatgcaaagtctccacaaatggaatggcgagctcttcatcacttctctccccaaagtttgtcagttgcatctcttggatgcaacctattttccaactagaatctgcaatcctaatgcaaagagaatctgcacgtcattactacttagctttgctgtagagtaaagaaaaaaaacactagaacacagggtactttttttcttttttcagacagagtctcgctttgtcacccaggctggagtgcagtggtgcgatcttggctcactgcaacctcagcctccaaggttcaagcgattctcctgattgagctgagtagttgggattacaggcgtgcaccaccatacccagctaatttttgtatttttagtagagaccaggtttcaccatgttagccagactggtctcaaactcctgacctcaagtgatccacctgcctcaacctcccaaagtgctgggattacaggcatgagccaccattcctggcctcctgaagtttcttaacccatccccctgaggaatatttcaagcctcaagccagaccgtgatacctttatttccaaagactcaaaagctcaatgcaaacgggtggattacctggtgtcttgttcctgtaatctcagctatgactgtaatcctagattctcgggaggctggggcaggagaatcgcttgaacccaggaggcggaggttgcagtgagccgagatcacgccattgcactccagccttggcaacaagagtgaaactctgccttaaaaaaaacaaaaccaaaggcttctacagtggcctacagggccttatgggggatcctcgtgtaagttatgagccataaatcattctactttctcactagctcagtattttatttacaagattccctcccccagttagcatgctggttcatgatctaccatccttcagtttctttcctcatatcactttccaaaagaggacttaaatgaccagcataagtctagccaatcaatgcctctctgtttgacttacctctaccctgtttattttaataccatcatccattgtcttcaatagaacatatcgagatgtctgctgtcactaaaaactctgaggacaaggatttcttctgctcactcccctctgcctttcctcactactggagccccagcaaatatgctgcttgtttttttgttttgttttgtttgagaccaagtctcactctttcacccaagctggaatgcagtggtgatatgttggctaactacaacctctgcctcctggttcaggcgattctcctgcctctcgagtagctggaattataggtggttccaccatacctggctaatttttgtattttcattttatgttatatatttgtgagatggagtctcattctattgcccaggctggagtgcagtggcgcaatctgggctcactgtaacctccgcctcccaggctgaagcgattcttgtgcctcagcctcccaagtagctagcattaaaggcacacaccaccatgcatggctaattttttgtagagatggggttttgccatgttggcctggctggtctcgaactcctgacctcaggtgatctaccctcctcggcctcccaaggtgctggggctacaggtgtctgtccccacgccctgcctaatctttgtatttttagtagagatggggtttgaccgtgttggcaaggctggtctcgaacacctggcctcaagtgatccacccgccttggcctcccgaagtgttgggattacacgcttgagccactacctgctcagtgaatgcgtggatttccatgttcttcctcaacagcctctggagctgctccctcatgcctttctattgtcagcatcttggatctgctctcctcagcaatcagaagcttgaaactctggacctgggccagaatcatttgtggaagagtggcataattaagctctttggggttctaagacaaagaactggatccttgaagatactcaggtatgggttttttgttttgttttgttttgttttttgtttttgtttttttgagatggagtcgtgctctgtcattcaggctggagtgcagtggcgcaatcttggctcaccgcaacctctgcctctcaggttcaagcaattctcctgcctcagcctcatgagtagctgggcctagaggcatgccaacatgtccagctaatttttttctttttctttttttttttttgagacggagttttgttcttgtagcccaggctggagtgcagtggtgcgatcttggctcactgcaacccccacctcctgggttcaagcgattctcccaccttggcctcccaagtagctggaattacagatgcctgccaccatgcctggctaattttttagtagagaggggtttcaccatgttggccaggctagtcttgaactcctgacctcaggtgagccacctgcctcggcctcccaaagtggtgggattacagaggtgagccattgcacccggcctttttggtttttgctttttgggatggagtctcactgttgcccaggctggagtgcagtggcgcgatcttgactcactgcagcctccttctcacaggttgaagcgattttcctgcctcaacctcctgagtagctgggattacaggtacacaccaccacagctggctaattttttttttttttttttttttttaaagacagagtctctctctgtcccccaggctggagtgcagtggcgctatctcggctcagtgcaacctctgcctcctgggttcaagtgattctcctgcctcagcctcctgagtagctaggattacagtcgctcgccaccacacccagctaatttttgtatttttagtagagatggggttttgccatgttggccaggctggtctcgagctcctgacctcaggtgatcttctcgccttggcctcccaaagtgctgggattacaggcatgagccactgcacctggccaatttttgtagtttttagtagagatggggtttcaccatgttggtcaggttggtctcaaactcccaacctcaggtgatccacctgcctcagcctctcaaagtgccgggattacaggcgtgagccactgtgctcggccctgggatggctgtttcacatggtgaatttcccatgcagagaagagtttttttgggagtgtgtgtactctttgtagggatcaacttaaggcatctttctatagcacactcctagcttaggagataatttaaaaattagatacttttctaaaatgctctgtgaattgaatattgtccaactttcccccaaaacacttagtcctaggcatactgagagtttaaatcatcctggagtacagactggaagcttgtgtgtatgtgtgtgcatgagcacacacacacacacacacacacccctaatcattatatccaaaaataggtagttcccagagctgtcctgggtcttagcttttcagaagatcgtcctacagatgctcccttagttgtgacccgtgtatatcttttcaatgacttatttgtattttttatttttttttgagacggagtcttttttttgagacggagtctgtctttttttttgaatctgtcttttttttgagacagagactccagtctctgtcgcccaggctggagtgaagcggtgcgatctcggctcactgcaagctccacctcccgggttcacgccattctcctgcctcagcctcccgagcagctgggactacaggcgcccgccaccacgcccggctaattttttgtatttttagtagagatggggtttcactatgttggccaggctggtctcgaattcctgacctcaggtgatctgcccacctcggcctcccaaagtgctgggattacaggcgtgagccaccgcgcccggcctcagtgacttattttaacgtaatctacctttagtttcttcttgcctttgtcttttcttttctgagacaacgttttgctctgctgcactgtgtggccgtgttgccgaggttctcaaactcctggcttcaaacgatcctcctgtcttggcctcacaaagtacccggattgcaggcgtgagccactgtgcacagcccacttgtcttattcaagagttattttagttgtagagatgatacgcatgtaaactgcttcatgatgcccagtgttgcattattggaacgctaagcatgtgggagttatttatatcctgctcaaggtacgatttttcacacgtctgcagttcaaataattgtaacctctggcataaatgggttaaggttttaggggtatatcatgaaacttgagctaaatagtgtcatgcttctcttgttggtgggaccgaggtctgtaatgccaccaaggactattggtgacaaatctctagccccctgtggtctcttatgtcatatgtttggggcgtatttcttttctcattcctcagttcctcctttgggaggccaaggtgggaggattgtttgaggccaggagtttgagaccagcctgggcaacatagcaagccagtgtctccacaatcaccacccctcatgttcacatacacaggcttgcatgctgcagccacgttagagccaagtttgctatcattaaccctggggttcactctggcattctcttagttctactgaaggtttgatttgccactattttttatttatttatttggaggcagagtctcgctctgtcacccgggctgcagtacagtggtgcggtattggctcactgcaacatctgcctcccaggttcaaagcgattctcctgtctcagcctcctgagtagctggtattacagttgtctgccaccatgcccagctaatttttgtatttttagtagagacggggtttcactatgttggccaggctggtctcgaattcctgacctcaggtgatctgcccgcctcggcctcccaaagtgctggaattataggcgtgagtcaccgtgcaccagcctgattatctattttttaaatttattttttaaaggcatgttttactctgttaccaggctggagtgcagtagggcaatctctagctcgttgcaacctccgcctcctgggctcaagtgatcctcttgcctccgcctcccgagtagctgggactataggcgtgcaccaccattcctggctaactttttctatttttggtagagacagggtttcaccgtgttgcccaggctggccttgaactgcggagctcaagcaatctgcctgccttggcctcccaaagtgctgggactacaggtgcgagacaccgtgcctggccataatcttttttttcttagacttataaggatccccattgtgtgggtctaaatttctttttagaaaacttttctgactgggtgctgtggctcacatctgtaatcccatggctttgggaggccgaggtggatggatcacttgaggccagaagttcgagaccagcctggctaacatgtcgaaaccccatctctactgtaaatacaaaacttagccaagcgtggtggtgcacacctgtaatcacagttactcaggagcctgaggcatgagaattgcttgaacttgggagctggaggttgcagagagccaagatggcaccactgtaccccagcctgggcaacagagcaagaccctgtcccccagaaaatcccaaaaacgtttcctgctttgagtgtttgaaaacagatattcaggcatcctgggtagttgagaatgaatttctgggaacatttgtgttctctgatccctccaggttgaagacctatgaaactaatttggaaatcaagaagctgttggaggaagtgaaagaaaagaatcccaagctgactattgattgcaatgcttccggggcaacggcacctccgtgctgtgactttttttgctgagcagcctgggatcgctctacgaattacacaggaagcgggattcgggtctctaagatgtcttatgaatgcaggtcagagggtcacatgttaacactagagtctgtcgagaggtaggatttgacactggttttctcactatttttgggagattctgcacgagtcacgcacccccttcacatgacgctatgtactttctcacagggataataaagttagagcactctcgttgca

SEQ ID NO: 2 - DNA, 3250 bp, Homo sapiens
  CDS (77)..(3019)
gaaacacaggctggaagcaagacctgacctgagggagttcttcagccttaacctaaggtctcatactcggagcactatgacatcgccccagctagagtggactctgcagacccttctggagcagctgaacgaggatgaattaaagagtttcaaatcccttttatgggcttttcccctcgaagacgtgctacagaagaccccatggtctgaggtggaagaggctgatggcaagaaactggcagaaattctggtcaacacctcctcagaaaattggataaggaatgcgactgtgaacatcttggaagagatgaatctcacggaattgtgtaagatggcaaaggctgagatgatggaggacggacaggtgcaagaaatagataatcctgagctgggagatgcagaagaagactcggagttagcaaagccaggtgaaaaggaaggatggagaaattcaatggagaaacagtctttggtctggaagaacaccttttggcaaggagacattgacaatttccatgacgacgtcactctgagaaaccaacggttcattccattcttgaatcccagaacacccaggaagctaacaccttacacggtggtgctgcacggccccgcaggcgtggggaaaaccacgctggccaaaaagtgtatgctggactggacagactgcaacctcagcccgacgctcagatacgcgttctacctcagctgcaaggagctcagccgcatgggcccctgcagttttgcagagctgatctccaaagactggcctgaattgcaggatgacattccaagcatcctagcccaagcacagagaatcctgttcgtggtcgatggccttgatgagctgaaagtcccacctggggcgctgatccaggacatctgcggggactgggagaagaagaagccggtgcccgtcctcctggggagtttgctgaagaggaagatgttacccagggcagccttgctggtcaccacgcggcccagggcactgagggacctccagctcctggcgcagcagccgatctacgtaagggtggagggcttcctggaggaggacaggagggcctatttcctgagacactttggagacgaggaccaagccatgcgtgcctttgagctaatgaggagcaacgcggccctgttccagctgggctcggcccccgcggtgtgctggattgtgtgcacgactctgaagctgcagatggagaagggggaggacccggtccccacctgcctcacccgcacggggctgttcctgcgtttcctctgcagccggttcccgcagggcgcacagctgcggggcgcgctgcggacgctgagcctcctggccgcgcagggcctgtgggcgcagatgtccgtgttccaccgagaggacctggaaaggctcggggtgcaggagtccgacctccgtctgttcctggacggagacatcctccgccaggacagagtctccaaaggctgctactccttcatccacctcagcttccagcagtttctcactgccctgttctacgccctggagaaggaggagggggaggacagggacggccacgcctgggacatcggggacgtacagaagctgctttccggagaagaaagactcaagaaccccgacctgattcaagtaggacacttcttattcggcctcgctaacgagaagagagccaaggagttggaggccacttttggctgccggatgtcaccggacatcaaacaggaattgctgcaatgcaaagcacatcttcatgcaaataagcccttatccgtgaccgacctgaaggaggtcttgggctgcctgtatgagtctcaggaggaggagctggcgaaggtggtggtggccccgttcaaggaaatttctattcacctgacaaatacttctgaagtgatgcattgttccttcagcctgaagcattgtcaagacttgcagaaactctcactgcaggtagcaaagggggtgttcctggagaattacatggattttgaactggacattgaatttgaaaggtgcacttacctaaccattccgaactgggctcggcaggatcttcgctctcttcgcctctggacagatttctgctctctcttcagctcaaacagcaacctcaagtttctggaagtgaaacaaagcttcctgagtgactcttctgtgcggattctttgtgaccacgtaacccgtagcacctgtcatctgcagaaagtggagattaaaaacgtcacccctgacaccgcgtaccgggacttctgtcttgctttcattgggaagaagaccctcacgcacctgaccctggcagggcacatcgagtgggaacgcacgatgatgctgatgctgtgtgacctgctcagaaatcataaatgcaacctgcagtacctgaggttgggaggtcactgtgccaccccggagcagtgggctgaattcttctatgtcctcaaagccaaccagtccctgaagcacctgcgtctctcagccaatgtgctcctggatgagggtgccatgttgctgtacaagaccatgacacgcccaaaacacttcctgcagatgttgtcgttggaaaactgtcgtcttacagaagccagttgcaaggaccttgctgctgtcttggttgtcagcaagaagctgacacacctgtgcttggccaagaaccccattggggatacaggggtgaagtttctgtgtgagggcttgagttaccctgattgtaaactgcagaccttggtgttacagcaatgcagcataaccaagcttggctgtagatatctctcagaggcgctccaagaagcctgcagcctcacaaacctggacttgagtatcaaccagatagctcgtggattgtggattctctgtcaggcattagagaatccaaactgtaacctaaaacacctacggttgaagacctatgaaactaatttggaaatcaagaagctgttggaggaagtgaaagaaaagaatcccaagctgactattgattgcaatgcttccggggcaacggcacctccgtgctgtgactttttttgctgagcagcctgggatcgctctacgaattacacaggaagcgggattcgggtctctaagatgtcttatgaatgcaggtcagagggtcacatgttaacactagagtctgtcgagaggtaggatttgacactggttttctcactatttttgggagattctgcacgagtcacgcacccccttcacatgacgctatgtactttctcacagggataataaagttagagcactctcgttgca

SEQ ID NO: 3 - protein, 980 aa, Homo sapiens
MTSPQLEWTLQTLLEQLNEDELKSFKSLLWAFPLEDVLQKTPWSEVEEADGKKLAEILVNTSSENWIRNATVNILEEMNLTELCKMAKAEMMEDGQVQEIDNPELGDAEEDSELAKPGEKEGWRNSMEKQSLVWKNTFWQGDIDNFHDDVTLRNQRFIPFLNPRTPRKLTPYTVVLHGPAGVGKTTLAKKCMLDWTDCNLSPTLRYAFYLSCKELSRMGPCSFAELISKDWPELQDDIPSILAQAQRILFVVDGLDELKVPPGALIQDICGDWEKKKPVPVLLGSLLKRKMLPRAALLVTTRPRALRDLQLLAQQPIYVRVEGFLEEDRRAYFLRHFGDEDQAMRAFELMRSNAALFQLGSAPAVCWIVCTTLKLQMEKGEDPVPTCLTRTGLFLRFLCSRFPQGAQLRGALRTLSLLAAQGLWAQMSVFHREDLERLGVQESDLRLFLDGDILRQDRVSKGCYSFIHLSFQQFLTALFYALEKEEGEDRDGHAWDIGDVQKLLSGEERLKNPDLIQVGHFLFGLANEKRAKELEATFGCRMSPDIKQELLQCKAHLHANKPLSVTDLKEVLGCLYESQEEELAKVVVAPFKEISIHLTNTSEVMHCSFSLKHCQDLQKLSLQVAKGVFLENYMDFELDIEFERCTYLTIPNWARQDLRSLRLWTDFCSLFSSNSNLKFLEVKQSFLSDSSVRILCDHVTRSTCHLQKVEIKNVTPDTAYRDFCLAFIGKKTLTHLTLAGHIEWERTMMLMLCDLLRNHKCNLQYLRLGGHCATPEQWAEFFYVLKANQSLKHLRLSANVLLDEGAMLLYKTMTRPKHFLQMLSLENCRLTEASCKDLAAVLVVSKKLTHLCLAKNPIGDTGVKFLCEGLSYPDCKLQTLVLQQCSITKLGCRYLSEALQEACSLTNLDLSINQIARGLWILCQALENPNCNLKHLRLKTYETNLEIKKLLEEVKEKNPKLTIDCNASGATAPPCCDFFC

SEQ ID NO: 4 - DNA, 3337 bp, Homo sapiens
  CDS (77)..(3106)
gaaacacaggctggaagcaagacctgacctgagggagttcttcagccttaacctaaggtctcatactcggagcactatgacatcgccccagctagagtggactctgcagacccttctggagcagctgaacgaggatgaattaaagagtttcaaatcccttttatgggcttttcccctcgaagacgtgctacagaagaccccatggtctgaggtggaagaggctgatggcaagaaactggcagaaattctggtcaacacctcctcagaaaattggataaggaatgcgactgtgaacatcttggaagagatgaatctcacggaattgtgtaagatggcaaaggctgagatgatggaggacggacaggtgcaagaaatagataatcctgagctgggagatgcagaagaagactcggagttagcaaagccaggtgaaaaggaaggatggagaaattcaatggagaaacagtctttggtctggaagaacaccttttggcaaggagacattgacaatttccatgacgacgtcactctgagaaaccaacggttcattccattcttgaatcccagaacacccaggaagctaacaccttacacggtggtgctgcacggccccgcaggcgtggggaaaaccacgctggccaaaaagtgtatgctggactggacagactgcaacctcagcccgacgctcagatacgcgttctacctcagctgcaaggagctcagccgcatgggcccctgcagttttgcagagctgatctccaaagactggcctgaattgcaggatgacattccaagcatcctagcccaagcacagagaatcctgttcgtggtcgatggccttgatgagctgaaagtcccacctggggcgctgatccaggacatctgcggggactgggagaagaagaagccggtgcccgtcctcctggggagtttgctgaagaggaagatgttacccagggcagccttgctggtcaccacgcggcccagggcactgagggacctccagctcctggcgcagcagccgatctacgtaagggtggagggcttcctggaggaggacaggagggcctatttcctgagacactttggagacgaggaccaagccatgcgtgcctttgagctaatgaggagcaacgcggccctgttccagctgggctcggcccccgcggtgtgctggattgtgtgcacgactctgaagctgcagatggagaagggggaggacccggtccccacctgcctcacccgcacggggctgttcctgcgtttcctctgcagccggttcccgcagggcgcacagctgcggggcgcgctgcggacgctgagcctcctggccgcgcagggcctgtgggcgcagatgtccgtgttccaccgagaggacctggaaaggctcggggtgcaggagtccgacctccgtctgttcctggacggagacatcctccgccaggacagagtctccaaaggctgctactccttcatccacctcagcttccagcagtttctcactgccctgttctacgccctggagaaggaggagggggaggacagggacggccacgcctgggacatcggggacgtacagaagctgctttccggagaagaaagactcaagaaccccgacctgattcaagtaggacacttcttattcggcctcgctaacgagaagagagccaaggagttggaggccacttttggctgccggatgtcaccggacatcaaacaggaattgctgcaatgcaaagcacatcttcatgcaaataagcccttatccgtgaccgacctgaaggaggtcttgggctgcctgtatgagtctcaggaggaggagctggcgaaggtggtggtggccccgttcaaggaaatttctattcacctgacaaatacttctgaagtgatgcattgttccttcagcctgaagcattgtcaagacttgcagaaactctcactgcaggtagcaaagggggtgttcctggagaattacatggattttgaactggacattgaatttgaaagctcaaacagcaacctcaagtttctggaagtgaaacaaagcttcctgagtgactcttctgtgcggattctttgtgaccacgtaacccgtagcacctgtcatctgcagaaagtggagattaaaaacgtcacccctgacaccgcgtaccgggacttctgtcttgctttcattgggaagaagaccctcacgcacctgaccctggcagggcacatcgagtgggaacgcacgatgatgctgatgctgtgtgacctgctcagaaatcataaatgcaacctgcagtacctgaggttgggaggtcactgtgccaccccggagcagtgggctgaattcttctatgtcctcaaagccaaccagtccctgaagcacctgcgtctctcagccaatgtgctcctggatgagggtgccatgttgctgtacaagaccatgacacgcccaaaacacttcctgcagatgttgtcgttggaaaactgtcgtcttacagaagccagttgcaaggaccttgctgctgtcttggttgtcagcaagaagctgacacacctgtgcttggccaagaaccccattggggatacaggggtgaagtttctgtgtgagggcttgagttaccctgattgtaaactgcagaccttggtgttacagcaatgcagcataaccaagcttggctgtagatatctctcagaggcgctccaagaagcctgcagcctcacaaacctggacttgagtatcaaccagatagctcgtggattgtggattctctgtcaggcattagagaatccaaactgtaacctaaaacacctacgcctctggagctgctccctcatgcctttctattgtcagcatcttggatctgctctcctcagcaatcagaagcttgaaactctggacctgggccagaatcatttgtggaagagtggcataattaagctctttggggttctaagacaaagaactggatccttgaagatactcaggttgaagacctatgaaactaatttggaaatcaagaagctgttggaggaagtgaaagaaaagaatcccaagctgactattgattgcaatgcttccggggcaacggcacctccgtgctgtgactttttttgctgagcagcctgggatcgctctacgaattacacaggaagcgggattcgggtctctaagatgtcttatgaatgcaggtcagagggtcacatgttaacactagagtctgtcgagaggtaggatttgacactggttttctcactatttttgggagattctgcacgagtcacgcacccccttcacatgacgctatgtactttctcacagggataataaagttagagcactctcgttgca

SEQ ID NO: 5 - protein, 1009 aa, Homo sapiens
MTSPQLEWTLQTLLEQLNEDELKSFKSLLWAFPLEDVLQKTPWSEVEEADGKKLAEILVNTSSENWIRNATVNILEEMNLTELCKMAKAEMMEDGQVQEIDNPELGDAEEDSELAKPGEKEGWRNSMEKQSLVWKNTFWQGDIDNFHDDVTLRNQRFIPFLNPRTPRKLTPYTVVLHGPAGVGKTTLAKKCMLDWTDCNLSPTLRYAFYLSCKELSRMGPCSFAELISKDWPELQDDIPSILAQAQRILFVVDGLDELKVPPGALIQDICGDWEKKKPVPVLLGSLLKRKMLPRAALLVTTRPRALRDLQLLAQQPIYVRVEGFLEEDRRAYFLRHFGDEDQAMRAFELMRSNAALFQLGSAPAVCWIVCTTLKLQMEKGEDPVPTCLTRTGLFLRFLCSRFPQGAQLRGALRTLSLLAAQGLWAQMSVFHREDLERLGVQESDLRLFLDGDILRQDRVSKGCYSFIHLSFQQFLTALFYALEKEEGEDRDGHAWDIGDVQKLLSGEERLKNPDLIQVGHFLFGLANEKRAKELEATFGCRMSPDIKQELLQCKAHLHANKPLSVTDLKEVLGCLYESQEEELAKVVVAPFKEISIHLTNTSEVMHCSFSLKHCQDLQKLSLQVAKGVFLENYMDFELDIEFESSNSNLKFLEVKQSFLSDSSVRILCDHVTRSTCHLQKVEIKNVTPDTAYRDFCLAFIGKKTLTHLTLAGHIEWERTMMLMLCDLLRNHKCNLQYLRLGGHCATPEQWAEFFYVLKANQSLKHLRLSANVLLDEGAMLLYKTMTRPKHFLQMLSLENCRLTEASCKDLAAVLVVSKKLTHLCLAKNPIGDTGVKFLCEGLSYPDCKLQTLVLQQCSITKLGCRYLSEALQEACSLTNLDLSINQIARGLWILCQALENPNCNLKHLRLWSCSLMPFYCQHLGSALLSNQKLETLDLGQNHLWKSGIIKLFGVLRQRTGSLKILRLKTYETNLEIKKLLEEVKEKNPKLTIDCNASGATAPPCCDFFC

SEQ ID NO: 6 - DNA, 3421 bp, Homo sapiens
  CDS (77)..(3190)
gaaacacaggctggaagcaagacctgacctgagggagttcttcagccttaacctaaggtctcatactcggagcactatgacatcgccccagctagagtggactctgcagacccttctggagcagctgaacgaggatgaattaaagagtttcaaatcccttttatgggcttttcccctcgaagacgtgctacagaagaccccatggtctgaggtggaagaggctgatggcaagaaactggcagaaattctggtcaacacctcctcagaaaattggataaggaatgcgactgtgaacatcttggaagagatgaatctcacggaattgtgtaagatggcaaaggctgagatgatggaggacggacaggtgcaagaaatagataatcctgagctgggagatgcagaagaagactcggagttagcaaagccaggtgaaaaggaaggatggagaaattcaatggagaaacagtctttggtctggaagaacaccttttggcaaggagacattgacaatttccatgacgacgtcactctgagaaaccaacggttcattccattcttgaatcccagaacacccaggaagctaacaccttacacggtggtgctgcacggccccgcaggcgtggggaaaaccacgctggccaaaaagtgtatgctggactggacagactgcaacctcagcccgacgctcagatacgcgttctacctcagctgcaaggagctcagccgcatgggcccctgcagttttgcagagctgatctccaaagactggcctgaattgcaggatgacattccaagcatcctagcccaagcacagagaatcctgttcgtggtcgatggccttgatgagctgaaagtcccacctggggcgctgatccaggacatctgcggggactgggagaagaagaagccggtgcccgtcctcctggggagtttgctgaagaggaagatgttacccagggcagccttgctggtcaccacgcggcccagggcactgagggacctccagctcctggcgcagcagccgatctacgtaagggtggagggcttcctggaggaggacaggagggcctatttcctgagacactttggagacgaggaccaagccatgcgtgcctttgagctaatgaggagcaacgcggccctgttccagctgggctcggcccccgcggtgtgctggattgtgtgcacgactctgaagctgcagatggagaagggggaggacccggtccccacctgcctcacccgcacggggctgttcctgcgtttcctctgcagccggttcccgcagggcgcacagctgcggggcgcgctgcggacgctgagcctcctggccgcgcagggcctgtgggcgcagatgtccgtgttccaccgagaggacctggaaaggctcggggtgcaggagtccgacctccgtctgttcctggacggagacatcctccgccaggacagagtctccaaaggctgctactccttcatccacctcagcttccagcagtttctcactgccctgttctacgccctggagaaggaggagggggaggacagggacggccacgcctgggacatcggggacgtacagaagctgctttccggagaagaaagactcaagaaccccgacctgattcaagtaggacacttcttattcggcctcgctaacgagaagagagccaaggagttggaggccacttttggctgccggatgtcaccggacatcaaacaggaattgctgcaatgcaaagcacatcttcatgcaaataagcccttatccgtgaccgacctgaaggaggtcttgggctgcctgtatgagtctcaggaggaggagctggcgaaggtggtggtggccccgttcaaggaaatttctattcacctgacaaatacttctgaagtgatgcattgttccttcagcctgaagcattgtcaagacttgcagaaactctcactgcaggtagcaaagggggtgttcctggagaattacatggattttgaactggacattgaatttgaaaggtgcacttacctaaccattccgaactgggctcggcaggatcttcgctctcttcgcctctggacagatttctgctctctcttcagctcaaacagcaacctcaagtttctggaagtgaaacaaagcttcctgagtgactcttctgtgcggattctttgtgaccacgtaacccgtagcacctgtcatctgcagaaagtggagattaaaaacgtcacccctgacaccgcgtaccgggacttctgtcttgctttcattgggaagaagaccctcacgcacctgaccctggcagggcacatcgagtgggaacgcacgatgatgctgatgctgtgtgacctgctcagaaatcataaatgcaacctgcagtacctgaggttgggaggtcactgtgccaccccggagcagtgggctgaattcttctatgtcctcaaagccaaccagtccctgaagcacctgcgtctctcagccaatgtgctcctggatgagggtgccatgttgctgtacaagaccatgacacgcccaaaacacttcctgcagatgttgtcgttggaaaactgtcgtcttacagaagccagttgcaaggaccttgctgctgtcttggttgtcagcaagaagctgacacacctgtgcttggccaagaaccccattggggatacaggggtgaagtttctgtgtgagggcttgagttaccctgattgtaaactgcagaccttggtgttacagcaatgcagcataaccaagcttggctgtagatatctctcagaggcgctccaagaagcctgcagcctcacaaacctggacttgagtatcaaccagatagctcgtggattgtggattctctgtcaggcattagagaatccaaactgtaacctaaaacacctacgcctctggagctgctccctcatgcctttctattgtcagcatcttggatctgctctcctcagcaatcagaagcttgaaactctggacctgggccagaatcatttgtggaagagtggcataattaagctctttggggttctaagacaaagaactggatccttgaagatactcaggttgaagacctatgaaactaatttggaaatcaagaagctgttggaggaagtgaaagaaaagaatcccaagctgactattgattgcaatgcttccggggcaacggcacctccgtgctgtgactttttttgctgagcagcctgggatcgctctacgaattacacaggaagcgggattcgggtctctaagatgtcttatgaatgcaggtcagagggtcacatgttaacactagagtctgtcgagaggtaggatttgacactggttttctcactatttttgggagattctgcacgagtcacgcacccccttcacatgacgctatgtactttctcacagggataataaagttagagcactctcgttgca

SEQ ID NO: 7 - protein, 1037 aa, Homo sapiens
MTSPQLEWTLQTLLEQLNEDELKSFKSLLWAFPLEDVLQKTPWSEVEEADGKKLAEILVNTSSENWIRNATVNILEEMNLTELCKMAKAEMMEDGQVQEIDNPELGDAEEDSELAKPGEKEGWRNSMEKQSLVWKNTFWQGDIDNFHDDVTLRNQRFIPFLNPRTPRKLTPYTVVLHGPAGVGKTTLAKKCMLDWTDCNLSPTLRYAFYLSCKELSRMGPCSFAELISKDWPELQDDIPSILAQAQRILFVVDGLDELKVPPGALIQDICGDWEKKKPVPVLLGSLLKRKMLPRAALLVTTRPRALRDLQLLAQQPIYVRVEGFLEEDRRAYFLRHFGDEDQAMRAFELMRSNAALFQLGSAPAVCWIVCTTLKLQMEKGEDPVPTCLTRTGLFLRFLCSRFPQGAQLRGALRTLSLLAAQGLWAQMSVFHREDLERLGVQESDLRLFLDGDILRQDRVSKGCYSFIHLSFQQFLTALFYALEKEEGEDRDGHAWDIGDVQKLLSGEERLKNPDLIQVGHFLFGLANEKRAKELEATFGCRMSPDIKQELLQCKAHLHANKPLSVTDLKEVLGCLYESQEEELAKVVVAPFKEISIHLTNTSEVMHCSFSLKHCQDLQKLSLQVAKGVFLENYMDFELDIEFERCTYLTIPNWARQDLRSLRLWTDFCSLFSSNSNLKFLEVKQSFLSDSSVRILCDHVTRSTCHLQKVEIKNVTPDTAYRDFCLAFIGKKTLTHLTLAGHIEWERTMMLMLCDLLRNHKCNLQYLRLGGHCATPEQWAEFFYVLKANQSLKHLRLSANVLLDEGAMLLYKTMTRPKHFLQMLSLENCRLTEASCKDLAAVLVVSKKLTHLCLAKNPIGDTGVKFLCEGLSYPDCKLQTLVLQQCSITKLGCRYLSEALQEACSLTNLDLSINQIARGLWILCQALENPNCNLKHLRLWSCSLMPFYCQHLGSALLSNQKLETLDLGQNHLWKSGIIKLFGVLRQRTGSLKILRLKTYETNLEIKKLLEEVKEKNPKLTIDCNASGATAPPCCDFFC

SEQ ID NO: 8 - DNA, 3114 bp, Homo sapiens
atgacatcgccccagctagagtggactctgcagacccttctggagcagctgaacgaggatgaattaaagagtttcaaatcccttttatgggcttttcccctcgaagacgtgctacagaagaccccatggtctgaggtggaagaggctgatggcaagaaactggcagaaattctggtcaacacctcctcagaaaattggataaggaatgcgactgtgaacatcttggaagagatgaatctcacggaattgtgtaagatggcaaaggctgagatgatggaggacggacaggtgcaagaaatagataatcctgagctgggagatgcagaagaagactcggagttagcaaagccaggtgaaaaggaaggatggagaaattcaatggagaaacagtctttggtctggaagaacaccttttggcaaggagacattgacaatttccatgacgacgtcactctgagaaaccaacggttcattccattcttgaatcccagaacacccaggaagctaacaccttacacggtggtgctgcacggccccgcaggcgtggggaaaaccacgctggccaaaaagtgtatgctggactggacagactgcaacctcagcccgacgctcagatacgcgttctacctcagctgcaaggagctcagccgcatgggcccctgcagttttgcagagctgatctccaaagactggcctgaattgcaggatgacattccaagcatcctagcccaagcacagagaatcctgttcgtggtcgatggccttgatgagctgaaagtcccacctggggcgctgatccaggacatctgcggggactgggagaagaagaagccggtgcccgtcctcctggggagtttgctgaagaggaagatgttacccagggcagccttgctggtcaccacgcggcccagggcactgagggacctccagctcctggcgcagcagccgatctacgtaagggtggagggcttcctggaggaggacaggagggcctatttcctgagacactttggagacgaggaccaagccatgcgtgcctttgagctaatgaggagcaacgcggccctgttccagctgggctcggcccccgcggtgtgctggattgtgtgcacgactctgaagctgcagatggagaagggggaggacccggtccccacctgcctcacccgcacggggctgttcctgcgtttcctctgcagccggttcccgcagggcgcacagctgcggggcgcgctgcggacgctgagcctcctggccgcgcagggcctgtgggcgcagatgtccgtgttccaccgagaggacctggaaaggctcggggtgcaggagtccgacctccgtctgttcctggacggagacatcctccgccaggacagagtctccaaaggctgctactccttcatccacctcagcttccagcagtttctcactgccctgttctacgccctggagaaggaggagggggaggacagggacggccacgcctgggacatcggggacgtacagaagctgctttccggagaagaaagactcaagaaccccgacctgattcaagtaggacacttcttattcggcctcgctaacgagaagagagccaaggagttggaggccacttttggctgccggatgtcaccggacatcaaacaggaattgctgcaatgcaaagcacatcttcatgcaaataagcccttatccgtgaccgacctgaaggaggtcttgggctgcctgtatgagtctcaggaggaggagctggcgaaggtggtggtggccccgttcaaggaaatttctattcacctgacaaatacttctgaagtgatgcattgttccttcagcctgaagcattgtcaagacttgcagaaactctcactgcaggtagcaaagggggtgttcctggagaattacatggattttgaactggacattgaatttgaaaggtgcacttacctaaccattccgaactgggctcggcaggatcttcgctctcttcgcctctggacagatttctgctctctcttcagctcaaacagcaacctcaagtttctggaagtgaaacaaagcttcctgagtgactcttctgtgcggattctttgtgaccacgtaacccgtagcacctgtcatctgcagaaagtggagattaaaaacgtcacccctgacaccgcgtaccgggacttctgtcttgctttcattgggaagaagaccctcacgcacctgaccctggcagggcacatcgagtgggaacgcacgatgatgctgatgctgtgtgacctgctcagaaatcataaatgcaacctgcagtacctgaggttgggaggtcactgtgccaccccggagcagtgggctgaattcttctatgtcctcaaagccaaccagtccctgaagcacctgcgtctctcagccaatgtgctcctggatgagggtgccatgttgctgtacaagaccatgacacgcccaaaacacttcctgcagatgttgtcgttggaaaactgtcgtcttacagaagccagttgcaaggaccttgctgctgtcttggttgtcagcaagaagctgacacacctgtgcttggccaagaaccccattggggatacaggggtgaagtttctgtgtgagggcttgagttaccctgattgtaaactgcagaccttggtgttacagcaatgcagcataaccaagcttggctgtagatatctctcagaggcgctccaagaagcctgcagcctcacaaacctggacttgagtatcaaccagatagctcgtggattgtggattctctgtcaggcattagagaatccaaactgtaacctaaaacacctacgcctctggagctgctccctcatgcctttctattgtcagcatcttggatctgctctcctcagcaatcagaagcttgaaactctggacctgggccagaatcatttgtggaagagtggcataattaagctctttggggttctaagacaaagaactggatccttgaagatactcaggttgaagacctatgaaactaatttggaaatcaagaagctgttggaggaagtgaaagaaaagaatcccaagctgactattgattgcaatgcttccggggcaacggcacctccgtgctgtgactttttttgctga

SEQ ID NO: 9 - DNA, 3421 bp, Homo sapiens
gaaacacaggctggaagcaagacctgacctgagggagttcttcagccttaacctaaggtctcatactcggagcactatgacatcgccccagctagagtggactctgcagacccttctggagcagctgaacgaggatgaattaaagagtttcaaatcccttttatgggcttttcccctcgaagacgtgctacagaagaccccatggtctgaggtggaagaggctgatggcaagaaactggcagaaattctggtcaacacctcctcagaaaattggataaggaatgcgactgtgaacatcttggaagagatgaatctcacggaattgtgtaagatggcaaaggctgagatgatggaggacggacaggtgcaagaaatagataatcctgagctgggagatgcagaagaagactcggagttagcaaagccaggtgaaaaggaaggatggagaaattcaatggagaaacagtctttggtctggaagaacaccttttggcaaggagacattgacaatttccatgacgacgtcactctgagaaaccaacggttcattccattcttgaatcccagaacacccaggaagctaacaccttacacggtggtgctgcacggccccgcaggcgtggggaaaaccacgctggccaaaaagtgtatgctggactggacagactgcaacctcagcccgacgctcagatacgcgttctacctcagctgcaaggagctcagccgcatgggcccctgcagttttgcagagctgatctccaaagactggcctgaattgcaggatgacattccaagcatcctagcccaagcacagagaatcctgttcgtggtcgatggccttgatgagctgaaagtcccacctggggcgctgatccaggacatctgcggggactgggagaagaagaagccggtgcccgtcctcctggggagtttgctgaagaggaagatgttacccagggcagccttgctggtcaccacgcggcccagggcactgagggacctccagctcctggcgcagcagccgatctacgtaagggtggagggcttcctggaggaggacaggagggcctatttcctgagacactttggagacgaggaccaagccatgcgtgcctttgagctaatgaggagcaacgcggccctgttccagctgggctcggcccccgcggtgtgctggattgtgtgcacgactctgaagctgcagatggagaagggggaggacccggtccccacctgcctcacccgcacggggctgttcctgcgtttcctctgcagccggttcccgcagggcgcacagctgcggggcgcgctgcggacgctgagcctcctggccgcgcagggcctgtgggcgcagatgtccgtgttccaccgagaggacctggaaaggctcggggtgcaggagtccgacctccgtctgttcctggacggagacatcctccgccaggacagagtctccaaaggctgctactccttcatccacctcagcttccagcagtttctcactgccctgttctacgccctggagaaggaggagggggaggacagggacggccacgcctgggacatcggggacgtacagaagctgctttccggagaagaaagactcaagaaccccgacctgattcaagtaggacacttcttattcggcctcgctaacgagaagagagccaaggagttggaggccacttttggctgccggatgtcaccggacatcaaacaggaattgctgcaatgcaaagcacatcttcatgcaaataagcccttatccgtgaccgacctgaaggaggtcttgggctgcctgtatgagtctcaggaggaggagctggcgaaggtggtggtggccccgttcaaggaaatttctattcacctgacaaatacttctgaagtgatgcattgttccttcagcctgaagcattgtcaagacttgcagaaactctcactgcaggtagcaaagggggtgttcctggagaattacatggattttgaactggacattgaatttgaaaggtgcacttacctaaccattccgaactgggctcggcaggatcttcgctctcttcgcctctggacagatttctgctctctcttcagctcaaacagcaacctcaagtttctggaagtgaaacaaagcttcctgagtgactcttctgtgcggattctttgtgaccacgtaacccgtagcacctgtcatctgcagaaagtggagattaaaaacgtcacccctgacaccgcgtaccgggacttctgtcttgctttcattgggaagaagaccctcacgcacctgaccctggcagggcacatcgagtgggaacgcacgatgatgctgatgctgtgtgacctgctcagaaatcataaatgcaacctgcagtacctgaggttgggaggtcactgtgccaccccggagcagtgggctgaattcttctatgtcctcaaagccaaccagtccctgaagcacctgcgtctctcagccaatgtgctcctggatgagggtgccatgttgctgtacaagaccatgacacgcccaaaacacttcctgcagatgttgtcgttggaaaactgtcgtcttacagaagccagttgcaaggaccttgctgctgtctggaaaactgtcgtcttacagaagccagttgcaaggaccttgctgctgtctggggatacaggggtgaagtttctgtgtgagggcttgagttaccctgattgtaaactgcagaccttggtgttacagcaatgcagcataaccaagcttggctgtagatatctctcagaggcgctccaagaagcctgcagcctcacaaacctggacttgagtatcaaccagatagctcgtggattgtggattctctgtcaggcattagagaatccaaactgtaacctaaaacacctacgcctctggagctgctccctcatgcctttctattgtcagcatcttggatctgctctcctcagcaatcagaagcttgaaactctggacctgggccagaatcatttgtggaagagtggcataattaagctctttggggttctaagacaaagaactggatccttgaagatactcaggttgaagacctatgaaactaatttggaaatcaagaagctgttggaggaagtgaaagaaaagaatcccaagctgactattgattgcaatgcttccggggcaacggcacctccgtgctgtgactttttttgctgagcagcctgggatcgctctacgaattacacaggaagcgggattcgggtctctaagatgtcttatgaatgcaggtcagagggtcacatgttaacactagagtctgtcgagaggtaggatttgacactggttttctcactatttttgggagattctgcacgagtcacgcacccccttcacatgacgctatgtactttctcacagggataataaagttagagcactctcgttgca